SEQUENCE LISTING (172 sequences)

SEQ ID NO: 1 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 2 - protein, 24 aa, Komagataella pastoris
MIFDGTTMSIAIGLLSTLGIGAEA

SEQ ID NO: 3 - protein, 524 aa, Cannabis sativa
FNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 4 - protein, 523 aa, Cannabis sativa
NIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 5 - protein, 522 aa, Cannabis sativa
IQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 6 - protein, 521 aa, Cannabis sativa
QTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 7 - protein, 519 aa, Cannabis sativa
SIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 8 - protein, 518 aa, Cannabis sativa
IANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 9 - protein, 517 aa, Cannabis sativa
ANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 10 - protein, 516 aa, Cannabis sativa
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 11 - protein, 515 aa, Cannabis sativa
PRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 12 - protein, 514 aa, Cannabis sativa
RENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 13 - protein, 513 aa, Cannabis sativa
ENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 14 - protein, 512 aa, Cannabis sativa
NFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 15 - protein, 516 aa, Cannabis sativa
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPQPRHRH

SEQ ID NO: 16 - protein, 516 aa, Cannabis sativa
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNSRLAYLNYRDLDIGINDPKNPNNYTQARIWGEKYFGKNFDRLVKVKTLVDPNNFFRNEQSIPPLPRHRH

SEQ ID NO: 17 - protein, 516 aa, Cannabis sativa
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVRSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 18 - protein, 516 aa, Cannabis sativa
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNSRLAYLNYRDLDIGINDPKNPNNYTQARIWGEKYFGKNFDRLVKVKTLVDPNNFFRNEQSIPPQPRHRH

SEQ ID NO: 19 - protein, 516 aa, Cannabis sativa
NPRENFLKCFSQYIPNNATNLKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYWVNEKNESLSLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHRH

SEQ ID NO: 20 - protein, 516 aa, Cannabis sativa
NPRENFLKCFSQYIPNNATNLKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYWVNEKNESLSLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIRINDPKNPNNYTQARIWGEKYFGKNFDRLVKVKTLVDPNNFFRNEQSIPPLPRHRH

SEQ ID NO: 21 - protein, 516 aa, Cannabis sativa
NPRENFLKCFSQYIPNNATNLKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYWVNEKNESLSLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSARQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHRH

SEQ ID NO: 22 - protein, 516 aa, Cannabis sativa
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPHVSQNSRLAYINYRDLDIGINDPKNPNNYTQARIWGEKYFGKNFDRLVKVKTLVDPNNFFRNEQSIPPLPRHRH

SEQ ID NO: 23 - protein, 516 aa, Cannabis sativa
NPRENFLKCFSQYIPNNATNLKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYWVNEKNESLSLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKHPNNPTHARIRAQKYFRQNFDKLVKVKTLVDPNNFFRNEQSIPPLPRHRH

SEQ ID NO: 24 - protein, 1009 aa, Saccharomyces cerevisiae
MTMPHRYMFLAVFTLLALTSVASGATEACLPAGQRKSGMNINFYQYSLKDSSTYSNAAYMAYGYASKTKLGSVGGQTDISIDYNIPCVSSSGTFPCPQEDSYGNWGCKGMGACSNSQGIAYWSTDLFGFYTTPTNVTLEMTGYFLPPQTGSYTFKFATVDDSAILSVGGATAFNCCAQQQPPITSTNFTIDGIKPWGGSLPPNIEGTVYMYAGYYYPMKVVYSNAVSWGTLPISVTLPDGTTVSDDFEGYVYSFDDDLSQSNCTVPDPSNYAVSTTTTTTEPWTGTFTSTSTEMTTVTGTNGVPTDETVIVIRTPTTASTIITTTEPWNSTFTSTSTELTTVTGTNGVRTDETIIVIRTPTTATTAITTTEPWNSTFTSTSTELTTVTGTNGLPTDETIIVIRTPTTATTAMTTTQPWNDTFTSTSTELTTVTGTNGLPTDETIIVIRTPTTATTAMTTTQPWNDTFTSTSTELTTVTGTNGLPTDETIIVIRTPTTATTAMTTTQPWNDTFTSTSTEITTVTGTNGLPTDETIIVIRTPTTATTAMTTPQPWNDTFTSTSTEMTTVTGTNGLPTDETIIVIRTPTTATTAITTTEPWNSTFTSTSTEMTTVTGTNGLPTDETIIVIRTPTTATTAITTTQPWNDTFTSTSTEMTTVTGTNGLPTDETIIVIRTPTTATTAMTTTQPWNDTFTSTSTEITTVTGTTGLPTDETIIVIRTPTTATTAMTTTQPWNDTFTSTSTEMTTVTGTNGVPTDETVIVIRTPTSEGLISTTTEPWTGTFTSTSTEMTTVTGTNGQPTDETVIVIRTPTSEGLVTTTTEPWTGTFTSTSTEMTTITGTNGVPTDETVIVIRTPTSEGLISTTTEPWTGTFTSTSTEMTTITGTNGQPTDETVIVIRTPTSEGLISTTTEPWTGTFTSTSTEMTHVTGTNGVPTDETVIVIRTPTSEGLISTTTEPWTGTFTSTSTEVTTITGTNGQPTDETVIVIRTPTSEGLISTTTEPWTGTFTS

SEQ ID NO: 25 - protein, 341 aa, Saccharomyces cerevisiae
MQYKKSLVASALVATSLAAYAPKDPWSTLTPSATYKGGITDYSSTFGIAVEPIATTASSKAKRAAAISQIGDGQIQATTKTTAAAVSQIGDGQIQATTKTKAAAVSQIGDGQIQATTKTTSAKTTAAAVSQIGDGQIQATTKTKAAAVSQIGDGQIQATTKTTAAAVSQIGDGQIQATTKTTAAAVSQIGDGQIQATTNTTVAPVSQITDGQIQATTLTSATIIPSPAPAPITNGTDPVTAETCKSSGTLEMNLKGGILTDGKGRIGSIVANRQFQFDGPPPQAGAIYAAGWSITPEGNLAIGDQDTFYQCLSGNFYNLYDEHIGTQCNAVHLQAIDLLNC

SEQ ID NO: 26 - protein, 413 aa, Saccharomyces cerevisiae
MQYKKTLVASALAATTLAAYAPSEPWSTLTPTATYSGGVTDYASTFGIAVQPISTTSSASSAATTASSKAKRAASQIGDGQVQAATTTASVSTKSTAAAVSQIGDGQIQATTKTTAAAVSQIGDGQIQATTKTTSAKTTAAAVSQISDGQIQATTTTLAPKSTAAAVSQIGDGQVQATTTTLAPKSTAAAVSQIGDGQVQATTKTTAAAVSQIGDGQVQATTKTTAAAVSQIGDGQVQATTKTTAAAVSQIGDGQVQATTKTTAAAVSQITDGQVQATTKTTQAASQVSDGQVQATTATSASAAATSTDPVDAVSCKTSGTLEMNLKGGILTDGKGRIGSIVANRQFQFDGPPPQAGAIYAAGWSITPDGNLAIGDNDVFYQCLSGTFYNLYDEHIGSQCTPVHLEAIDLIDC

SEQ ID NO: 27 - protein, 325 aa, Saccharomyces cerevisiae
MQYKKPLVVSALAATSLAAYAPKDPWSTLTPSATYKGGITDYSSSFGIAIEAVATSASSVASSKAKRAASQIGDGQVQAATTTAAVSKKSTAAAVSQITDGQVQAAKSTAAAVSQITDGQVQAAKSTAAAVSQITDGQVQAAKSTAAAVSQITDGQVQAAKSTAAAASQISDGQVQATTSTKAAASQITDGQIQASKTTSGASQVSDGQVQATAEVKDANDPVDVVSCNNNSTLSMSLSKGILTDRKGRIGSIVANRQFQFDGPPPQAGAIYAAGWSITPEGNLALGDQDTFYQCLSGDFYNLYDKHIGSQCHEVYLQAIDLIDC

SEQ ID NO: 28 - protein, 227 aa, Saccharomyces cerevisiae
MQFKNVALAASVAALSATASAEGYTPGEPWSTLTPTGSISCGAAEYTTTFGIAVQAITSSKAKRDVISQIGDGQVQATSAATAQATDSQAQATTTATPTSSEKISSSASKTSTNATSSSCATPSLKDSSCKNSGTLELTLKDGVLTDAKGRIGSIVANRQFQFDGPPPQAGAIYAAGWSITEDGYLALGDSDVFYQCLSGNFYNLYDQNVAEQCSAIHLEAVSLVDC

SEQ ID NO: 29 - protein, 575 aa, Saccharomyces cerevisiae
TVVSSSAIEPSSASIISPVTSTLSSTTSSNPTTTSLSSTSTSPSSTSTSPSSTSTSSSSTSTSSSSTSTSSSSTSTSPSSTSTSSSLTSTSSSSTSTSQSSTSTSSSSTSTSPSSTSTSSSSTSTSPSSKSTSASSTSTSSYSTSTSPSLTSSSPTLASTSPSSTSISSTFTDSTSSLGSSIASSSTSVSLYSPSTPVYSVPSTSSNVATPSMTSSTVETTVSSQSSSEYITKSSISTTIPSFSMSTYFTTVSGVTTMYTTWCPYSSESETSTLTSMHETVTTDATVCTHESCMPSQTTSLITSSIKMSTKNVATSVSTSTVESSYACSTCAETSHSYSSVQTASSSSVTQQTTSTKSWVSSMTTSDEDFNKHATGKYHVTSSGTSTISTSVSEATSTSSIDSESQEQSSHLLSTSVLSSSSLSATLSSDSTILLFSSVSSLSVEQSPVTTLQISSTSEILQPTSSTAIATISASTSSLSATSISTPSTSVESTIESSSLTPTVSSIFLSSSSAPSSLQTSVTTTEVSTTSISIQYQTSSMVTISQYMGSGSQTRLPLGKLVFAIMAVACNVIFS

SEQ ID NO: 30 - protein, 73 aa, Saccharomyces cerevisiae
VDDVITQYTTWCPLTTEAPKNGTSTAAPVTSTEAPKNTTSAAPTHSVTSYTGAAAKALPAAGALLAGAAALLL

SEQ ID NO: 31 - protein, 213 aa, Saccharomyces cerevisiae
LVSIRSGSDLQYLSVYSDNGTLKLGSGSGSFEATITDDGKLKFDDDKYAVVNEDGSFKEGSESDAATGFSIKDGHLNYKSSSGFYAIKDGSSYIFSSKQSDDATGVAIRPTSKSGSVAADFSPSDSSSSSSASASSASASSSTKHSSSIESVETSTTVETSSASSPTASVISQITDGQIQAPNTVYEQTENAGAKAAVGMGAGALAVAAAYLL

SEQ ID NO: 32 - protein, 67 aa, Saccharomyces cerevisiae
ISQITDGQIQATTTATTEATTTAAPSSTVETVSPSSTETISQQTENGAAKAAVGMGAGALAAAAMLL

SEQ ID NO: 33 - protein, 401 aa, Saccharomyces cerevisiae
SVASFASSSPLLVSSRSNCSDARSSNTISSGLFSTIENVRNATSTFTNLSTDEIVITSCKSSCTNEDSVLTKTQVSTVETTITSCSGGICTTLMSPVTTINAKANTLTTTETSTVETTITTCPGGVCSTLTVPVTTITSEATTTATISCEDNEEDITSTETELLTLETTITSCSGGICTTLMSPVTTINAKANTLTTTETSTVETTITTCSGGVCSTLTVPVTTITSEATTTATISCEDNEEDVASTKTELLTMETTITSCSGGICTTLMSPVSSFNSKATTSNNAESTIPKAIKVSCSAGACTTLTTVDAGISMFTRTGLSITQTTVTNCSGGTCTMLTAPIATATSKVISPIPKASSATSIAHSSASYTVSINTNGAYNFDKDNIFGTAIVAVVALLLL

SEQ ID NO: 34 - protein, 417 aa, Saccharomyces cerevisiae
FYPSNGTSVISSSVISSSVTSSLVTSSSFISSSVISSSTTTSTSIFSESSTSSVIPTSSSTSGSSESKTSSASSSSSSSSISSESPKSPTNSSSSLPPVTSATTGQETASSLPPATTTKTSEQTTLVTVTSCESHVCTESISSAIVSTATVTVSGVTTEYTTWCPISTTETTKQTKGTTEQTKGTTEQTTETTKQTTVVTISSCESDICSKTASPAIVSTSTATINGVTTEYTTWCPISTTESKQQTTLVTVTSCESGVCSETTSPAIVSTATATVNDVVTVYPTWRPQTTNEQSVSSKMNSATSETTTNTGAAETKTAVTSSLSRFNHAETQTASATDVIGHNNSVVSVSETGNTKSLTSSGLSTMSQQPRSTPASSMVGYSTASLEISTYAGSANSLLAGSGLSVFIASLLLAII

SEQ ID NO: 35 - protein, 81 aa, Saccharomyces cerevisiae
SSTSLGARTTTGSNGRSTTSQQDGSAMHQPTSSIYTQLKEGTSTTAKLSAYEGAATPLSIFQCNSLAGTIAAFVVAVLFAF

SEQ ID NO: 36 - protein, 320 aa, Saccharomyces cerevisiae
SAKSSFISTTTTDLTSINTSAYSTGSISTVETGNRTTSEVISHVVTTSTKLSPTATTSLTIAQTSIYSTDSNITVGTDIHTTSEVISDVETISRETASTVVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 37 - protein, 229 aa, Saccharomyces cerevisiae
ALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 38 - protein, 96 aa, Saccharomyces cerevisiae
SSEASSSAATSSAVASSSEATSSTVASSTKAASSTKASSSAVSSAVASSTKASAISQISDGQVQATSTVSEQTENGAAKAVIGMGAGVMAAAAMLL

SEQ ID NO: 39 - protein, 144 aa, Saccharomyces cerevisiae
MTYTDDAYTTLFSELDFDAITKTIVKLPWYTTRLSSEIAAALASVSPASSEAASSSEAASSSKAASSSEATSSAAPSSSAAPSSSAAPSSSAESSSKAVSSSVAPTTSSVSTSTVETASNAGQRVNAGAASFGAVVAGAAALLL

SEQ ID NO: 40 - protein, 212 aa, Saccharomyces cerevisiae
SLASDSSSGFSLSSMPAGVLDIGMALASATDDSYTTLYSEVDFAGVSKMLTMVPWYSSRLEPALKSLNGDASSSAAPSSSAAPTSSAAPSSSAAPTSSAASSSSEAKSSSAAPSSSEAKSSSAAPSSSEAKSSSAAPSSSEAKSSSAAPSSTEAKITSAAPSSTGAKTSAISQITDGQIQATKAVSEQTENGAAKAFVGMGAGVVAAAAMLL

SEQ ID NO: 41 - protein, 65 aa, Saccharomyces cerevisiae
TSMVSTVKTTSTPYTTSTIATLSTKSISSQANTTTHEISTYVGAAVKGSVAGMGAIMGAAAFALL

SEQ ID NO: 42 - protein, 18 aa, Saccharomyces cerevisiae
MQLLRCFSIFSVIASVLA

SEQ ID NO: 43 - protein, 24 aa, Saccharomyces cerevisiae
MTLSFAHFTYLFTILLGLTNIALA

SEQ ID NO: 44 - protein, 20 aa, Saccharomyces cerevisiae
MQFSTVASVAFVALANFVAA

SEQ ID NO: 45 - protein, 18 aa, Saccharomyces cerevisiae
MQFSTVASIAAVAAVASA

SEQ ID NO: 46 - protein, 18 aa, Saccharomyces cerevisiae
MQYKKSLVASALVATSLA

SEQ ID NO: 47 - protein, 18 aa, Saccharomyces cerevisiae
MQYKKPLVVSALAATSLA

SEQ ID NO: 48 - protein, 18 aa, Saccharomyces cerevisiae
MAYIKIALLAAIAALASA

SEQ ID NO: 49 - protein, 36 aa, Saccharomyces cerevisiae
MESVSSLFNIFSTIMVNYKSLVLALLSVSNLKYARG

SEQ ID NO: 50 - protein, 24 aa, Saccharomyces cerevisiae
MSAINHLCLKLILASFAIINTITA

SEQ ID NO: 51 - protein, 19 aa, Saccharomyces cerevisiae
MVNISIVAGIVALATSAAA

SEQ ID NO: 52 - protein, 22 aa, Saccharomyces cerevisiae
MRQVWFSWIVGLFLCFFNVSSA

SEQ ID NO: 53 - protein, 19 aa, Saccharomyces cerevisiae
MLLQAFLFLLAGFAAKISA

SEQ ID NO: 54 - protein, 22 aa, Saccharomyces cerevisiae
MFSLKALLPLALLLVSANQVAA

SEQ ID NO: 55 - protein, 20 aa, Saccharomyces cerevisiae
MKFSTALSVALFALAKMVIA

SEQ ID NO: 56 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 57 - protein, 18 aa, Saccharomyces cerevisiae
MQYKKTLVASALAATTLA

SEQ ID NO: 58 - protein, 227 aa, Saccharomyces cerevisiae; identical to SEQ ID NO: 28

SEQ ID NO: 59 - protein, 19 aa, Saccharomyces cerevisiae
MSVSKIAFVLSAIASLAVA

SEQ ID NO: 60 - protein, 18 aa, Saccharomyces cerevisiae
MKLSTVLLSAGLASTTLA

SEQ ID NO: 61 - protein, 18 aa, Saccharomyces cerevisiae
MAYTKIALFAAIAALASA

SEQ ID NO: 62 - protein, 18 aa, Saccharomyces cerevisiae
MLEFPISVLLGCLVAVKA

SEQ ID NO: 63 - protein, 18 aa, Saccharomyces cerevisiae
MKFSTLSTVAAIAAFASA

SEQ ID NO: 64 - protein, 44 aa, Saccharomyces cerevisiae
MTKPTQVLVRSVSILFFITLLHLVVALNDVAGPAETAPVSLLPR

SEQ ID NO: 65 - protein, 19 aa, Saccharomyces cerevisiae
MSRISILAVAAALVASATA

SEQ ID NO: 66 - protein, 19 aa, Saccharomyces cerevisiae
MRFPSIFTAVLFAASSALA

SEQ ID NO: 67 - protein, 20 aa, Saccharomyces cerevisiae
MKAFTSLLCGLGLSTTLAKA

SEQ ID NO: 68 - protein, 23 aa, Saccharomyces cerevisiae
MFNRFNKLQAALALVLYSQSALG

SEQ ID NO: 69 - protein, 18 aa, Saccharomyces cerevisiae
MRFSNFLTVSALLTGALG

SEQ ID NO: 70 - protein, 18 aa, Saccharomyces cerevisiae
MISANSLLISTLCAFAIA

SEQ ID NO: 71 - protein, 19 aa, Saccharomyces cerevisiae
MFTFLKIILWLFSLALASA

SEQ ID NO: 72 - protein, 329 aa, Saccharomyces cerevisiae
YQGRNLGTASAKSSFISTTTTDLTSINTSAYSTGSISTVETGNRTTSEVISHVVTTSTKLSPTATTSLTIAQTSIYSTDSNITVGTDIHTTSEVISDVETISRETASTVVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 73 - protein, 321 aa, Saccharomyces cerevisiae
ASAKSSFISTTTTDLTSINTSAYSTGSISTVETGNRTTSEVISHVVTTSTKLSPTATTSLTIAQTSIYSTDSNITVGTDIHTTSEVISDVETISRETASTVVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 74 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 75 - protein, 312 aa, Saccharomyces cerevisiae
TTTTDLTSINTSAYSTGSISTVETGNRTTSEVISHVVTTSTKLSPTATTSLTIAQTSIYSTDSNITVGTDIHTTSEVISDVETISRETASTVVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 76 - protein, 301 aa, Saccharomyces cerevisiae
SAYSTGSISTVETGNRTTSEVISHVVTTSTKLSPTATTSLTIAQTSIYSTDSNITVGTDIHTTSEVISDVETISRETASTVVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 77 - protein, 291 aa, Saccharomyces cerevisiae
VETGNRTTSEVISHVVTTSTKLSPTATTSLTIAQTSIYSTDSNITVGTDIHTTSEVISDVETISRETASTVVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 78 - protein, 281 aa, Saccharomyces cerevisiae
VISHVVTTSTKLSPTATTSLTIAQTSIYSTDSNITVGTDIHTTSEVISDVETISRETASTVVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 79 - protein, 267 aa, Saccharomyces cerevisiae
TATTSLTIAQTSIYSTDSNITVGTDIHTTSEVISDVETISRETASTVVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 80 - protein, 261 aa, Saccharomyces cerevisiae
TIAQTSIYSTDSNITVGTDIHTTSEVISDVETISRETASTVVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 81 - protein, 251 aa, Saccharomyces cerevisiae
DSNITVGTDIHTTSEVISDVETISRETASTVVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 82 - protein, 241 aa, Saccharomyces cerevisiae
HTTSEVISDVETISRETASTVVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 83 - protein, 231 aa, Saccharomyces cerevisiae
ETISRETASTVVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 84 - protein, 221 aa, Saccharomyces cerevisiae
VVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 85 - protein, 211 aa, Saccharomyces cerevisiae
WTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 86 - protein, 201 aa, Saccharomyces cerevisiae
QFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 87 - protein, 191 aa, Saccharomyces cerevisiae
NSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 88 - protein, 181 aa, Saccharomyces cerevisiae
VFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 89 - protein, 171 aa, Saccharomyces cerevisiae
NVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 90 - protein, 161 aa, Saccharomyces cerevisiae
AAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 91 - protein, 151 aa, Saccharomyces cerevisiae
VNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 92 - protein, 141 aa, Saccharomyces cerevisiae
FCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 93 - protein, 131 aa, Saccharomyces cerevisiae
SSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 94 - protein, 121 aa, Saccharomyces cerevisiae
SLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 95 - protein, 111 aa, Saccharomyces cerevisiae
TSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 96 - protein, 101 aa, Saccharomyces cerevisiae
NTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 97 - protein, 91 aa, Saccharomyces cerevisiae
YFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 98 - protein, 81 aa, Saccharomyces cerevisiae
SVGLNSFSETAVSSQGTKIDTFLVSSLIAYPSSASGSQLSGIQQNFTSTSLMISTYEGKASIFFSAELGSIIFLLLSYLLF

SEQ ID NO: 99 - protein, 71 aa, Saccharomyces cerevisiae
AVSSQGTKIDTFLVSSLIAYPSSASGSQLSGIQQNFTSTSLMISTYEGKASIFFSAELGSIIFLLLSYLLF

SEQ ID NO: 100 - protein, 61 aa, Saccharomyces cerevisiae
TFLVSSLIAYPSSASGSQLSGIQQNFTSTSLMISTYEGKASIFFSAELGSIIFLLLSYLLF

SEQ ID NO: 101 - protein, 51 aa, Saccharomyces cerevisiae
PSSASGSQLSGIQQNFTSTSLMISTYEGKASIFFSAELGSIIFLLLSYLLF

SEQ ID NO: 102 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 103 - protein, 416 aa, Saccharomyces cerevisiae
YPSNGTSVISSSVISSSVTSSLVTSSSFISSSVISSSTTTSTSIFSESSTSSVIPTSSSTSGSSESKTSSASSSSSSSSISSESPKSPTNSSSSLPPVTSATTGQETASSLPPATTTKTSEQTTLVTVTSCESHVCTESISSAIVSTATVTVSGVTTEYTTWCPISTTETTKQTKGTTEQTKGTTEQTTETTKQTTVVTISSCESDICSKTASPAIVSTSTATINGVTTEYTTWCPISTTESKQQTTLVTVTSCESGVCSETTSPAIVSTATATVNDVVTVYPTWRPQTTNEQSVSSKMNSATSETTTNTGAAETKTAVTSSLSRFNHAETQTASATDVIGHNNSVVSVSETGNTKSLTSSGLSTMSQQPRSTPASSMVGYSTASLEISTYAGSANSLLAGSGLSVFIASLLLAII

SEQ ID NO: 104 - protein, 415 aa, Saccharomyces cerevisiae
PSNGTSVISSSVISSSVTSSLVTSSSFISSSVISSSTTTSTSIFSESSTSSVIPTSSSTSGSSESKTSSASSSSSSSSISSESPKSPTNSSSSLPPVTSATTGQETASSLPPATTTKTSEQTTLVTVTSCESHVCTESISSAIVSTATVTVSGVTTEYTTWCPISTTETTKQTKGTTEQTKGTTEQTTETTKQTTVVTISSCESDICSKTASPAIVSTSTATINGVTTEYTTWCPISTTESKQQTTLVTVTSCESGVCSETTSPAIVSTATATVNDVVTVYPTWRPQTTNEQSVSSKMNSATSETTTNTGAAETKTAVTSSLSRFNHAETQTASATDVIGHNNSVVSVSETGNTKSLTSSGLSTMSQQPRSTPASSMVGYSTASLEISTYAGSANSLLAGSGLSVFIASLLLAII

SEQ ID NO: 105 - protein, 414 aa, Saccharomyces cerevisiae
SNGTSVISSSVISSSVTSSLVTSSSFISSSVISSSTTTSTSIFSESSTSSVIPTSSSTSGSSESKTSSASSSSSSSSISSESPKSPTNSSSSLPPVTSATTGQETASSLPPATTTKTSEQTTLVTVTSCESHVCTESISSAIVSTATVTVSGVTTEYTTWCPISTTETTKQTKGTTEQTKGTTEQTTETTKQTTVVTISSCESDICSKTASPAIVSTSTATINGVTTEYTTWCPISTTESKQQTTLVTVTSCESGVCSETTSPAIVSTATATVNDVVTVYPTWRPQTTNEQSVSSKMNSATSETTTNTGAAETKTAVTSSLSRFNHAETQTASATDVIGHNNSVVSVSETGNTKSLTSSGLSTMSQQPRSTPASSMVGYSTASLEISTYAGSANSLLAGSGLSVFIASLLLAII

SEQ ID NO: 106 - protein, 413 aa, Saccharomyces cerevisiae
NGTSVISSSVISSSVTSSLVTSSSFISSSVISSSTTTSTSIFSESSTSSVIPTSSSTSGSSESKTSSASSSSSSSSISSESPKSPTNSSSSLPPVTSATTGQETASSLPPATTTKTSEQTTLVTVTSCESHVCTESISSAIVSTATVTVSGVTTEYTTWCPISTTETTKQTKGTTEQTKGTTEQTTETTKQTTVVTISSCESDICSKTASPAIVSTSTATINGVTTEYTTWCPISTTESKQQTTLVTVTSCESGVCSETTSPAIVSTATATVNDVVTVYPTWRPQTTNEQSVSSKMNSATSETTTNTGAAETKTAVTSSLSRFNHAETQTASATDVIGHNNSVVSVSETGNTKSLTSSGLSTMSQQPRSTPASSMVGYSTASLEISTYAGSANSLLAGSGLSVFIASLLLAII

SEQ ID NO: 107 - protein, 404 aa, Saccharomyces cerevisiae
VISSSVTSSLVTSSSFISSSVISSSTTTSTSIFSESSTSSVIPTSSSTSGSSESKTSSASSSSSSSSISSESPKSPTNSSSSLPPVTSATTGQETASSLPPATTTKTSEQTTLVTVTSCESHVCTESISSAIVSTATVTVSGVTTEYTTWCPISTTETTKQTKGTTEQTKGTTEQTTETTKQTTVVTISSCESDICSKTASPAIVSTSTATINGVTTEYTTWCPISTTESKQQTTLVTVTSCESGVCSETTSPAIVSTATATVNDVVTVYPTWRPQTTNEQSVSSKMNSATSETTTNTGAAETKTAVTSSLSRFNHAETQTASATDVIGHNNSVVSVSETGNTKSLTSSGLSTMSQQPRSTPASSMVGYSTASLEISTYAGSANSLLAGSGLSVFIASLLLAII

SEQ ID NO: 108 - protein, 394 aa, Saccharomyces cerevisiae
VTSSSFISSSVISSSTTTSTSIFSESSTSSVIPTSSSTSGSSESKTSSASSSSSSSSISSESPKSPTNSSSSLPPVTSATTGQETASSLPPATTTKTSEQTTLVTVTSCESHVCTESISSAIVSTATVTVSGVTTEYTTWCPISTTETTKQTKGTTEQTKGTTEQTTETTKQTTVVTISSCESDICSKTASPAIVSTSTATINGVTTEYTTWCPISTTESKQQTTLVTVTSCESGVCSETTSPAIVSTATATVNDVVTVYPTWRPQTTNEQSVSSKMNSATSETTTNTGAAETKTAVTSSLSRFNHAETQTASATDVIGHNNSVVSVSETGNTKSLTSSGLSTMSQQPRSTPASSMVGYSTASLEISTYAGSANSLLAGSGLSVFIASLLLAII

SEQ ID NO: 109 - protein, 384 aa, Saccharomyces cerevisiae
VISSSTTTSTSIFSESSTSSVIPTSSSTSGSSESKTSSASSSSSSSSISSESPKSPTNSSSSLPPVTSATTGQETASSLPPATTTKTSEQTTLVTVTSCESHVCTESISSAIVSTATVTVSGVTTEYTTWCPISTTETTKQTKGTTEQTKGTTEQTTETTKQTTVVTISSCESDICSKTASPAIVSTSTATINGVTTEYTTWCPISTTESKQQTTLVTVTSCESGVCSETTSPAIVSTATATVNDVVTVYPTWRPQTTNEQSVSSKMNSATSETTTNTGAAETKTAVTSSLSRFNHAETQTASATDVIGHNNSVVSVSETGNTKSLTSSGLSTMSQQPRSTPASSMVGYSTASLEISTYAGSANSLLAGSGLSVFIASLLLAII

SEQ ID NO: 110 - protein, 374 aa, Saccharomyces cerevisiae
SIFSESSTSSVIPTSSSTSGSSESKTSSASSSSSSSSISSESPKSPTNSSSSLPPVTSATTGQETASSLPPATTTKTSEQTTLVTVTSCESHVCTESISSAIVSTATVTVSGVTTEYTTWCPISTTETTKQTKGTTEQTKGTTEQTTETTKQTTVVTISSCESDICSKTASPAIVSTSTATINGVTTEYTTWCPISTTESKQQTTLVTVTSCESGVCSETTSPAIVSTATATVNDVVTVYPTWRPQTTNEQSVSSKMNSATSETTTNTGAAETKTAVTSSLSRFNHAETQTASATDVIGHNNSVVSVSETGNTKSLTSSGLSTMSQQPRSTPASSMVGYSTASLEISTYAGSANSLLAGSGLSVFIASLLLAII

SEQ ID NO: 111 - protein, 364 aa, Saccharomyces cerevisiae
VIPTSSSTSGSSESKTSSASSSSSSSSISSESPKSPTNSSSSLPPVTSATTGQETASSLPPATTTKTSEQTTLVTVTSCESHVCTESISSAIVSTATVTVSGVTTEYTTWCPISTTETTKQTKGTTEQTKGTTEQTTETTKQTTVVTISSCESDICSKTASPAIVSTSTATINGVTTEYTTWCPISTTESKQQTTLVTVTSCESGVCSETTSPAIVSTATATVNDVVTVYPTWRPQTTNEQSVSSKMNSATSETTTNTGAAETKTAVTSSLSRFNHAETQTASATDVIGHNNSVVSVSETGNTKSLTSSGLSTMSQQPRSTPASSMVGYSTASLEISTYAGSANSLLAGSGLSVFIASLLLAII

SEQ ID NO: 112 - protein, 354 aa, Saccharomyces cerevisiae
SSESKTSSASSSSSSSSISSESPKSPTNSSSSLPPVTSATTGQETASSLPPATTTKTSEQTTLVTVTSCESHVCTESISSAIVSTATVTVSGVTTEYTTWCPISTTETTKQTKGTTEQTKGTTEQTTETTKQTTVVTISSCESDICSKTASPAIVSTSTATINGVTTEYTTWCPISTTESKQQTTLVTVTSCESGVCSETTSPAIVSTATATVNDVVTVYPTWRPQTTNEQSVSSKMNSATSETTTNTGAAETKTAVTSSLSRFNHAETQTASATDVIGHNNSVVSVSETGNTKSLTSSGLSTMSQQPRSTPASSMVGYSTASLEISTYAGSANSLLAGSGLSVFIASLLLAII

SEQ ID NO: 113 - protein, 6 aa, synthetic construct
GSGGSG

SEQ ID NO: 114 - protein, 6 aa, synthetic construct
GSGSGS

SEQ ID NO: 115 - protein, 10 aa, synthetic construct
HHHHGSGGSG

SEQ ID NO: 116 - protein, 12 aa, synthetic construct
GSGAGGVSGAGG

SEQ ID NO: 117 - protein, 12 aa, synthetic construct
GSGGSGGSGGSG

SEQ ID NO: 118 - protein, 12 aa, synthetic construct
HHHHHHGSGGSG

SEQ ID NO: 119 - protein, 18 aa, synthetic construct
GSGGSGGSGGSGGSGGSG

SEQ ID NO: 120 - protein, 12 aa, synthetic construct
AEAAAKEAAAKA

SEQ ID NO: 121 - protein, 15 aa, synthetic construct
APAPAPAPAPAPAPA

SEQ ID NO: 122 - protein, 15 aa, synthetic construct
EPEPEPEPEPEPEPE

SEQ ID NO: 123 - protein, 14 aa, synthetic construct
KPKPKPKPKPKPKP

SEQ ID NO: 124 - protein, 17 aa, synthetic construct
AEAAAKEAAAKEAAAKA

SEQ ID NO: 125 - protein, 22 aa, synthetic construct
AEAAAKEAAAKEAAAKEAAAKA

SEQ ID NO: 126 - protein, 20 aa, Saccharomyces cerevisiae
MQLLRCFSIFSVIASVLARR

SEQ ID NO: 127 - protein, 26 aa, Saccharomyces cerevisiae
MSAINHLCLKLILASFAIINTITARR

SEQ ID NO: 128 - protein, 24 aa, Saccharomyces cerevisiae
MRQVWFSWIVGLFLCFFNVSSARR

SEQ ID NO: 129 - protein, 24 aa, Saccharomyces cerevisiae
MFSLKALLPLALLLVSANQVAARR

SEQ ID NO: 130 - protein, 20 aa, Saccharomyces cerevisiae
MQYKKSLVASALVATSLARR

SEQ ID NO: 131 - protein, 20 aa, Saccharomyces cerevisiae
MQYKKPLVVSALAATSLARR

SEQ ID NO: 132 - protein, 21 aa, Saccharomyces cerevisiae
MFTFLKIILWLFSLALASARR

SEQ ID NO: 133 - protein, 21 aa, Saccharomyces cerevisiae
MLLQAFLFLLAGFAAKISARR

SEQ ID NO: 134 - protein, 540 aa, Cannabis sativa
TFSFWFVCKIIFFFFSFNIQTSIANPRENFLKCFSQYIPNNATNLKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYWVNEKNESLSLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHRH

SEQ ID NO: 135 - protein, 539 aa, Cannabis sativa
FSFWFVCKIIFFFFSFNIQTSIANPRENFLKCFSQYIPNNATNLKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYWVNEKNESLSLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHRH

SEQ ID NO: 136 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYWVNEKNESLSLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHRH

SEQ ID NO: 137 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLTLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKTFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHRH

SEQ ID NO: 138 - protein, 22 aa, Saccharomyces cerevisiae
MQFSTVASVAFVALANFVAARR

SEQ ID NO: 139 - protein, 22 aa, Saccharomyces cerevisiae
MQFSTVASVAFVALANFVAAKR

SEQ ID NO: 140 - protein, 23 aa, Saccharomyces cerevisiae
MQFSTVASVAFVALANFVAARRK

SEQ ID NO: 141 - protein, 23 aa, Saccharomyces cerevisiae
MQFSTVASVAFVALANFVAARRQ

SEQ ID NO: 142 - protein, 23 aa, Saccharomyces cerevisiae
MQFSTVASVAFVALANFVAARRW

SEQ ID NO: 143 - protein, 23 aa, Saccharomyces cerevisiae
MQFSTVASVAFVALANFVAARRE

SEQ ID NO: 144 - protein, 24 aa, Saccharomyces cerevisiae
MQFSTVASVAFVALANFVAALDKR

SEQ ID NO: 145 - protein, 28 aa, Saccharomyces cerevisiae
MQFSTVASVAFVALANFVAALDKREAEA

SEQ ID NO: 146 - protein, 26 aa, Saccharomyces cerevisiae
MQFSTVASVAFVALANFVAAKREAEA

SEQ ID NO: 147 - protein, 521 aa, Cannabis sativa
QTSIANPRENFLKCFSQYIPNNATNLKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYWVNEKNESLSLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHRH

SEQ ID NO: 148 - protein, 544 aa, Cannabis sativa
MKCSTFSFWFVCKIIFFFFSFNIQTSIANPTENFLKCFSQYIDNNATNDKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYNVNEKNENLTLAAGYCPTVCAGGHFGGGGYGPLMRSYGLAADNIIDAHLVNVDGKVLDRKSMGEDLFWALRGGGAESFGIVVAWKIRLVAVPKSTMFSVKKIMEIHELVKLVNKWQNIAYKYDKDLLLMTHFITRNITDNQGNNKTAIHTYFSCVFLGGVDSLVDLMNKTFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHRH

SEQ ID NO: 149 - protein, 516 aa, Cannabis sativa
NPRENFLKCFSQYIDNNATNDKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYNVNEKNENLTLAAGYCPTVCAGGHFGGGGYGPLMRSYGLAADNIIDAHLVNVDGKVLDRKSMGEDLFWALRGGGAESFGIVVAWKIRLVAVPKSTMFSVKKIMEIHELVKLVNKWQNIAYKYDKDLLLMTHFITRNITDNQGNNKTAIHTYFSCVFLGGVDSLVDLMNKTFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHRH

SEQ ID NO: 150 - protein, 543 aa, Cannabis sativa
MKCSTFSFWFVCKIIFFFFSFNIQTSIANPTENFLKCFSQYIDNNATNDKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYNVNEKNENLTLAAGYCPTVCAGGHFGGGGYGPLMRSYGLAADNIIDAHLVNVDGKVLDRKSMGEDLFWALRGGGAESFGIVVAWKIRLVAVPKSTMFSVKKIMEIHELVKLVNKWQNIAYKYDKDLLLMTHFITRNITDNQGNNKTAIHTYFSCVFLGGVDSLVDLMNKTFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHR

SEQ ID NO: 151 - protein, 515 aa, Cannabis sativa
NPRENFLKCFSQYIDNNATNDKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYNVNEKNENLTLAAGYCPTVCAGGHFGGGGYGPLMRSYGLAADNIIDAHLVNVDGKVLDRKSMGEDLFWALRGGGAESFGIVVAWKIRLVAVPKSTMFSVKKIMEIHELVKLVNKWQNIAYKYDKDLLLMTHFITRNITDNQGNNKTAIHTYFSCVFLGGVDSLVDLMNKTFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHR

SEQ ID NO: 152 - protein, 7 aa, synthetic construct
VVPAIPN

SEQ ID NO: 153 - protein, 89 aa, Saccharomyces cerevisiae
MRFPSIFTAVLFAASSALAAPVNTTTEDETAQIPAEAVIGYLDLEGDFDVAVLPFSNSTNNGLLFINTTIASIAAKEEGVSLDKREAEA

SEQ ID NO: 154 - protein, 72 aa, Saccharomyces cerevisiae
MRFPSIFTAVLFAASSALAQPIDDTESNTTSVNLMADDTESRFATNTTLALDVVNLISMAKREEAEAEAEPK

SEQ ID NO: 155 - protein, 69 aa, Saccharomyces cerevisiae
MRFPSIFTAVLFAASSALAQPIDDTESQTTSVNLMADDTESAFATQTNSGGLDVVGLISMAKREEGEPK

SEQ ID NO: 156 - protein, 417 aa, Saccharomyces cerevisiae
MFSLKALLPLALLLVSANQVAAKVHKAKIYKHELSDEMKEVTFEQHLAHLGQKYLTQFEKANPEVVFSREHPFFTEGGHDVPLTNYLNAQYYTDITLGTPPQNFKVILDTGSSNLWVPSNECGSLACFLHSKYDHEASSSYKANGTEFAIQYGTGSLEGYISQDTLSIGDLTIPKQDFAEATSEPGLTFAFGKFDGILGLGYDTISVDKVVPPFYNAIQQDLLDEKRFAFYLGDTSKDTENGGEATFGGIDESKFKGDITWLPVRRKAYWEVKFEGIGLGDEYAELESHGAAIDTGTSLITLPSGLAEMINAEIGAKKGWTGQYTLDCNTRDNLPDLIFNFNGYNFTIGPYDYTLEVSGSCISAITPMDFPEPVGPLAIVGDAFLRKYYSIYDLGNNAVGLAKAIAEAAAKEAAAKA

SEQ ID NO: 157 - protein, 88 aa, Saccharomyces cerevisiae
MFSLKALLPLALLLVSANQVAAKVHKAKIYKHELSDEMKEVTFEQHLAHLGQKYLTQFEKANPEVVFSREHPFFTEAEAAAKEAAAKA

SEQ ID NO: 158 - DNA, 500 bp, synthetic construct
  source 1..500: mol_type = other DNA
tggaactttcagtaatacgcttaactgctcattgctatattgaagtacggattagaagccgccgagcgggcgacagccctccgacggaagactctcctccgtgcgtcctggtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactttaacgtcaaggagaaaaaactata

SEQ ID NO: 159 - DNA, 501 bp, synthetic construct
  source 1..501: mol_type = other DNA
catcgcttcgctgattaattaccccagaaataaggctaaaaaactaatcgcattattatcctatggttgttaatttgattcgttgatttgaaggtttgtggggccaggttactgccaatttttcctcttcataaccataaaagctagtattgtagaatctttattgttcggagcagtgcggcgcgaggcacatctgcgtttcaggaacgcgaccggtgaagaccaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtattgccatgtaatatgattattaaacttctttgcgtccatccaaaaaaaaagtaagaatttttgaaaattcaatataa

SEQ ID NO: 160 - DNA, 509 bp, synthetic construct
  source 1..509: mol_type = other DNA
ggcttaagtaggttgcaatttctttttctattagtagctaaaaatgggtcacgtgatctatattcgaaaggggcggttgcctcaggaaggcaccggcggtctttcgtccgtgcggagatatctgcgccgttcaggggtccatgtgccttggacgatattaaggcagaaggcagtatcggggcggatcactccgaaccgagattagttaagcccttcccatctcaagatggggagcaaatggcattatactcctgctagaaagttaactgtgcacatattcttaaattatacaatgttctggagagctattgtttaaaaaacaaacatttcgcaggctaaaatgtggagataggattagttttgtagacatatataaacaatcagtaattggattgaaaatttggtgttgtgaattgctcttcattatgcaccttattcaattatcatcaagaatagcaatagttaagtaaacacaagattaacataataaaaaaaataattctttcata

SEQ ID NO: 161 - DNA, 498 bp, synthetic construct
  source 1..498: mol_type = other DNA
ttttactattatcttctacgctgacagtaatatcaaacagtgacacatattaaacacagtggtttctttgcataaacaccatcagcctcaagtcgtcaagtaaagatttcgtgttcatgcagatagataacaatctatatgttgataattagcgttgcctcatcaatgcgagatccgtttaaccggaccctagtgcacttaccccacgttcggtccactgtgtgccgaacatgctccttcactattttaacatgtggaattcttgaaagaatgaaatcgccatgccaagccatcacacggtcttttatgcaattgattgaccgcctgcaacacataggcagtaaaatttttactgaaacgtatataatcatcataagcgacaagtgaggcaacacctttgttaccacattgacaaccccaggtattcatacttcctattagcggaatcaggagtgcaaaaagagaaaataaaagtaaaaaggtagggcaacacatagt

SEQ ID NO: 162 - DNA, 501 bp, synthetic construct
  source 1..501: mol_type = other DNA
ggacggtagcaacaagaatatagcacgagccgcgaagttcatttcgttacttttgatatcgctcacaactattgcgaagcgcttcagtgaaaaaatcataaggaaaagttgtaaatattattggtagtattcgtttggtaaagtagagggggtaatttttcccctttattttgttcatacattcttaaattgctttgcctctccttttggaaagctatacttcggagcactgttgagcgaaggctcattagatatattttctgtcattttccttaacccaaaaataagggaaagggtccaaaaagcgctcggacaactgttgaccgtgatccgaaggactggctatacagtgttcacaaaatagccaagctgaaaataatgtgtagctatgttcagttagtttggctagcaaagatataaaagcaggtcggaaatatttatgggcattattatgcagagcatcaacatgataaaaaaaaacagttgaatattccctcaaaa

SEQ ID NO: 163 - DNA, 489 bp, synthetic construct
  source 1..489: mol_type = other DNA
gcgacacagagatgacagacggtggcgcaggatccggtttaaacgaggatcccttaagtttaaacaacaacagcaagcaggtgtgcaagacactagagactcctaacatgatgtatgccaataaaacacaagagataaacaacattgcatggaggccccagaggggcgattggtttgggtgcgtgagcggcaagaagtttcaaaacgtccgcgtcctttgagacagcattcgcccagtattttttttattctacaaaccttctataatttcaaagtatttacataattctgtatcagtttaatcaccataatatcgttttctttgtttagtgcaattaatttttcctattgttacttcgggcctttttctgttttatgagctattttttccgtcatccttccccagattttcagcttcatctccagattgtgtctacgtaatgcacgccatcattttaagagaggacagagaagcaagcctcctgaaag

SEQ ID NO: 164 - DNA, 1253 bp, synthetic construct
  source 1..1253: mol_type = other DNA
gatgatggacactagtgtgtcgagaatgtatcaactatatatagtcctaatgccacacaaatatgaagtgggggaagcccattcttaatccggctcaattttggtgcgtgatcgcggcctatgtttgcttccagaaaaagcttagaataatatttctcacctttgatggaatgctcgcgagtgctcgttttgattaccccatatgcattgttgcagcatgcaagcactattgcaagccacgcatggaagaaatttgcaaacacctatagccccgcgttgttgaggaggtggacttggtgtaggaccataaagctgtgcactactatggtgagctctgtcgtctggtgaccttctatctcaggcacatcctcgtttttgtgcatgaggttcgagtcacgcccacggcctattaatccgcgaaataaatgcgaaatctaaattatgacgcaaggctgagagattctgacacgccgcatttgcggggcagtaattatcgggcagttttccggggttcgggatggggtttggagagaaagttcaacacagaccaaaacagcttgggaccacttggatggaggtccccgcagaagagctctggcgcgttggacaaacattgacaatccacggcaaaattgtctacagttccgtgtatgcggatagggatatcttcgggagtatcgcaataggatacaggcactgtgcagattacgcgacatgatagctttgtatgttctacagactctgccgtagcagtctagatataatatcggagttttgtagcgtcgtaaggaaaacttgggttacacaggtttcttgagagccctttgacgttgattgctctggcttccatccaggccctcatgtggttcaggtgcctccgcagtggctggcaagcgtgggggtcaattacgtcacttctattcatgtaccccagactcaattgttgacagcaatttcagcgagaattaaattccacaatcaattctcgctgaaataattaggccgtgatttaattctcgctgaaacagaatcctgtctggggtacagataacaatcaagtaactattatggacgtgcataggaggtggagtccatgacgcaaagggaaatattcattttatcctcgcgaagttgggatgtgtcaaagcgtcgcgctcgctatagtgatgagaatgtctttagtaagcttaagccatataaagaccttccgcctccatatttttttttatccctcttgacaatattaattcctt

SEQ ID NO: 165 - DNA, 1253 bp, synthetic construct
  source 1..1253: mol_type = other DNA
aaggaattaatattgtcaagagggataaaaaaaaatatggaggcggaaggtctttatatggcttaagcttactaaagacattctcatcactatagcgagcgcgacgctttgacacatcccaacttcgcgaggataaaatgaatatttccctttgcgtcatggactccacctcctatgcacgtccataatagttacttgattgttatctgtaccccagacaggattctgtttcagcgagaattaaatcacggcctaattatttcagcgagaattgattgtggaatttaattctcgctgaaattgctgtcaacaattgagtctggggtacatgaatagaagtgacgtaattgacccccacgcttgccagccactgcggaggcacctgaaccacatgagggcctggatggaagccagagcaatcaacgtcaaagggctctcaagaaacctgtgtaacccaagttttccttacgacgctacaaaactccgatattatatctagactgctacggcagagtctgtagaacatacaaagctatcatgtcgcgtaatctgcacagtgcctgtatcctattgcgatactcccgaagatatccctatccgcatacacggaactgtagacaattttgccgtggattgtcaatgtttgtccaacgcgccagagctcttctgcggggacctccatccaagtggtcccaagctgttttggtctgtgttgaactttctctccaaaccccatcccgaaccccggaaaactgcccgataattactgccccgcaaatgcggcgtgtcagaatctctcagccttgcgtcataatttagatttcgcatttatttcgcggattaataggccgtgggcgtgactcgaacctcatgcacaaaaacgaggatgtgcctgagatagaaggtcaccagacgacagagctcaccatagtagtgcacagctttatggtcctacaccaagtccacctcctcaacaacgcggggctataggtgtttgcaaatttcttccatgcgtggcttgcaatagtgcttgcatgctgcaacaatgcatatggggtaatcaaaacgagcactcgcgagcattccatcaaaggtgagaaatattattctaagctttttctggaagcaaacataggccgcgatcacgcaccaaaattgagccggattaagaatgggcttcccccacttcatatttgtgtggcattaggactatatatagttgatacattctcgacacactagtgtccatcatc

SEQ ID NO: 166 - DNA, 665 bp, synthetic construct
  source 1..665: mol_type = other DNA
gcgcctcaagaaaatgatgctgcaagaagaattgaggaaggaactattcatcttacgttgtttgtatcatcccacgatccaaatcatgttacctacgttaggtacgctaggaactaaaaaaagaaaagaaaagtatgcgttatcactcttcgagccaattcttaattgtgtggggtccgcgaaaatttccggataaatcctgtaaactttaacttaaaccccgtgtttagcgaaattttcaacgaagcgcgcaataaggagaaatattatctaaaagcgagagtttaagcgagttgcaagaatctctacggtacagatgcaacttactatagccaaggtctattcgtattactatggcagcgaaaggagctttaaggttttaattaccccatagccatagattctactcggtctatctatcatgtaacactccgttgatgcgtactagaaaatgacaacgtaccgggcttgagggacatacagagacaattacagtaatcaagagtgtacccaactttaacgaactcagtaaaaaataaggaatgtcgacatcttaattttttatataaagcggtttggtattgattgtttgaagaattttcgggttggtgtttctttctgatgctacatagaagaacatcaaacaactaaaaaaatagtataat

SEQ ID NO: 167 - DNA, 785 bp, synthetic construct
  source 1..785: mol_type = other DNA
attatactatttttttagttgtttgatgttcttctatgtagcatcagaaagaaacaccaacccgaaaattcttcaaacaatcaataccaaaccgctttatataaaaaattaagatgtcgacattccttattttttactgagttcgttaaagttgggtacactcttgattactgtaattgtctctgtatgtccctcaagcccggtacgttgtcattttctagtacgcatcaacggagtgttacatgatagatagaccgagtagaatctatggctatggggtaattaaaaccttaaagctcctttcgctgccatagtaatacgaatagaccttggctatagtaagttgcatctgtaccgtagagattcttgcaactcgcttaaactctcgcttttagataatatttctccttattgcgcgcttcgttgaaaatttcgctaaacacggggtttaagttaaagtttacaggatttatccggaaattttcgcggaccccacacaattaagaattggctcgaagagtgataacgcatacttttcttttctttttttagttcctagcgtacctaacgtaggtaacatgatttggatcgtgggatgatacaaacaacgtaagatgaatagttccttcctcaattcttcttgcagcatcattttcttgaggcgctctgggcaaggtataaaaagttccattaatacgtctctaaaaaattaaatcatccatctcttaagcagtttttttgataatctcaaatgtacatcagtcaagcgtaactaaattacataa

SEQ ID NO: 168 - DNA, 875 bp, synthetic construct
  source 1..875: mol_type = other DNA
ttatgtattttagttacgcttgactgatgtacatttgagattatcaaaaaaactgcttaagagatagatggtttaattttttagagacgtattaatggaactttttataccttgcccagagcgcctcaagaaaatgatgctgaaagaagaattgaggaaggaactactcatcttacgttgtttgtatcatcccacgatccaaatcatgttacctacgttaggtacgctaggaactgaaaaaagaaaagaaaagtatgcgttatcactcttcgagccaattcttaattgtgtggggtccgcgaaaacttccggataaatcctgtaaacttaaacttaaaccccgtgtttagcgaaattttcaacgaagcgcgcaataaggagaaatattatataaaagcgagagtttaagcgaggttgcaagaatctctacggtacagatgcaacttactatagccaaggtctattcgtattggtatccaagcagtgaagctactcaggggaaaacatattttcagagatcaaagttatgtcagtctctttttcatgtgtaacttaacgtttgtgcaggtatcataccggcctccacataatttttgtggggaagacgttgttgtagcagtctccttatactctccaacaggtgtttaaagacttcttcaggcctcatagtctacatctggagacaacattagatagaagtttccacagaggcagctttcaatatactttcggctgtgtacatttcatcctgagtgagcgcatattgcataagtactcagtatataaagagacacaatatactccatacttgttgtgagtggttttagcgtattcagtataacaataagaattacatccaagactattaattaact

SEQ ID NO: 169 - DNA, 875 bp, synthetic construct
  source 1..875: mol_type = other DNA
agttaattaatagtcttggatgtaattcttattgttatactgaatacgctaaaaccactcacaacaagtatggagtatattgtgtctctttatatactgagtacttatgcaatatgcgctcactcaggatgaaatgtacacagccgaaagtatattgaaagctgcctctgtggaaacttctatctaatgttgtctccagatgtagactatgaggcctgaagaagtctttaaacacctgttggagagtataaggagactgctacaacaacgtcttccccacaaaaattatgtggaggccggtatgatacctgcacaaacgttaagttacacatgaaaaagagactgacataactttgatctctgaaaatatgttttcccctgagtagcttcactgcttggataccaatacgaatagaccttggctatagtaagttgcatctgtaccgtagagattcttgcaacctcgcttaaactctcgcttttatataatatttctccttattgcgcgcttcgttgaaaatttcgctaaacacggggtttaagtttaagtttacaggatttatccggaagttttcgcggaccccacacaattaagaattggctcgaagagtgataacgcatacttttcttttcttttttcagttcctagcgtacctaacgtaggtaacatgatttggatcgtgggatgatacaaacaacgtaagatgagtagttccttcctcaattcttctttcagcatcattttcttgaggcgctctgggcaaggtataaaaagttccattaatacgtctctaaaaaattaaaccatctatctcttaagcagtttttttgataatctcaaatgtacatcagtcaagcgtaactaaaatacataa

SEQ ID NO: 170 - protein, 329 aa, Stachybotrys chartarum
MSAKVSPMAYTNPRYETGPLSLIPKPIVPYFELMRFELPHGYYLGYFPHLVGIMYGASAGPERLPARDLVFQALLYVGWTFAMRGAGCAWNDNIDQDFDRKTERCRTRPIARGAVSTTAGHVFAVAGVALAFLCLSPLPTECHQLGVLFTVLSVIYPFCKRFTNFAQVILGMTLAANFILAAYGAGLPALEQPYTRPTMSATLAITLLVVFYDVVYARQDTADDLKSGVKGMAVLFRNHIEVLLAVLTCTIGGLLAATGVSVGNGPYYFLFSVAGLTVALLAMIGGIRYRIFHTWNGYSGWFYVLAIINLMSGYFIEYLDNAPILARGS

SEQ ID NO: 171 - protein, 357 aa, Streptomyces aculeolatus
MTTEVTSFTGAGPHPAASVRRITDDLLQRVEDKLASFLTAERDRYAAMDERALAAVDALTDLVTSGGKRVRPTFCITGYLAAGGDAGDPGIVAAAAGLEMLHVSALIHDDILDNSAQRRGKPTIHTLYGDLHDSHGWRGESRRFGEGIGILIGNLALVYSQELVCQAPPAVLAEWHRLCSEVNIGQCLDVCAAAEFSADPELSRLVALIKSGRYTIHRPLVMGANAASRPDLAAAYVEYGEAVGEAFQLRDDLLDAFGDSTETGKPTGLDFTQHKMTLLLGWAMQRDTHIRTLMTEPGHTPEEVRRRLEDTEVPKDVERHIADLVEQGRAAIADAPIDPQWRQELADMAVRAAYRTN

SEQ ID NO: 172 - protein, 29 aa, synthetic construct
EPEPEPEPEPEPEPEASAKALLSQPLLLI

(19) United States
(12) Patent Application Publication
(10) Pub. No.: US 2024/0344093 A1
(43) Pub. Date: Oct. 17, 2024

(54) HIGH EFFICIENCY PRODUCTION OF CANNABIDIOLIC ACID

(71) Applicant: AMYRIS, INC., Emeryville, CA (US)

(72) Inventors: John E. Hung, Emeryville, CA (US); William E. Draper, Emeryville, CA (US); Victor Holmes, Emeryville, CA (US)

(21) Appl. No.: 18/578,649

(22) PCT Filed: Jul. 11, 2022
(86) PCT No.: PCT/US2022/073586

§ 371 Date: Jan. 11, 2024

Related U.S. Application Data
(60) Provisional application No. 63/221,173, filed on Jul. 13, 2021.

(51) Int. Cl. C12P 7/42; C12N 9/02; C12N 15/62; C12N 15/81; C12P 7/22

(52) U.S. Cl. CPC C12P 7/42; C12N 9/0004; C12N 15/62; C12N 15/81; C12P 7/22; C07K 2319/02; C07K 2319/50; C12Y 121/03008

(57) ABSTRACT

The present disclosure features compositions and methods for producing one or more cannabinoids, such as cannabidiolic acid (CBDa), in a host cell, such as a yeast cell, that is genetically modified to express the enzymes of a cannabinoid biosynthetic pathway. Using the compositions and methods of the present invention, the host cell may be genetically modified to express one or more enzymes of a cannabinoid biosynthetic pathway, such as an enzyme having CBDa synthase (CBDaS) activity.

### BACKGROUND OF THE INVENTION

Cannabinoids are a group of structurally related molecules defined by their ability to interact with a distinct class of receptors (cannabinoid receptors). Both naturally occurring and synthetic cannabinoids are known. Naturally occurring cannabinoids are produced primarily by the Cannabis family of plants and include cannabigerol (CBG), cannabichromene (CBC), cannabidiol (CBD), cannabinol (CBN), cannabinodiol (CBDL), cannabicyclol (CBL), cannabielsoin (CBE), cannabitriol (CBT), tetrahydrocannabinol (THC), and tetrahydrocannabinolic acid (THCa). An expanding set of synthetic variants of cannabinoids have been designed to mimic the effects of the naturally occurring molecules.

Cannabinoids may be used to improve various aspects of human health. However, producing cannabinoids in preparative amounts and in high yield has been challenging. There remains a need for compositions and methods capable of preparing cannabinoids with high efficiency and chemical selectivity.

### SUMMARY OF THE INVENTION

Provided herein are compositions and methods for the improved production of a cannabinoid, such as cannabidiolic acid (CBDa), in a host cell, such as a yeast cell. For example, using the compositions and methods described herein, a host cell may be modified to express one or more enzymes of a cannabinoid biosynthetic pathway, such as an acyl-activating enzyme (AAE), a tetraketide synthase (TKS), a cannabigerolic acid synthase (CBGaS), a geranyl pyrophosphate (GPP) synthase, and/or a CBDa synthase (CBDaS). The host cell may then be cultured in a medium, for example, in the presence of an agent that regulates expression of the one or more enzymes. The host cell may be incubated for a time sufficient to allow for biochemical synthesis of a cannabinoid, for example cannabidiolic acid (CBDa), and the cannabinoid may then be separated from the host cell or from the medium.

In one aspect the invention provides for a genetically modified host cell capable of producing CBDa or CBD, wherein the genetically modified host cell contains one or more heterologous nucleic acids that each, independently, encodes an enzyme having CBDaS activity. In one embodiment the enzyme having CBDaS activity is a fusion protein. In another embodiment the fusion protein has an amino acid sequence of a CBDaS or a portion thereof. In further embodiments the fusion protein has an amino acid sequence that is at least 80% identical to the amino acid sequence of SEQ ID NOS: 1, 4, 7, 8, 9, 10, 11, 13, 15, 17, 19, 21, 4, 7, 8, 9, 10, 11, 13, 15, 17, 19, 21, 134, 135, 136, 137, 147, 148, 149, 150, or 151.

In yet additional embodiments the fusion protein comprises an amino acid sequence of a carrier protein or a portion thereof. In further embodiments the fusion protein has an amino acid sequence that is at least 80% identical to the amino acid sequence of SEQ ID NOS: 34, 36, 73, 77, 81, 87, 89, 90, 91, 103, 104, 105, 106, 107, 108, 109, 110, 111, or 112. In yet another embodiment the fusion protein has an amino acid sequence of a signal sequence or a portion thereof. In an embodiment the fusion protein has an amino acid sequence that is at least 80% identical to the amino acid sequence of SEQ ID NOS: 42, 43, 44, 45, 46, 47, 48, 49, 50, 51, 52, 53, or 54. In preferred embodiments the fusion protein has an amino acid sequence of a linker or a portion thereof. In yet another embodiment the fusion protein has an amino acid sequence that is at least 80% identical to the amino acid sequence of SEQ ID NOS: 113, 114, 115, 116, 117, 118, 119, 120, 121, 122, 123, 124, 125, 152, or 172. In an embodiment of the invention the fusion protein contains an amino acid sequence of a protease recognition site. In further embodiments the protease recognition site is RR, KR, RRK, RRQ, RRW, RRE, LDKR, LDKREAEA, or KREAEA. In yet another embodiment the fusion protein contains an amino acid sequence of a mating factor alpha (MFα) or a portion thereof. In additional embodiments the fusion protein has an amino acid sequence that is at least 80% identical to the amino acid sequence of SEQ ID NOS: 153, 154, 155, 156, or 157.

In preferred embodiments the fusion protein has two or more of: an amino acid sequence of a CBDaS or a portion thereof, an amino acid sequence that is at least 80% identical to the amino acid sequence of SEQ ID NOS: 1, 4, 7, 8, 9, 10, 11, 13, 15, 17, 19, 21, 4, 7, 8, 9, 10, 11, 13, 15, 17, 19, 21, 134, 135, 136, 137, 147, 148, 149, 150, or 151; an amino acid sequence of a carrier protein or a portion thereof; an amino acid sequence that is at least 80% identical to the amino acid sequence of SEQ ID NOS: 34, 36, 73, 77, 81, 87, 89, 90, 91, 103, 104, 105, 106, 107, 108, 109, 110, 111, or 112: an amino acid sequence of a signal sequence or a portion thereof; an amino acid sequence that is at least 80% identical to the amino acid sequence of SEQ ID NOS: 42, 43, 44, 45, 46, 47, 48, 49, 50, 51, 52, 53, or 54; an amino acid sequence of a linker or a portion thereof; an amino acid sequence at least 80% identical to the amino acid sequence of SEQ ID NOS: 113, 114, 115, 116, 117, 118, 119, 120, 121, 122, 123, 124, 125, 152, or 172; an amino acid sequence of a protease recognition site; a protease recognition site having the amino acid sequence RR, KR, RRK, RRQ, RRW, RRE, LDKR, LDKREAEA, or KREAEA; an amino acid sequence of a mating factor alpha (MFα) or a portion thereof; or an amino acid sequence that is at least 80% identical to the amino acid sequence of SEQ ID NOS: 153, 154, 155, 156, or 157.

In an embodiment of the invention the enzyme having CBDaS activity or the amino acid sequence of a CBDaS or a portion thereof contains one or more of the following mutations when aligned with and in reference to SEQ ID NO: 136: N45Q, N65Q, S168N, N296Q, N304Q, N328Q, N498Q, T47S, T67S, S170T, T298S, T306S, S330T, or T500S. In another embodiment the enzyme having CBDaS activity or the amino acid sequence of a CBDaS or a portion thereof comprises one or more amino acid substitutions occurring at position(s) 29, 31, 43, 49, 53, 56, 57, 65, 71, 78, 79, 93, 95, 103, 117, 125, 129, 143, 147, 151, 161, 183, 213, 235, 241, 263, 264, 285, 286, 303, 314, 325, 336, 339, 396, 436, 518, or 540 when aligned with and in reference to SEQ ID NO: 137. In yet another embodiment the one or more amino acid substitutions is: N29G, R31T, P43D, L49D, R53T, N56D, N57D, P65D, L71D, L7IS, N78D, N79D, L93D, G95A, V103Y, G117A, V125D, 1129L, H143A, V147D, 1151L, W16IR, W161A, W16IN, W161S, W161T, W161D, W161H, W183N, H213D, H213N, H235D, 1241V, 1263V, E264P, D285N, K303N, S314C, K325N, S336C, T339S, F396L, A436G, V518C, or V540C.

In a preferred embodiment of the invention the enzyme having CBDaS activity or the amino acid sequence of a CBDaS or a portion thereof has one or more sets of the following amino acid substitutions: R53T, N78D, V147D, H235D, 1263V, K325N, V540C; R53T, P65D, L71D, N79D, L93D, V147D, W183N, H235D, S336C; L71D, N78D, G117A, V147D, W183N, I263V, K325N, S336C, V540C; R53T, P65D, L71D, N78D, N79D, L93D, V147D, W183N, H235D, K325N, S336C, V540C; L71D, L93D, V147D, H235D, I263V; R53T, V147D, 1151L, W183N, H235D, S336C, V540C; R53T, N78D, N79D, G117A, V147D, S336C; R53T, P65D, L71D, N79D, L93D, V147D, W183N, H235D, S336C; R53T, L71D, N78D, G117A, V147D, H235D, S336C, V540C; R53T, P65D, N78D, G117A, V147D, H235D, K325N, S336C, V540C; R53T, P65D, N78D, L93D, V147D, W183N, H235D, V540C; R53T, N78D, V147D, W183N, H235D, 1263V, S336C; R53T, N79D, V147D, W183N, H235D, 1263V, K325N, S336C; R53T, P65D, L71D, N78D, V147D, H235D, 1263V, S336C, V540C; R53T, L71D, G117A, V147D, H235D, 1263V, V540C; R53T, L71D, N78D, G117A, V147D, H235D, 1263V, K325N, S336C, V540C; R53T, P65D, N78D, N79D, V147D, S336C, V540C: R53T, N78D, N79D, V147D, W183N, H235D, 1263V, K325N; R53T, 1151L, H235D, K325N, S336C; or R53T, P65D, L71D, N79D, L93D, V147D, W183N, H235D, S336C, when aligned with and in reference to SEQ ID NO: 137.

In another aspect the invention generally provides for a genetically modified host cell containing an enzyme having at least 80% sequence identity to the amino acid sequence of any of the enzymes having CBDaS activity or to the amino acid sequence of a CBDaS or a portion thereof provided herein.

In an embodiment the host cell is a yeast cell or a yeast strain. In a preferred embodiment the yeast cell or the yeast strain is Saccharomyces cerevisiae.

In another aspect the invention provides for a method for producing CBDa or CBD, involving: culturing the genetically modified host cell of the invention in a medium with a carbon source under conditions suitable for making CBDa or CBD; and recovering CBDa or

CBD from the genetically modified host cell or the medium.

In another aspect the invention provides for a fermentation composition containing CBDa or CBD, and also containing: the genetically modified host cell of the invention; and CBDa or CBD produced by the genetically modified host cell. In an embodiment of the invention the CBDa or the CBD produced by the genetically modified host cell is within the genetically modified host cell.

In yet another aspect the invention provides for a non-naturally occurring enzyme having CBDaS activity, having an amino acid sequence that is at least 80% identical to the amino acid sequence of SEQ ID NO: 4, 7, 8, 9, 10, 11, 13, 15, 17, 19, 21, 134, 135, 136, 137, 147, 148, 149, 150, or 151. In an embodiment the non-naturally occurring enzyme having CBDaS activity contains one or more of the following mutations when aligned with and in reference to SEQ ID NO: 136: N45Q, N65Q, S168N, N296Q, N304Q, N328Q, N498Q, T47S, T67S, S170T, T298S, T306S, S330T, or T500S. In another embodiment the non-naturally occurring enzyme having CBDaS activity contains one or more amino acid substitutions occurring at position(s) 29, 31, 43, 49, 53, 56, 57, 65, 71, 78, 79, 93, 95, 103, 117, 125, 129, 143, 147, 151, 161, 183, 213, 235, 241, 263, 264, 285, 286, 303, 314, 325, 336, 339, 396, 436, 518, or 540 when aligned with and in reference to SEQ ID NO: 137. In further embodiments the one or more amino acid substitutions is: N29G, R31T, P43D, L49D, R53T, N56D, N57D, P65D, L71D, L71S, N78D, N79D, L93D, G95A, V103Y, G117A, V125D, 1129L, H143A, V147D, 1151L, W161R, W161A, W16IN, W161S, W16IT, W161D, W161H, W183N, H213D, H213N, H235D, I241V, I263V, E264P, D285N, K303N, S314C, K325N, S336C, T339S, F396L, A436G, V518C, or V540C. In yet another embodiment the non-naturally occurring enzyme having CBDaS activity contains one or more of the following sets of amino acid substitutions: R53T, N78D, V147D, H235D, 1263V, K325N, and V540C; R53T, P65D, L71D, N79D, L93D, V147D, W183N, H235D, and S336C; L71D, N78D, G117A, V147D, W183N, 1263V, K325N, S336C, and V540C; R53T, P65D, L71D, N78D, N79D, L93D, V147D, W183N, H235D, K325N, S336C, and V540C; L71D, L93D, V147D, H235D, and I263V; R53T, V147D, 1151L, W183N, H235D, S336C, and V540C; R53T, N78D, N79D, G117A, V147D, and S336C; R53T, P65D, L71D, N79D, L93D, V147D, W183N, H235D, and S336C; R53T, L71D, N78D, G117A, V147D, H235D, S336C, and V540C; R53T, P65D, N78D, G117A, V147D, H235D, K325N, S336C, and V540C; R53T, P65D, N78D, L93D, V147D, W183N, H235D, and V540C; R53T, N78D, V147D, W183N, H235D, I263V, and S336C; R53T, N79D, V147D, W183N, H235D, 1263V, K325N, and S336C; R53T, P65D, L71D, N78D, V147D, H235D, 1263V, S336C, and V540C; R53T, L71D, G117A, V147D, H235D, 1263V, and V540C; R53T, L71D, N78D, G117A, V147D, H235D, I263V, K325N, S336C, and V540C; R53T, P65D, N78D, N79D, V147D, S336C, and V540C: R53T, N78D, N79D, V147D, W183N, H235D, 1263V, and K325N; R53T, 1151L, H235D, K325N, and S336C; or R53T, P65D, L71D, N79D, L93D, V147D, W183N, H235D, and S336C, when aligned with and in reference to SEQ ID NO: 137.

In an embodiment the non-naturally occurring enzyme having CBDaS activity has an amino acid sequence that is at least 80% identical to the amino acid sequence of any of the non-naturally occurring enzymes having CBDaS activity of the invention.

In another aspect of the invention the non-naturally occurring enzyme having CBDaS activity is a fusion protein. In an embodiment the fusion protein comprises an amino acid sequence of a CBDaS or a portion thereof. In another embodiment the fusion protein has an amino acid sequence that is at least 80% identical to the amino acid sequence of SEQ ID NO: 1, 4, 7, 8, 9, 10, 11, 13, 15, 17, 19, 21, 134, 135, 136, 137, 147, 148, 149, 150, or 151. In yet another embodiment the fusion protein contains an amino acid sequence of a carrier protein or a portion thereof. In yet another embodiment the fusion protein has an amino acid sequence that is at least 80% identical to the amino acid sequence of SEQ ID NO: 34, 36, 73, 77, 81, 87, 89, 90, 91, 103, 104, 105, 106, 107, 108, 109, 110, 111, or 112. In an embodiment the fusion protein has an amino acid sequence of a signal sequence or a portion thereof. In another embodiment the fusion protein has an amino acid sequence at least 80% identical to the amino acid sequence of SEQ ID NOS: 42, 43, 44, 45, 46, 47, 48, 49, 50, 51, 52, 53, or 54. In further embodiments the fusion protein comprises an amino acid sequence of a linker or a portion thereof. In other embodiments the fusion protein has an amino acid sequence that is at least 80% identical to the amino acid sequence of SEQ ID NOS: 113, 114, 115, 116, 117, 118, 119, 120, 121, 122, 123, 124, 125, 152, or 172. In further embodiments the fusion protein has an amino acid sequence of a protease recognition site. In an embodiment the protease recognition site contains an amino acid sequence of RR, KR, RRK, RRQ, RRW, RRE, LDKR, LDKREAEA, or KREAEA. In an embodiment the fusion protein has an amino acid sequence of a mating factor alpha (MFα) or a portion thereof. In another embodiment the fusion protein has an amino acid sequence that is at least 80% identical to the amino acid sequence of SEQ ID NOS: 153, 154, 155, 156, or 157.

In a preferred embodiment the fusion protein contains two or more of: an amino acid sequence of a CBDaS or a portion thereof; an amino acid sequence that is at least 80% identical to the amino acid sequence of SEQ ID NOS: 1, 4, 7, 8, 9, 10, 11, 13, 15, 17, 19, 21, 4, 7, 8, 9, 10, 11, 13, 15, 17, 19, 21, 134, 135, 136, 137, 147, 148, 149, 150, or 151; an amino acid sequence of a carrier protein or a portion thereof; an amino acid sequence that is at least 80% identical to the amino acid sequence of SEQ ID NOS: 34, 36, 73, 77, 81, 87, 89, 90, 91, 103, 104, 105, 106, 107, 108, 109, 110, 111, or 112: an amino acid sequence of a signal sequence or a portion thereof; an amino acid sequence that is at least 80% identical to the amino acid sequence of SEQ ID NOS: 42, 43, 44, 45, 46, 47, 48, 49, 50, 51, 52, 53, or 54; an amino acid sequence of a linker or a portion thereof; an amino acid sequence at least 80% identical to the amino acid sequence of SEQ ID NOS: 113, 114, 115, 116, 117, 118, 119, 120, 121, 122, 123, 124, 125, 152, or 172; an amino acid sequence of a protease recognition site; a protease recognition site containing the amino acid sequence of RR, KR, RRK, RRQ, RRW, RRE, LDKR, LDKREAEA, or KREAEA; an amino acid sequence of a mating factor alpha (MFα) or a portion thereof; or an amino acid sequence that is at least 80% identical to the amino acid sequence of SEQ ID NOS: 153, 154, 155, 156, or 157.

In an embodiment the non-naturally occurring enzyme having CBDaS activity or the amino acid sequence of a CBDaS or a portion thereof contains one or more of the following mutations when aligned with and in reference to SEQ ID NO: 136: N45Q, N65Q, S168N, N296Q, N304Q, N328Q, N498Q, T47S, T67S, S170T, T298S, T306S, S330T, or T500S. In another embodiment the non-naturally occurring enzyme having CBDaS activity or the amino acid sequence of a CBDaS or a portion thereof has one or more amino acid substitutions occurring at position(s) 29, 31, 43, 49, 53, 56, 57, 65, 71, 78, 79, 93, 95, 103, 117, 125, 129, 143, 147, 151, 161, 183, 213, 235, 241, 263, 264, 285, 286, 303, 314, 325, 336, 339, 396, 436, 518, or 540 when aligned with and in reference to SEQ ID NO: 137. In another embodiment the one or more amino acid substitutions is: N29G, R31T, P43D, L49D, R53T, N56D, N57D, P65D, L71D, L71S, N78D, N79D, L93D, G95A, V103Y, G117A, V125D, 1129L, H143A, V147D, 1151L, W161R, W161A, W16IN, W161S, W161T, W161D, W161H, W183N, H213D, H213N, H235D, 1241V, 1263V, E264P, D285N, K303N, S314C, K325N, S336C, T339S, F396L, A436G, V518C, or V540C. In yet another embodiment the non-naturally occurring enzyme having CBDaS activity or the amino acid sequence of a CBDaS or a portion thereof contains one or more of the following amino acid substitutions: R53T, N78D, V147D, H235D, 1263V, K325N, and V540C; R53T, P65D, L71D, N79D, L93D, V147D, W183N, H235D, and S336C; L71D, N78D, G117A, V147D, W183N, 1263V, K325N, S336C, and V540C; R53T, P65D, L71D, N78D, N79D, L93D, V147D, W183N, H235D, K325N, S336C, and V540C; L7ID, L93D, V147D, H235D, and I263V; R53T, V147D, 1151L, W183N, H235D, S336C, and V540C; R53T, N78D, N79D, G117A, V147D, and S336C; R53T, P65D, L71D, N79D, L93D, V147D, W183N, H235D, and S336C; R53T, L71D, N78D, G117A, V147D, H235D, S336C, and V540C; R53T, P65D, N78D, G117A, V147D, H235D, K325N, S336C, and V540C; R53T, P65D, N78D, L93D, V147D, W183N, H235D, and V540C; R53T, N78D, V147D, W183N, H235D, 1263V, and S336C; R53T, N79D, V147D, W183N, H235D, 1263V, K325N, and S336C; R53T, P65D, L71D, N78D, V147D, H235D, 1263V, S336C, and V540C; R53T, L71D, G117A, V147D, H235D, 1263V, and V540C; R53T, L71D, N78D, G117A, V147D, H235D, I263V, K325N, S336C, and V540C; R53T, P65D, N78D, N79D, V147D, S336C, and V540C; R53T, N78D, N79D, V147D, W183N, H235D, 1263V, and K325N; R53T, 1151L, H235D, K325N, and S336C; or R53T, P65D, L71D, N79D, L93D, V147D, W183N, H235D, and S336C, when aligned with and in reference to SEQ ID NO: 137.

In an embodiment of the invention the non-naturally occurring enzyme having CBDaS activity comprising an amino acid sequence that is at least 80% identical to the amino acid sequence of any of the non-naturally occurring enzymes having CBDaS activity or to the amino acid sequence of a CBDaS or portion thereof provided herein. In another aspect the invention provides for a non-naturally occurring nucleic acid encoding the non-naturally occurring enzyme having CBDaS activity provided herein.

### BRIEF DESCRIPTION OF THE DRAWINGS

FIG. 1 is a schematic of the cannabinoid biosynthetic pathway. CBDa is synthesized from CBGa by the CBDaS enzyme.

FIG. 2 is a schematic of a “landing pad” approach to introduce genes into a host cell. An intergenic region in a host cell strain can be altered to contain an F-CphI endonuclease recognition site, flanked by a strong, GAL-regulon promoter and a terminator, as described in, for example, U.S. Pat. No. 7,919,605. This site allowed candidate genes to be integrated into the host genome by co-transformation of the endonuclease alongside donor DNA containing the desired DNA sequence to be screened, flanked by 40 base pair homology regions to the promoter and terminator.

FIG. 3 is a graph showing relative CBDa titers obtained from twelve different fusion proteins comprising CBDaS having various N-terminal truncations (removing the native signal sequence) fused to the PEP4 signal sequence of Komagataella pastoris. The highest CBDaS activity was observed from Trunc. 8.

FIG. 4 is a graph showing relative CBDa titers obtained from nine CBDaS natural diversity variants, identified using the reference CBDaS of SEQ ID NO: 1 as the basis for a BLAST query for UniParc. All variants were screened for CBDaS activity using the same 41-28aa truncation as Trunc. 8 (see FIG. 3 and Example 5) fused to the PEP4 signal sequence of Komagataella pastoris. The highest CBDaS activity was observed from Diversity Variant 6 (SEQ ID NO: 19), which showed about 3-fold higher activity than Trunc. 8.

FIG. 5 is a schematic of yeast surface display constructs used to fuse carrier proteins to CBDaS.

FIG. 6 is a graph showing relative CBDa titers obtained from a surface display carrier screen. CBDaS was fused to an array of carrier proteins, either at the carrier protein's N-terminus or C-terminus. Two native yeast carrier proteins, SAG1 (Carrier ID 17) and FLO5 (Carrier ID 11), showed CBDaS activity when the reference CBDaS (SEQ ID NO: 1) was fused to the carrier protein's N-terminus.

FIG. 7 is a graph showing relative CBDa titers obtained from a surface display signal sequence screen. Alternative yeast signal sequences were tested in place of the native AGA2 signal sequence (Sig. seq. 3) in a SAG1 surface display construct. Sig. seq. 2 and Sig. seqs. 4-14 showed CBDaS activity.

FIG. 8 is a graph showing relative CBDa titers obtained from surface display carrier protein truncation constructs. Various truncations of the carrier proteins SAG1 and FLO5 were tested, with multiple truncations of both SAG1 and FLO5 showing improved activity.

FIG. 9 is a graph showing relative CBDa titers obtained from a linker screen. Various linkers connecting the reference CBDaS (SEQ ID NO: 1) and a carrier protein (either SAG1 or FLO5) were tested. All linkers tested showed CBDaS activity except for a no-linker control.

FIG. 10 is a graph showing relative CBDa titers obtained from a KEX2 protease recognition site screen. KEX2 protease recognition sites were introduced between a signal sequence and the N-terminus of a CBDaS in various surface display expression constructs to force removal of the signal sequence. Multiple variants of the KEX2 recognition sequence were tested. In most cases, addition of KEX2 recognition sites showed improved CBDaS activity compared to constructs without a KEX2 recognition site.

FIG. 11 shows a graph of relative CBDa titers obtained from a screen of top SAG1 and FLO5 surface display constructs with different combinations of linkers, signal sequences, and carrier proteins.

FIG. 12 shows a graph of relative CBDa titers obtained from a screen of secretion constructs and vacuolar localization constructs, designed to target CBDaS secretion into the media or localize CBDaS to the vacuole. Multiple constructs showed improved CBDaS activity relative to Construct 178.

FIG. 13 shows a graph of relative CBDa titers obtained from a screen of CBDaS glycosylation site combinatorial mutants. Seven predicted CBDaS glycosylation sites were combinatorially mutagenized in five different constructs shown, to either eliminate glycosylation or alter the degree of glycosylation. Some constructs showed improved CBDaS activity compared to Construct 17.

FIG. 14 shows a graph of relative CBDa titers obtained from a screen of individual CBDaS point mutations. Site saturation mutagenesis was performed to mutate each position in a CBDaS (SEQ ID NO: 137) from a surface display construct (Construct 244). Multiple variants showed improved CBDaS activity, up to about 1.75 fold higher than Construct 244.

FIG. 15 shows a graph of relative CBDa titers obtained from a screen of CBDaS combinatorial mutants. The top individual CBDaS point mutants from Example 10 were consolidated together using a full factorial combinatorial library to produce variants with far higher activity than any single CBDaS point mutant. Mutations were introduced into SEQ ID NO: 137 using PCR, and variants were expressed in a top surface display expression construct (Construct 244). The majority of point mutant combinations led to improved CBDaS activity compared to Construct 244, with quite a few variants showing over 4-fold greater activity.

### DETAILED DESCRIPTION OF THE INVENTION

### Definitions

As used herein the singular forms “a,” “an,” and, “the” include plural reference unless the context clearly dictates otherwise.

The term “about” when modifying a numerical value or range herein includes normal variation encountered in the field, and includes plus or minus 1-10% (e.g., 1%, 2%, 3%, 4%, 5%, 6%, 7%, 8%, 9% or 10%) of the numerical value or end points of the numerical range. Thus, a value of 10 includes all numerical values from 9 to 11. All numerical ranges described herein include the endpoints of the range unless otherwise noted, and all numerical values in-between the end points, to the first significant digit.

As used herein, the term “cannabinoid” refers to a chemical substance that binds or interacts with a cannabinoid receptor (for example, a human cannabinoid receptor) and includes, without limitation, chemical compounds such endocannabinoids, phytocannabinoids, and synthetic cannabinoids. Synthetic compounds are chemicals made to mimic phytocannabinoids which are naturally found in the cannabis plant (e.g., Cannabis sativa), including but not limited to cannabigerols (CBG), cannabichromene (CBC), cannabidiol (CBD), tetrahydrocannabinol (THC), cannabinol (CBN), cannabinodiol (CBDL), cannabicyclol (CBL), cannabielsoin (CBE), and cannabitriol (CBT).

As used herein, the term “capable of producing” refers to a host cell which is genetically modified to include the enzymes necessary for the production of a given compound in accordance with a biochemical pathway that produces the compound. For example, a cell (e.g., a yeast cell) “capable of producing” a cannabinoid is one that contains the enzymes necessary for production of the cannabinoid according to the cannabinoid biosynthetic pathway.

As used herein, the term “exogenous” refers to a substance or compound that originated outside an organism or cell. The exogenous substance or compound can retain its normal function or activity when introduced into an organism or host cell described herein.

As used herein, the term “fermentation composition” refers to a composition which contains genetically modified host cells and products or metabolites produced by the genetically modified host cells. An example of a fermentation composition is a whole cell broth, which may be the entire contents of a vessel, including cells, aqueous phase, and compounds produced from the genetically modified host cells.

As used herein, the term “gene” refers to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an IRNA, tRNA, gRNA, or micro RNA.

A “genetic pathway” or “biosynthetic pathway” as used herein refer to a set of at least two different coding sequences, where the coding sequences encode enzymes that catalyze different parts of a synthetic pathway to form a desired product (e.g., a cannabinoid). In a genetic pathway a first encoded enzyme uses a substrate to make a first product which in turn is used as a substrate for a second encoded enzyme to make a second product. In some embodiments, the genetic pathway includes 3 or more members (e.g., 3, 4, 5, 6, 7, 8, 9, etc.), wherein the product of one encoded enzyme is the substrate for the next enzyme in the synthetic pathway.

As used herein, the term “genetic switch” refers to one or more genetic elements that allow controlled expression of enzymes, e.g., enzymes that catalyze the reactions of cannabinoid biosynthesis pathways. For example, a genetic switch can include one or more promoters operably linked to one or more genes encoding a biosynthetic enzyme, or one or more promoters operably linked to a transcriptional regulator which regulates expression one or more biosynthetic enzymes.

As used herein, the term “genetically modified” denotes a host cell that contains a heterologous nucleotide sequence. The genetically modified host cells described herein typically do not exist in nature.

As used herein, the term “heterologous” refers to what is not normally found in nature. The term “heterologous compound” refers to the production of a compound by a cell that does not normally produce the compound, or to the production of a compound at a level not normally produced by the cell. For example, a cannabinoid can be a heterologous compound.

A “heterologous genetic pathway” or a “heterologous biosynthetic pathway” as used herein refer to a genetic pathway that does not normally or naturally exist in an organism or cell.

The term “host cell” as used in the context of this invention refers to a microorganism, such as yeast, and includes an individual cell or cell culture contains a heterologous vector or heterologous polynucleotide as described herein. Host cells include progeny of a single host cell, and the progeny may not necessarily be completely identical (in morphology or in total DNA complement) to the original parent cell due to natural, accidental, or deliberate mutation and/or change. A host cell includes cells into which a recombinant vector or a heterologous polynucleotide of the invention has been introduced, including by transformation, transfection, and the like.

As used herein, the term “medium” refers to culture medium and/or fermentation medium.

The terms “modified,” “recombinant” and “engineered,” when used to describe a host cell described herein, refer to host cells or organisms that do not exist in nature, or express compounds, nucleic acids or proteins at levels that are not expressed by naturally occurring cells or organisms.

As used herein, the phrase “operably linked” refers to a functional linkage between nucleic acid sequences such that the linked promoter and/or regulatory region functionally controls expression of the coding sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as CLUSTAL, BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100multiplied by(the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid.

The terms “polynucleotide” and “nucleic acid” are used interchangeably and refer to a single or double-stranded polymer of deoxyribonucleotide or ribonucleotide bases read from the 5′ to the 3′ end. A nucleic acid as used in the present invention will generally contain phosphodiester bonds, although in some cases, nucleic acid analogs may be used that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); positive backbones; non-ionic backbones, and non-ribose backbones. Nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus, the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo—and ribo-nucleotides, and combinations of bases, including uracil, adenine, thymine, cytosine, guanine, inosine, xanthine hypoxanthine, isocytosine, isoguanine, etc. Nucleic acid sequences are presented in the 5′ to 3′ direction unless otherwise specified.

As used herein, the terms “polypeptide,” “peptide,” and “protein” are used interchangeably to refer to a polymer of amino acid residues. The terms encompass amino acid chains of any length, including full-length proteins, wherein the amino acid residues are linked by covalent peptide bonds.

As used herein, the term “production” generally refers to an amount of compound produced by a genetically modified host cell provided herein. In some embodiments, production is expressed as a yield of the compound by the host cell. In other embodiments, production is expressed as a productivity of the host cell in producing the compound.

As used herein, the term “productivity” refers to production of a compound by a host cell, expressed as the amount of non-catabolic compound produced (by weight) per amount of fermentation broth in which the host cell is cultured (by volume) over time (per hour).

As used herein, the term “promoter” refers to a synthetic or naturally derived nucleic acid that is capable of activating, increasing or enhancing expression of a DNA coding sequence, or inactivating, decreasing, or inhibiting expression of a DNA coding sequence. A promoter may contain one or more specific transcriptional regulatory sequences to further enhance or repress expression and/or to alter the spatial expression and/or temporal expression of the coding sequence. A promoter may be positioned 5′ (upstream) of the coding sequence under its control. A promoter may also initiate transcription in the downstream (3′) direction, the upstream (5′) direction, or be designed to initiate transcription in both the downstream (3′) and upstream (5′) directions. The distance between the promoter and a coding sequence to be expressed may be approximately the same as the distance between that promoter and the native nucleic acid sequence it controls. As is known in the art, variation in this distance may be accommodated without loss of promoter function. The term also includes a regulated promoter, which generally allows transcription of the nucleic acid sequence while in a permissive environment (e.g., microaerobic fermentation conditions, or the presence of maltose), but ceases transcription of the nucleic acid sequence while in a non-permissive environment (e.g., aerobic fermentation conditions, or in the absence of maltose). Promoters used herein can be constitutive, inducible, or repressible.

The term “yield” refers to production of a compound by a host cell, expressed as the amount of compound produced per amount of carbon source consumed by the host cell, by weight.

### High Efficiency Production of CBDa

In some embodiments, the disclosure features a host cell capable of producing CBDa or CBD. In some embodiments, the host cell contains one or more heterologous nucleic acids that each, independently, encodes an enzyme having CBDaS activity. In some embodiments, the enzyme having CBDaS activity is a fusion protein.

In some embodiments, the fusion protein comprises an amino acid sequence of a CBDas or a portion thereof. In some embodiments, the amino acid sequence of a CBDaS or a portion thereof comprises an amino acid sequence that is at least 80% identical to the amino acid sequence of SEQ ID NOS: 1, 4, 7, 8, 9, 10, 11, 13, 15, 17, 19, 21, 134, 135, 136, 137, 147, 148, 149, 150, or 151. In some embodiments, the fusion protein comprises an amino acid sequence that is at least 85% identical to the amino acid sequence of SEQ ID NOS: 1, 4, 7, 8, 9, 10, 11, 13, 15, 17, 19, 21, 134, 135, 136, 137, 147, 148, 149, 150, or 151. In some embodiments, the fusion protein comprises an amino acid sequence that is at least 90% identical to the amino acid sequence of SEQ ID NOS: 1, 4, 7, 8, 9, 10, 11, 13, 15, 17, 19, 21, 134, 135, 136, 137, 147, 148, 149, 150, or 151. In some embodiments, the fusion protein comprises an amino acid sequence that is at least 95% identical to the amino acid sequence of SEQ ID NOS: 1, 4, 7, 8, 9, 10, 11, 13, 15, 17, 19, 21, 134, 135, 136, 137, 147, 148, 149, 150, or 151. In some embodiments, the fusion protein comprises an amino acid sequence that is 100% identical to the amino acid sequence of SEQ ID NOS: 1, 4, 7, 8, 9, 10, 11, 13, 15, 17, 19, 21, 134, 135, 136, 137, 147, 148, 149, 150, or 151.

In some embodiments, the fusion protein comprises an amino acid sequence of a carrier protein or a portion thereof. In some embodiments, the amino acid sequence of a carrier protein or a portion thereof is at least 80% identical to the amino acid sequence of SEQ ID NO: 34, 36, 73, 77, 81, 87, 89, 90, 91, 103, 104, 105, 106, 107, 108, 109, 110, 111, or 112. In some embodiments, the amino acid sequence of a carrier protein or a portion thereof is at least 85% identical to the amino acid sequence of SEQ ID NO: 34, 36, 73, 77, 81, 87, 89, 90, 91, 103, 104, 105, 106, 107, 108, 109, 110, 111, or 112. In some embodiments, the amino acid sequence of a carrier protein or a portion thereof is at least 90% identical to the amino acid sequence of SEQ ID NO: 34, 36, 73, 77, 81, 87, 89, 90, 91, 103, 104, 105, 106, 107, 108, 109, 110, 111, or 112. In some embodiments, the amino acid sequence of a carrier protein or a portion thereof is at least 95% identical to the amino acid sequence of SEQ ID NO: 34, 36, 73, 77, 81, 87, 89, 90, 91, 103, 104, 105, 106, 107, 108, 109, 110, 111, or 112. In some embodiments, the amino acid sequence of a carrier protein or a portion thereof is 100% identical to the amino acid sequence of SEQ ID NO: 34, 36, 73, 77, 81, 87, 89, 90, 91, 103, 104, 105, 106, 107, 108, 109, 110, 111, or 112.

In some embodiments, the fusion protein comprises an amino acid sequence of a signal sequence or a portion thereof. In some embodiments, the amino acid sequence of a signal sequence or a portion thereof is at least 80% identical to the amino acid sequence of SEQ ID NOS: 42, 43, 44, 45, 46, 47, 48, 49, 50, 51, 52, 53, or 54. In some embodiments, the amino acid sequence of a signal sequence or a portion thereof is at least 85% identical to the amino acid sequence of SEQ ID NOS: 42, 43, 44, 45, 46, 47, 48, 49, 50, 51, 52, 53, or 54. In some embodiments, the amino acid sequence of a signal sequence or a portion thereof is at least 90% identical to the amino acid sequence of SEQ ID NOS: 42, 43, 44, 45, 46, 47, 48, 49, 50, 51, 52, 53, or 54. In some embodiments, the amino acid sequence of a signal sequence or a portion thereof is at least 95% identical to the amino acid sequence of SEQ ID NOS: 42, 43, 44, 45, 46, 47, 48, 49, 50, 51, 52, 53, or 54. In some embodiments, the amino acid sequence of a signal sequence or a portion thereof is 100% identical to the amino acid sequence of SEQ ID NOS: 42, 43, 44, 45, 46, 47, 48, 49, 50, 51, 52, 53, or 54.

In some embodiments, the fusion protein comprises an amino acid sequence of a linker or a portion thereof. In some embodiments, the amino acid sequence of a linker or a portion thereof is at least 80% identical to the amino acid sequence of SEQ ID NOS: 113, 114, 115, 116, 117, 118, 119, 120, 121, 122, 123, 124, 125, 152, or 172. In some embodiments, the amino acid sequence of a linker or a portion thereof is at least 85% identical to the amino acid sequence of SEQ ID NOS: 113, 114, 115, 116, 117, 118, 119, 120, 121, 122, 123, 124, 125, 152, or 172. In some embodiments, the amino acid sequence of a linker or a portion thereof is at least 90% identical to the amino acid sequence of SEQ ID NOS: 113, 114, 115, 116, 117, 118, 119, 120, 121, 122, 123, 124, 125, 152, or 172. In some embodiments, the amino acid sequence of a linker or a portion thereof is at least 95% identical to the amino acid sequence of SEQ ID NOS: 113, 114, 115, 116, 117, 118, 119, 120, 121, 122, 123, 124, 125, 152, or 172. In some embodiments, the amino acid sequence of a linker or a portion thereof is 100% identical to the amino acid sequence of SEQ ID NOS: 113, 114, 115, 116, 117, 118, 119, 120, 121, 122, 123, 124, 125, 152, or 172.

In some embodiments, the fusion protein comprises an amino acid sequence of a linker and an amino acid sequence of a carrier protein or a portion thereof. In some embodiments, the amino acid sequence of a linker or a portion thereof is at least 80%, at least 85%, at least 90%, at least 95%, or is 100% identical to the amino acid sequence of SEQ ID NOS: 113, 114, 115, 116, 117, 118, 119, 120, 121, 122, 123, 124, 125, 152, or 172, and the amino acid sequence of a carrier protein or a portion thereof is at least 80%, at least 85%, at least 90%, at least 95%, or is 100% identical to the amino acid sequence of SEQ ID NOS: 34, 36, 73, 77, 81, 87, 89, 90, 91, 103, 104, 105, 106, 107, 108, 109, 110, 111, or 112.

In some embodiments, the fusion protein comprises an amino acid sequence of a protease recognition site. In some embodiments, the protease recognition site is selected from the group of amino acid sequences consisting of RR, KR, RRK, RRQ, RRW, RRE, LDKR, LDKREAEA, and KREAEA.

In some embodiments, the fusion protein comprises an amino acid sequence of a mating factor alpha (MFα) or a portion thereof. In some embodiments, the amino acid sequence of a MFα or a portion thereof is at least 80% identical to the amino acid sequence of SEQ ID NOS: 153, 154, or 155. In some embodiments, the amino acid sequence of a MFα or a portion thereof is at least 85% identical to the amino acid sequence of SEQ ID NOS: 153, 154, or 155. In some embodiments, the amino acid sequence of a MFα or a portion thereof is at least 90% identical to the amino acid sequence of SEQ ID NOS: 153, 154, or 155. In some embodiments, the amino acid sequence of a MFα or a portion thereof is at least 95% identical to the amino acid sequence of SEQ ID NOS: 153, 154, or 155. In some embodiments, the amino acid sequence of a MFα or a portion thereof is 100% identical to the amino acid sequence of SEQ ID NOS: 153, 154, or 155.

In some embodiments, the fusion protein comprises an amino acid sequence that is at least 80% identical to the amino acid sequence of SEQ ID NOS: 156, or 157. In some embodiments, the fusion protein comprises an amino acid sequence that is at least 85% identical to the amino acid sequence of SEQ ID NOS: 156, or 157. In some embodiments, the fusion protein comprises an amino acid sequence that is at least 90% identical to the amino acid sequence of SEQ ID NOS: 156, or 157. In some embodiments, the fusion protein comprises an amino acid sequence that is at least 95% identical to the amino acid sequence of SEQ ID NOS: 156, or 157. In some embodiments, the fusion protein comprises an amino acid sequence that is 100% identical to the amino acid sequence of SEQ ID NOS: 156, or 157.

In some embodiments, the fusion protein comprises two or more of (a) an amino acid sequence of a CBDaS or a portion thereof, (b) an amino acid sequence that is at least 80% identical to the amino acid sequence of SEQ ID NOS: 1, 4, 7, 8, 9, 10, 11, 13, 15, 17, 19, 21, 4, 7, 8, 9, 10, 11, 13, 15, 17, 19, 21, 134, 135, 136, 137, 147, 148, 149, 150, or 151, (c) an amino acid sequence of a carrier protein or a portion thereof, (d) an amino acid sequence that is at least 80% identical to the amino acid sequence of SEQ ID NOS: 34, 36, 73, 77, 81, 87, 89, 90, 91, 103, 104, 105, 106, 107, 108, 109, 110, 111, or 112, (e) an amino acid sequence of a signal sequence or a portion thereof, (f) an amino acid sequence that is at least 80% identical to the amino acid sequence of SEQ ID NOS: 42, 43, 44, 45, 46, 47, 48, 49, 50, 51, 52, 53, or 54, (g) an amino acid sequence of a linker or a portion thereof, (h) an amino acid sequence at least 80% identical to the amino acid sequence of SEQ ID NOS: 113, 114, 115, 116, 117, 118, 119, 120, 121, 122, 123, 124, 125, 152, or 172, (i) an amino acid sequence of a protease recognition site, (j) a protease recognition site selected from the group of amino acid sequences consisting of RR, KR, RRK, RRQ, RRW, RRE, LDKR, LDKREAEA, and KREAEA, (k) an amino acid sequence of a mating factor alpha (MFα) or a portion thereof, or (1) an amino acid sequence that is at least 80% identical to the amino acid sequence of SEQ ID NOS: 153, 154, 155, 156, or 157.

In some embodiments, the enzyme having CBDaS activity or the amino acid sequence of a CBDaS or a portion thereof comprises one or more of the following mutations when aligned with and in reference to SEQ ID NO: 136: N45Q, N65Q, S168N, N296Q, N304Q, N328Q, N498Q, T47S, T67S, S170T, T298S, T306S, S330T, or T500S.

In some embodiments, the enzyme having CBDaS activity or the amino acid sequence of a CBDaS or a portion thereof comprises one or more amino acid substitutions occurring at position(s) 29, 31, 43, 49, 53, 56, 57, 65, 71, 78, 79, 93, 95, 103, 117, 125, 129, 143, 147, 151, 161, 183, 213, 235, 241, 263, 264, 285, 286, 303, 314, 325, 336, 339, 396, 436, 518, or 540 when aligned with and in reference to SEQ ID NO: 137. In some embodiments, the one or more amino acid substitutions is: N29G, R31T, P43D, L49D, R53T, N56D, N57D, P65D, L71D, L71S, N78D, N79D, L93D, G95A, V103Y, G117A, V125D, 1129L, H143A, V147D, 1151L, W161R, W161A, W161N, W161S, W161T, W161D, W161H, W183N, H213D, H213N, H235D, I241V, I263V, E264P, D285N, K303N, S314C, K325N, S336C, T339S, F396L, A436G, V518C, and/or V540C, when aligned with and in reference to SEQ ID NO: 137.

In some embodiments, the enzyme having CBDaS activity or the amino acid sequence of a CBDaS or a portion thereof comprises one or more amino acid substitutions selected from the group consisting of:
a) R53T, N78D, V147D, H235D, 1263V, K325N, and V540C;
b) R53T, P65D, L71D, N79D, L93D, V147D, W183N, H235D, and S336C;
c) L71D, N78D, G117A, V147D, W183N, 1263V, K325N, S336C, and V540C;
d) R53T, P65D, L71D, N78D, N79D, L93D, V147D, W183N, H235D, K325N, S336C, and V540C;
e) L71D, L93D, V147D, H235D, and I263V;
f) R53T, V147D, 1151L, W183N, H235D, S336C, and V540C;
g) R53T, N78D, N79D, G117A, V147D, and S336C;
h) R53T, P65D, L71D, N79D, L93D, V147D, W183N, H235D, and S336C;
i) R53T, L71D, N78D, G117A, V147D, H235D, S336C, and V540C;
j) R53T, P65D, N78D, G117A, V147D, H235D, K325N, S336C, and V540C;
k) R53T, P65D, N78D, L93D, V147D, W183N, H235D, and V540C;
1) R53T, N78D, V147D, W183N, H235D, 1263V, and S336C;
m) R53T, N79D, V147D, W183N, H235D, I263V, K325N, and S336C;
n) R53T, P65D, L71D, N78D, V147D, H235D, 1263V, S336C, and V540C;
0) R53T, L71D, G117A, V147D, H235D, 1263V, and V540C;
p) R53T, L71D, N78D, G117A, V147D, H235D, 1263V, K325N, S336C, and V540C;
q) R53T, P65D, N78D, N79D, V147D, S336C, and V540C;
r) R53T, N78D, N79D, V147D, W183N, H235D, 1263V, and K325N;
s) R53T, 1151L, H235D, K325N, and S336C; and
t) R53T, P65D, L71D, N79D, L93D, V147D, W183N, H235D, and S336C, when aligned with and in reference to SEQ ID NO: 137.

In some embodiments, the genetically modified host cell comprises an enzyme having at least 80% sequence identity to the amino acid sequence of any of the preceding enzymes having CBDaS activity or to the amino acid sequence of a CBDaS or a portion thereof.

In some embodiments, the host cell is a yeast cell or a yeast strain. In some embodiments, the yeast cell or the yeast strain is Saccharomyces cerevisiae.

In some embodiments, the disclosure features a method for producing CBDa or CBD, comprising culturing a genetically modified host cell capable of producing CBDa or CBD in a medium with a carbon source under conditions suitable for making CBDa or CBD, and recovering CBDa or CBD from the genetically modified host cell or the medium.

In some embodiments, the disclosure features a fermentation composition comprising a genetically modified host cell capable of producing CBDa or CBD, and CBDa or CBD produced by the genetically modified host cell. In some embodiments, the CBDa or CBD produced by the genetically modified host cell is within the genetically modified host cell.

In some embodiments, the disclosure features a non-naturally occurring enzyme having CBDaS activity, comprising an amino acid sequence that is at least 80% identical to the amino acid sequence of SEQ ID NO: 4, 7, 8, 9, 10, 11, 13, 15, 17, 19, 21, 134, 135, 136, 137, 147, 148, 149, 150, or 151.

In some embodiments, the non-naturally occurring enzyme having CBDaS activity comprises one or more of the following mutations when aligned with and in reference to SEQ ID NO: 136: N45Q, N65Q, S168N, N296Q, N304Q, N328Q, N498Q, T47S, T67S, S170T, T298S, T306S, S330T, or T500S.

In some embodiments, the non-naturally occurring enzyme having CBDaS activity comprises one or more amino acid substitutions occurring at position(s) 29, 31, 43, 49, 53, 56, 57, 65, 71, 78, 79, 93, 95, 103, 117, 125, 129, 143, 147, 151, 161, 183, 213, 235, 241, 263, 264, 285, 286, 303, 314, 325, 336, 339, 396, 436, 518, or 540 when aligned with and in reference to SEQ ID NO: 137. In some embodiments, the one or more amino acid substitutions is selected from the group consisting of: N29G, R31T, P43D, L49D, R53T, N56D, N57D, P65D, L71D, L71S, N78D, N79D, L93D, G95A, V103Y, G117A, V125D, 1129L, H143A, V147D, 1151L, W161R, W161A, W16IN, W161S, W16IT, W161D, W161H, W183N, H213D, H213N, H235D, I241V, I263V, E264P, D285N, K303N, S314C, K325N, S336C, T339S, F396L, A436G, V518C, and V540C when aligned with and in reference to SEQ ID NO: 137.

In some embodiments, the non-naturally occurring enzyme having CBDaS activity comprises one or more amino acid substitutions selected from the group consisting of:
a) R53T, N78D, V147D, H235D, 1263V, K325N, and V540C;
b) R53T, P65D, L71D, N79D, L93D, V147D, W183N, H235D, and S336C;
c) L71D, N78D, G117A, V147D, W183N, I263V, K325N, S336C, and V540C;
d) R53T, P65D, L71D, N78D, N79D, L93D, V147D, W183N, H235D, K325N, S336C, and V540C;
e) L71D, L93D, V147D, H235D, and I263V;
f) R53T, V147D, 1151L, W183N, H235D, S336C, and V540C;
g) R53T, N78D, N79D, G117A, V147D, and S336C;
h) R53T, P65D, L71D, N79D, L93D, V147D, W183N, H235D, and S336C;
i) R53T, L71D, N78D, G117A, V147D, H235D, S336C, and V540C;
j) R53T, P65D, N78D, G117A, V147D, H235D, K325N, S336C, and V540C;
k) R53T, P65D, N78D, L93D, V147D, W183N, H235D, and V540C;
1) R53T, N78D, V147D, W183N, H235D, 1263V, and S336C;
m) R53T, N79D, V147D, W183N, H235D, I263V, K325N, and S336C;
n) R53T, P65D, L71D, N78D, V147D, H235D, I263V, S336C, and V540C;
0) R53T, L71D, G117A, V147D, H235D, I263V, and V540C;
p) R53T, L71D, N78D, G117A, V147D, H235D, I263V, K325N, S336C, and V540C;
q) R53T, P65D, N78D, N79D, V147D, S336C, and V540C;
I) R53T, N78D, N79D, V147D, W183N, H235D, I263V, and K325N;
S) R53T, 1151L, H235D, K325N, and S336C; and
t) R53T, P65D, L71D, N79D, L93D, V147D, W183N, H235D, and S336C, when aligned with and in reference to SEQ ID NO: 137.

In some embodiments, the non-naturally occurring enzyme comprises an amino acid sequence that is at least 80% identical to the amino acid sequence of any of the non-naturally occurring enzymes having CBDaS activity in the preceding paragraph.

In some embodiments, the non-naturally occurring enzyme having CBDaS activity is a fusion protein. In some embodiments, the fusion protein comprises an amino acid sequence of a CBDaS or a portion thereof. In some embodiments, the fusion protein comprises an amino acid sequence that is at least 80% identical to the amino acid sequence of SEQ ID NOS: 1, 4, 7, 8, 9, 10, 11, 13, 15, 17, 19, 21, 134, 135, 136, 137, 147, 148, 149, 150, or 151. In some embodiments, the fusion protein comprises an amino acid sequence that is at least 85% identical to the amino acid sequence of SEQ ID NOS: 1, 4, 7, 8, 9, 10, 11, 13, 15, 17, 19, 21, 134, 135, 136, 137, 147, 148, 149, 150, or 151. In some embodiments, the fusion protein comprises an amino acid sequence that is at least 90% identical to the amino acid sequence of SEQ ID NOS: 4, 7, 8, 9, 10, 11, 13, 15, 17, 19, 21, 134, 135, 136, 137, 147, 148, 149, 150, or 151. In some embodiments, the fusion protein comprises an amino acid sequence that is at least 95% identical to the amino acid sequence of SEQ ID NOS: 4, 7, 8, 9, 10, 11, 13, 15, 17, 19, 21, 134, 135, 136, 137, 147, 148, 149, 150, or 151. In some embodiments, the fusion protein comprises an amino acid sequence that is 100% identical to the amino acid sequence of SEQ ID NOS: 4, 7, 8, 9, 10, 11, 13, 15, 17, 19, 21, 134, 135, 136, 137, 147, 148, 149, 150, or 151.

In some embodiments, the fusion protein comprises an amino acid sequence of a carrier protein or a portion thereof. In some embodiments, the fusion protein comprises an amino acid sequence that is at least 80% identical to the amino acid sequence of SEQ ID NO: 34, 36, 73, 77, 81, 87, 89, 90, 91, 103, 104, 105, 106, 107, 108, 109, 110, 111, or 112. In some embodiments, the fusion protein comprises an amino acid sequence that is at least 85% identical to the amino acid sequence of SEQ ID NO: 34, 36, 73, 77, 81, 87, 89, 90, 91, 103, 104, 105, 106, 107, 108, 109, 110, 111, or 112. In some embodiments, the fusion protein comprises an amino acid sequence that is at least 90% identical to the amino acid sequence of SEQ ID NO: 34, 36, 73, 77, 81, 87, 89, 90, 91, 103, 104, 105, 106, 107, 108, 109, 110, 111, or 112. In some embodiments, the fusion protein comprises an amino acid sequence that is at least 95% identical to the amino acid sequence of SEQ ID NO: 34, 36, 73, 77, 81, 87, 89, 90, 91, 103, 104, 105, 106, 107, 108, 109, 110, 111, or 112. In some embodiments, the fusion protein comprises an amino acid sequence that is 100% identical to the amino acid sequence of SEQ ID NO: 34, 36, 73, 77, 81, 87, 89, 90, 91, 103, 104, 105, 106, 107, 108, 109, 110, 111, or 112.

In some embodiments, the fusion protein comprises an amino acid sequence of a signal sequence or a portion thereof. In some embodiments, the fusion protein comprises an amino acid sequence that is at least 80% identical to the amino acid sequence of SEQ ID NOS: 42, 43, 44, 45, 46, 47, 48, 49, 50, 51, 52, 53, or 54. In some embodiments, the fusion protein comprises an amino acid sequence that is at least 85% identical to the amino acid sequence of SEQ ID NOS: 42, 43, 44, 45, 46, 47, 48, 49, 50, 51, 52, 53, or 54. In some embodiments, the fusion protein comprises an amino acid sequence that is at least 90% identical to the amino acid sequence of SEQ ID NOS: 42, 43, 44, 45, 46, 47, 48, 49, 50, 51, 52, 53, or 54. In some embodiments, the fusion protein comprises an amino acid sequence that is at least 95% identical to the amino acid sequence of SEQ ID NOS: 42, 43, 44, 45, 46, 47, 48, 49, 50, 51, 52, 53, or 54. In some embodiments, the fusion protein comprises an amino acid sequence that is 100% identical to the amino acid sequence of SEQ ID NOS: 42, 43, 44, 45, 46, 47, 48, 49, 50, 51, 52, 53, or 54.

In some embodiments, the fusion protein comprises an amino acid sequence of a linker or a portion thereof. In some embodiments, the fusion protein comprises an amino acid sequence that is at least 80% identical to the amino acid sequence of SEQ ID NOS: 113, 114, 115, 116, 117, 118, 119, 120, 121, 122, 123, 124, 125, 152, or 172. In some embodiments, the fusion protein comprises an amino acid sequence that is at least 85% identical to the amino acid sequence of SEQ ID NOS: 113, 114, 115, 116, 117, 118, 119, 120, 121, 122, 123, 124, 125, 152, or 172. In some embodiments, the fusion protein comprises an amino acid sequence that is at least 90% identical to the amino acid sequence of SEQ ID NOS: 113, 114, 115, 116, 117, 118, 119, 120, 121, 122, 123, 124, 125, 152, or 172. In some embodiments, the fusion protein comprises an amino acid sequence that is at least 95% identical to the amino acid sequence of SEQ ID NOS: 113, 114, 115, 116, 117, 118, 119, 120, 121, 122, 123, 124, 125, 152, or 172. In some embodiments, the fusion protein comprises an amino acid sequence that is 100% identical to the amino acid sequence of SEQ ID NOS: 113, 114, 115, 116, 117, 118, 119, 120, 121, 122, 123, 124, 125, 152, or 172.

In some embodiments, the fusion protein comprises an amino acid sequence of a linker and an amino acid sequence of a carrier protein or a portion thereof. In some embodiments, the fusion protein comprises an amino acid sequence that is at least 80%, at least 85%, at least 90%, at least 95%, or is 100% identical to the amino acid sequence of SEQ ID NOS: 113, 114, 115, 116, 117, 118, 119, 120, 121, 122, 123, 124, 125, 152, or 172, and an amino acid sequence that is at least 80%, at least 85%, at least 90%, at least 95%, or is 100% identical to the amino acid sequence of SEQ ID NOS: 34, 36, 73, 77, 81, 87, 89, 90, 91, 103, 104, 105, 106, 107, 108, 109, 110, 111, or 112.

In some embodiments, the fusion protein comprises an amino acid sequence of a protease recognition site. In some embodiments, the protease recognition site is selected from the group of amino acid sequences consisting of RR, KR, RRK, RRQ, RRW, RRE, LDKR, LDKREAEA, and KREAEA.

In some embodiments, the fusion protein comprises an amino acid sequence of a mating factor alpha (MFα) or a portion thereof. In some embodiments, the fusion protein comprises an amino acid sequence that is at least 80% identical to the amino acid sequence of SEQ ID NOS: 153, 154, or 155. In some embodiments, the fusion protein comprises an amino acid sequence that is at least 85% identical to the amino acid sequence of SEQ ID NOS: 153, 154, or 155. In some embodiments, the fusion protein comprises an amino acid sequence that is at least 90% identical to the amino acid sequence of SEQ ID NOS: 153, 154, or 155. In some embodiments, the fusion protein comprises an amino acid sequence that is at least 95% identical to the amino acid sequence of SEQ ID NOS: 153, 154, or 155. In some embodiments, the fusion protein comprises an amino acid sequence that is 100% identical to the amino acid sequence of SEQ ID NOS: 153, 154, or 155.

In some embodiments, the fusion protein comprises an amino acid sequence that is at least 80% identical to the amino acid sequence of SEQ ID NOS: 156, or 157. In some embodiments, the fusion protein comprises an amino acid sequence that is at least 85% identical to the amino acid sequence of SEQ ID NOS: 156, or 157. In some embodiments, the fusion protein comprises an amino acid sequence that is at least 90% identical to the amino acid sequence of SEQ ID NOS: 156, or 157. In some embodiments, the fusion protein comprises an amino acid sequence that is at least 95% identical to the amino acid sequence of SEQ ID NOS: 156, or 157. In some embodiments, the fusion protein comprises an amino acid sequence that is 100% identical to the amino acid sequence of SEQ ID NOS: 156, or 157.

In some embodiments, the fusion protein comprises two or more of (a) an amino acid sequence of a CBDaS or a portion thereof, (b) an amino acid sequence that is at least 80% identical to the amino acid sequence of SEQ ID NOS: 1, 4, 7, 8, 9, 10, 11, 13, 15, 17, 19, 21, 4, 7, 8, 9, 10, 11, 13, 15, 17, 19, 21, 134, 135, 136, 137, 147, 148, 149, 150, or 151, (c) an amino acid sequence of a carrier protein or a portion thereof, (d) an amino acid sequence that is at least 80% identical to the amino acid sequence of SEQ ID NOS: 34, 36, 73, 77, 81, 87, 89, 90, 91, 103, 104, 105, 106, 107, 108, 109, 110, 111, or 112, (e) an amino acid sequence of a signal sequence or a portion thereof, (f) an amino acid sequence that is at least 80% identical to the amino acid sequence of SEQ ID NOS: 42, 43, 44, 45, 46, 47, 48, 49, 50, 51, 52, 53, or 54, (g) an amino acid sequence of a linker or a portion thereof, (h) an amino acid sequence at least 80% identical to the amino acid sequence of SEQ ID NOS: 113, 114, 115, 116, 117, 118, 119, 120, 121, 122, 123, 124, 125, 152, or 172, (i) an amino acid sequence of a protease recognition site, (j) a protease recognition site selected from the group of amino acid sequences consisting of RR, KR, RRK, RRQ, RRW, RRE, LDKR, LDKREAEA, and KREAEA, (k) an amino acid sequence of a mating factor alpha (MFα) or a portion thereof, or (1) an amino acid sequence that is at least 80% identical to the amino acid sequence of SEQ ID NOS: 153, 154, 155, 156, or 157.

In some embodiments, the non-naturally occurring enzyme having CBDaS activity or the amino acid sequence of a CBDaS or a portion thereof comprises one or more of the following mutations when aligned with and in reference to SEQ ID NO: 136: N45Q, N65Q, S168N, N296Q, N304Q, N328Q, N498Q, T47S, T67S, S170T, T298S, T306S, S330T, or T500S.

In some embodiments, the non-naturally occurring enzyme having CBDaS activity or the amino acid sequence of a CBDaS or a portion thereof comprises one or more amino acid substitutions occurring at position(s) 29, 31, 43, 49, 53, 56, 57, 65, 71, 78, 79, 93, 95, 103, 117, 125, 129, 143, 147, 151, 161, 183, 213, 235, 241, 263, 264, 285, 286, 303, 314, 325, 336, 339, 396, 436, 518, or 540 when aligned with and in reference to SEQ ID NO: 137. In some embodiments, the one or more amino acid substitutions is selected from the group consisting of: N29G, R31T, P43D, L49D, R53T, N56D, N57D, P65D, L71D, L71S, N78D, N79D, L93D, G95A, V103Y, G117A, V125D, I129L, H143A, V147D, 1151L, W161R, W161A, W161N, W161S, W16IT, W161D, W161H, W183N, H213D, H213N, H235D, 1241V, 1263V, E264P, D285N, K303N, S314C, K325N, S336C, T339S, F396L, A436G, V518C, and V540C.

In some embodiments, the non-naturally occurring enzyme having CBDaS activity or the amino acid sequence of a CBDaS or a portion thereof comprises one or more amino acid substitutions selected from the group consisting of:
a) R53T, N78D, V147D, H235D, I263V, K325N, and V540C;
b) R53T, P65D, L71D, N79D, L93D, V147D, W183N, H235D, and S336C;
c) L71D, N78D, G117A, V147D, W183N, I263V, K325N, S336C, and V540C;
d) R53T, P65D, L71D, N78D, N79D, L93D, V147D, W183N, H235D, K325N, S336C, and V540C;
e) L71D, L93D, V147D, H235D, and I263V;
f) R53T, V147D, I151L, W183N, H235D, S336C, and V540C;
g) R53T, N78D, N79D, G117A, V147D, and S336C;
h) R53T, P65D, L71D, N79D, L93D, V147D, W183N, H235D, and S336C;
i) R53T, L71D, N78D, G117A, V147D, H235D, S336C, and V540C;
j) R53T, P65D, N78D, G117A, V147D, H235D, K325N, S336C, and V540C;
k) R53T, P65D, N78D, L93D, V147D, W183N, H235D, and V540C;
1) R53T, N78D, V147D, W183N, H235D, 1263V, and S336C;
m) R53T, N79D, V147D, W183N, H235D, I263V, K325N, and S336C;
n) R53T, P65D, L71D, N78D, V147D, H235D, 1263V, S336C, and V540C;
O) R53T, L71D, G117A, V147D, H235D, I263V, and V540C;
p) R53T, L71D, N78D, G117A, V147D, H235D, 1263V, K325N, S336C, and V540C;
q) R53T, P65D, N78D, N79D, V147D, S336C, and V540C;
r) R53T, N78D, N79D, V147D, W183N, H235D, I263V, and K325N;
S) R53T, I151L, H235D, K325N, and S336C; and
t) R53T, P65D, L71D, N79D, L93D, V147D, W183N, H235D, and S336C, when aligned with and in reference to SEQ ID NO: 137.

In some embodiments, the non-naturally occurring enzyme comprises an amino acid sequence that is at least 80% identical to the amino acid sequence of any of the non-naturally occurring enzymes having CBDaS activity or to the amino acid sequence of a CBDaS or a portion thereof in the preceding paragraph.

In some embodiments, the disclosure features a non-naturally occurring nucleic acid encoding the non-naturally occurring enzyme having CBDaS activity of the preceding paragraphs.

### Cannabinoid Biosynthetic Pathway

In an aspect, a host cell described herein includes one or more nucleic acids encoding one or more enzymes of a heterologous genetic pathway that produces a cannabinoid or a precursor of a cannabinoid. The cannabinoid biosynthetic pathway may begin with hexanoic acid as the substrate for an acyl activating enzyme (AAE) to produce hexanoyl-CoA, which is used by a tetraketide synthase (TKS) to produce tetraketide-CoA, which is used by an olivetolic acid cyclase (OAC) to produce olivetolic acid, which is used by a geranyl pyrophosphate (GPP) synthase and a cannabigerolic acid synthase (CBGaS) to produce a cannabigerolic acid (CBGa), which is used by a cannabidiolic acid synthase (CBDaS) to produce a cannabidiolic acid (CBDa). In some embodiments, CBGa or CBDa spontaneously decarboxylate, including upon heating, to form CBG and CBD, respectively. In some embodiments, the cannabinoid precursor that is produced is a substrate in the cannabinoid pathway (e.g., hexanoate or olivetolic acid). In some embodiments, the precursor is a substrate for an AAE, a TKS, an OAC, a CBGaS, a GPP synthase, a CBGaS, or a CBDaS. In some embodiments, the precursor, substrate, or intermediate in the cannabinoid pathway is hexanoate, olivetol, olivetolic acid, or CBGa. In some embodiments, the host cell does not contain the precursor, substrate or intermediate in an amount sufficient to produce the cannabinoid or a precursor of the cannabinoid. In some embodiments, the host cell does not contain hexanoate at a level or in an amount sufficient to produce the cannabinoid in an amount over 10 mg/L. In some embodiments, the heterologous genetic pathway encodes at least one enzyme selected from the group consisting of an AAE, a TKS, an OAC, a GPP synthase, a CBGaS, and a CBDaS. In some embodiments, the genetically modified host cell includes an AAE, TKS, OAC, a GPP synthase, a CBGaS, and a CBDaS.

The cannabinoid pathway, including the enzymes discussed in the following paragraphs, is described in U.S. Pat. No. 10,563,211, the disclosure of which is incorporated herein by reference.

In some embodiments, a host cell includes a heterologous acyl activating enzyme (AAE) such that the host cell is capable of producing a cannabinoid. The AAE may be from Cannabis sativa or may be an enzyme from another plant or fungal source which has been shown to have AAE activity in the cannabinoid biosynthetic pathway, resulting in the production of the cannabinoid precursor hexanoyl-CoA.

In some embodiments, a host cell includes a heterologous tetraketide synthase (TKS) such that the host cell is capable of producing a cannabinoid. A TKS uses the hexanoyl-CoA precursor to generate tetraketide-CoA. The TKS may be from Cannabis sativa or may be an enzyme from another plant, fungal, or bacterial source which has been shown to have TKS activity in the cannabinoid biosynthetic pathway, resulting in the production of the cannabinoid precursor tetraketide-CoA.

In some embodiments, a host cell includes a heterologous cannabigerolic acid synthase (CBGaS) such that the host cell is capable of producing a cannabinoid. A CBGaS uses the olivetolic acid precursor and geranyl pyrophosphate (GPP) precursor to generate cannabigerolic acid (CBGa). The CBGaS may be from Cannabis sativa or may be an enzyme from another plant, fungal, or bacterial source which has been shown to have CBGaS activity in the cannabinoid biosynthetic pathway, resulting in the production of the cannabinoid CBGa.

In some embodiments, a host cell includes a heterologous GPP synthase such that the host cell is capable of producing a cannabinoid. A GPP synthase uses the product of the isoprenoid biosynthesis pathway precursor to generate CBGa together with a prenyltransferase enzyme. The GPP synthase may be from Cannabis sativa or may be an enzyme from another plant, fungal, or bacterial source which has been shown to have GPP synthase activity in the cannabinoid biosynthetic pathway, resulting in the production of the cannabinoid CBGa.

In some embodiments, a host cell includes a heterologous CBDaS such that the host cell is capable of producing a cannabinoid. A CBDaS uses the CBGa precursor to generate CBDa. The CBDaS may be from Cannabis sativa or may be an enzyme from another plant, fungal, or bacterial source which has been shown to have CBDaS activity in the cannabinoid biosynthetic pathway, resulting in the production of the cannabinoid CBDa.

The host cell may further express other heterologous enzymes in addition to AAE, TKS, GPP synthase, CBGaS, and/or CBDaS. For example, in some embodiments, a host cell includes a heterologous olivetolic acid cyclase (OAC) such that the host cell is capable of producing a cannabinoid. An OAC uses the tetraketide-CoA precursor to generate olivetolic acid. The OAC may be from Cannabis sativa or may be an enzyme from another plant or fungal source which has been shown to have OAC activity in the cannabinoid biosynthetic pathway, resulting in the production of the cannabinoid precursor olivetolic acid. In some embodiments, the host cell may include a heterologous nucleic acid that encodes at least one enzyme from the mevalonate biosynthetic pathway. Enzymes which make up the mevalonate biosynthetic pathway may include but are not limited to an acetyl-CoA thiolase, a HMG-COA synthase, a HMG-COA reductase, a mevalonate kinase, a phosphomevalonate kinase, a mevalonate pyrophosphate decarboxylase, and an IPP: DMAPP isomerase. In some embodiments, the host cell includes a heterologous nucleic acid that encodes the acetyl-CoA thiolase, the HMG-COA synthase, the HMG-COA reductase, the mevalonate kinase, the phosphomevalonate kinase, the mevalonate pyrophosphate decarboxylase, and the IPP: DMAPP isomerase of the mevalonate biosynthesis pathway.

In some embodiments, the host cell may express heterologous enzymes of the central carbon metabolism. Enzymes of the central carbon metabolism may include an acetyl-CoA synthase, an aldehyde dehydrogenase, and a pyruvate decarboxylase. In some embodiments, the host cell includes heterologous nucleic acids that independently encode an acetyl-CoA synthase, and/or an aldehyde dehydrogenase, and/or a pyruvate decarboxylase. In some embodiments, the acetyl-CoA synthase and the aldehyde dehydrogenase from Saccharomyces cerevisiae, and the pyruvate decarboxylase from Zymomonas mobilis.

Due to the inherent degeneracy of the genetic code, other polynucleotides which encode substantially the same or functionally equivalent polypeptides can also be used to clone and express the polynucleotides encoding the protein components of the heterologous genetic pathway described herein.

As will be understood by those of skill in the art, it can be advantageous to modify a coding sequence to enhance its expression in a particular host. The genetic code is redundant with 64 possible codons, but most organisms typically use a subset of these codons more frequently. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.”

Optimized coding sequences containing codons preferred by a particular prokaryotic or eukaryotic host (Murray et al., 1989, Nucl Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., 1996, Nucl Acids Res. 24:216-8).

Those of skill in the art will recognize that, due to the degenerate nature of the genetic code, a variety of DNA molecules differing in their nucleotide sequences can be used to encode a given enzyme of the disclosure. Any one of the polypeptide sequences disclosed herein may be encoded by DNA molecules of any sequence that encode the amino acid sequences of the polypeptides and proteins of the enzymes utilized in the methods of the disclosure. In a similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for the compositions and methods provided herein are encompassed by the disclosure. In some embodiments, two proteins (or a region of the proteins) can be considered homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (e.g., Pearson W. R., 1994, Methods in Mol Biol 25:365-89).

The following six groups each contain amino acids that are conservative substitutions for one another: 1) Serine(S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. A typical algorithm used for comparing a molecule sequence to a database containing a large number of sequences from different organisms is the computer algorithm BLAST. When searching a database containing sequences from a large number of different organisms, it is typical to compare amino acid sequences.

Furthermore, any of the genes encoding the foregoing enzymes (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in a host cell, for example, a yeast.

In addition, genes encoding these enzymes can be identified from other fungal and bacterial species and can be expressed in the host cell. A variety of organisms could serve as sources for these enzymes, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorphs, Candida spp., Trichosporon spp., Yamadazyma spp., including Y. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include, but are not limited to, Piromyces spp., Orpinomyces spp., or Neocallimastix spp. Sources of prokaryotic enzymes that are useful include, but are not limited to, Escherichia coli, Zymomonas mobilis, Staphylococcus aureus, Bacillus spp., Clostridium spp., Corynebacterium spp., Pseudomonas spp., Lactococcus spp., Enterobacter spp., and Salmonella spp.

Techniques known to those skilled in the art may be suitable to identify additional homologous genes and homologous enzymes. Generally, analogous genes and/or analogous enzymes can be identified by functional analysis and will have functional similarities. Techniques known to those skilled in the art may be suitable to identify analogous genes and analogous enzymes. For example, to identify homologous or analogous kinase genes, proteins, or enzymes, techniques may include, but are not limited to, cloning a gene by PCR using primers based on a published sequence of a kinase gene/enzyme or by degenerate PCR using degenerate primers designed to amplify a conserved region among kinase genes. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity (e.g. as described herein or in Kiritani, K., Branched-Chain Amino Acids Methods Enzymology, 1970), then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, JGI Phyzome v12.1, BLAST, NCBI RefSeq, UniProt KB, or MetaCYC Protein annotations in the UniProt Knowledgebase may also be used to identify enzymes which have a similar function in addition to the National Center for Biotechnology Information RefSeq database. The candidate gene or enzyme may be identified within the above-mentioned databases in accordance with the teachings herein.

### Modified Host Cells

In one aspect, provided herein are host cells comprising at least one enzyme of the cannabinoid biosynthetic pathway. In some embodiments, the cannabinoid biosynthetic pathway contains a genetic regulatory element, such as a nucleic acid sequence, that is regulated by an exogenous agent. In some embodiments, the exogenous agent acts to regulate expression of the heterologous genetic pathway. Thus, in some embodiments, the exogenous agent can be a regulator of gene expression.

In some embodiments, the exogenous agent can be used as a carbon source by the host cell. For example, the same exogenous agent can both regulate production of a cannabinoid and provide a carbon source for growth of the host cell. In some embodiments, the exogenous agent is galactose. In some embodiments, the exogenous agent is maltose.

In some embodiments, the genetic regulatory element is a nucleic acid sequence, such as a promoter.

In some embodiments, the genetic regulatory element is a galactose-responsive promoter. In some embodiments, galactose positively regulates expression of the cannabinoid biosynthetic pathway, thereby increasing production of the cannabinoid. In some embodiments, the galactose-responsive promoter is a GAL1 promoter. In some embodiments, the galactose-responsive promoter is a GAL10 promoter. In some embodiments, the galactose-responsive promoter is a GAL2, GAL3, or GAL7 promoter. In some embodiments, heterologous genetic pathway contains the galactose-responsive regulatory elements described in Westfall et al. (PNAS (2012) vol. 109: E111-118). In some embodiments, the host cell lacks the gall gene and is unable to metabolize galactose, but galactose can still induce galactose-regulated genes.

TABLE A
Exemplary GAL Promoter Sequences

| Promoter | Sequence |
|---|---|
| pGAL1 | SEQ ID NO: 158 |
| pGAL10 | SEQ ID NO: 159 |
| pGAL2 | SEQ ID NO: 160 |
| pGAL3 | SEQ ID NO: 161 |
| pGAL7 | SEQ ID NO: 162 |
| pGAL4 | SEQ ID NO: 163 |

In some embodiments, the galactose regulation system used to control expression of one or more enzymes of the cannabinoid biosynthetic pathway is re-configured such that it is no longer induced by the presence of galactose. Instead, the gene of interest will be expressed unless repressors, which may be maltose in some strains, are present in the medium.

In some embodiments, the genetic regulatory element is a maltose-responsive promoter. In some embodiments, maltose negatively regulates expression of the cannabinoid biosynthetic pathway, thereby decreasing production of the cannabinoid. In some embodiments, the maltose-responsive promoter is selected from the group consisting of pMAL1, pMAL2, pMAL11, pMAL12, pMAL31 and pMAL32. The maltose genetic regulatory element can be designed to both activate expression of some genes and repress expression of others, depending on whether maltose is present or absent in the medium. Maltose regulation of gene expression and maltose-responsive promoters are described in U.S. Pat. No. 10,563,229, which is hereby incorporated by reference. Genetic regulation of maltose metabolism is described in Novak et al., “Maltose Transport and Metabolism in S. cerevisiae,” Food Technol. Biotechnol. 42 (3) 213-218 (2004).

TABLE B
Exemplary MAL Promoter Sequences

| Promoter | Sequence |
|---|---|
| pMAL1 | SEQ ID NO: 164 |
| pMAL2 | SEQ ID NO: 165 |
| pMAL11 | SEQ ID NO: 166 |
| pMAL12 | SEQ ID NO: 167 |
| pMAL31 | SEQ ID NO: 168 |
| pMAL32 | SEQ ID NO: 169 |

In some embodiments, the heterologous genetic pathway is regulated by a combination of the maltose and galactose regulons.

In some embodiments, the recombinant host cell does not contain, or expresses a very low level of (for example, an undetectable amount), a precursor (e.g., hexanoate) required to make the cannabinoid. In some embodiments, the precursor (e.g., hexanoate) is a substrate of an enzyme in the cannabinoid biosynthetic pathway.

### Yeast Strains

In some embodiments, yeast strains useful in the present methods include yeasts that have been deposited with microorganism depositories (e.g. IFO, ATCC, etc.) and belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, chizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In some embodiments, the strain is Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorphs (now known as Pichia angusta). In some embodiments, the host microbe is a strain of the genus Candida, such as Candida lipolytica, Candida guilliermondii, Candida krusei, Candida pseudotropicalis, or Candida utilis.

In a particular embodiment, the strain is Saccharomyces cerevisiae. In some embodiments, the host is a strain of Saccharomyces cerevisiae selected from the group consisting of Baker's yeast, CEN.PK, CEN.PK2, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, and AL-1. In some embodiments, the strain of Saccharomyces cerevisiae is CEN.PK.

In some embodiments, the strain is a microbe that is suitable for industrial fermentation. In particular embodiments, the microbe is conditioned to subsist under high solvent concentration, high temperature, expanded substrate utilization, nutrient limitation, osmotic stress due to sugar and salts, acidity, sulfite and bacterial contamination, or combinations thereof, which are recognized stress conditions of the industrial fermentation environment.

### Methods of Making the Host Cells

In another aspect, provided are methods of making the modified host cells described herein. In some embodiments, the methods include transforming a host cell with the heterologous nucleic acid constructs described herein which encode the proteins expressed by a heterologous genetic pathway described herein. Methods for transforming host cells are described in “Laboratory Methods in Enzymology: DNA,” edited by Jon Lorsch, Volume 529, (2013); and U.S. Pat. No. 9,200,270 to Hsieh, Chung-Ming, et al., and references cited therein.

### Methods for Producing a Cannabinoid

In another aspect, methods are provided for producing a cannabinoid are described herein. In some embodiments, the method decreases expression of the cannabinoid. In some embodiments, the method includes culturing a host cell comprising at least one enzyme of the cannabinoid biosynthetic pathway described herein in a medium comprising an exogenous agent, wherein the exogenous agent decreases the expression of the cannabinoid. In some embodiments, the exogenous agent is maltose. In some embodiments, the exogenous agent is maltose. In some embodiments, the method results in less than 0.001 mg/L of cannabinoid or a precursor thereof.

In some embodiments, the method is for decreasing expression of a cannabinoid or precursor thereof. In some embodiments, the method includes culturing a host cell comprising an AAE, and/or a TKS, and/or a CBGaS, and/or a GPP synthase, and/or CBDaS described herein in a medium comprising an exogenous agent, wherein the exogenous agent decreases the expression of the cannabinoid. In some embodiments, the exogenous agent is maltose. In some embodiments, the exogenous agent is maltose. In some embodiments, the method results in the production of less than 0.001 mg/L of a cannabinoid or a precursor thereof.

In some embodiments, the method increases the expression of a cannabinoid. In some embodiments, the method includes culturing a host cell comprising an AAE, and/or a TKS, and/or a CBGaS, and/or a GPP synthase, and/or CBDaS described herein in a medium comprising the exogenous agent, wherein the exogenous agent increases expression of the cannabinoid. In some embodiments, the exogenous agent is galactose. In some embodiments, the method further includes culturing the host cell with the precursor or substrate required to make the cannabinoid.

In some embodiments, the method increases the expression of a cannabinoid product or precursor thereof. In some embodiments, the method includes culturing a host cell comprising a heterologous cannabinoid pathway described herein in a medium comprising an exogenous agent, wherein the exogenous agent increases the expression of the cannabinoid or a precursor thereof. In some embodiments, the exogenous agent is galactose. In some embodiments, the method further includes culturing the host cell with a precursor or substrate required to make the cannabinoid or precursor thereof. In some embodiments, the precursor required to make the cannabinoid or precursor thereof is hexanoate. In some embodiments, the combination of the exogenous agent and the precursor or substrate required to make the cannabinoid or precursor thereof produces a higher yield of cannabinoid than the exogenous agent alone.

In some embodiments, the cannabinoid or a precursor thereof is cannabidiolic acid (CBDa), cannabidiol (CBD), cannabigerolic acid (CBGa), or cannabigerol (CBG).

### Culture and Fermentation Methods

Materials and methods for the maintenance and growth of microbial cultures are well known to those skilled in the art of microbiology or fermentation science (see, for example, Bailey et al., Biochemical Engineering Fundamentals, second edition, McGraw Hill, New York, 1986). Consideration must be given to appropriate culture medium, pH, temperature, and requirements for aerobic, microaerobic, or anaerobic conditions, depending on the specific requirements of the host cell, the fermentation, and the process.

The methods of producing cannabinoids provided herein may be performed in a suitable culture medium in a suitable container, including but not limited to a cell culture plate, a flask, or a fermentor. Further, the methods can be performed at any scale of fermentation known in the art to support industrial production of microbial products. Any suitable fermentor may be used including a stirred tank fermentor, an airlift fermentor, a bubble fermentor, or any combination thereof. In particular embodiments utilizing Saccharomyces cerevisiae as the host cell, strains can be grown in a fermentor as described in detail by Kosaric, et al, in Ullmann's Encyclopedia of Industrial Chemistry, Sixth Edition, Volume 12, pages 398-473, Wiley-VCH Verlag GmbH & Co. KDaA, Weinheim, Germany.

In some embodiments, the culture medium is any culture medium in which a genetically modified microorganism capable of producing a heterologous product can subsist, i.e., maintain growth and viability. In some embodiments, the culture medium is an aqueous medium comprising assimilable carbon, nitrogen and phosphate sources. Such a medium can also include appropriate salts, minerals, metals, and other nutrients. In some embodiments, the carbon source and each of the essential cell nutrients are added incrementally or continuously to the fermentation medium, and each required nutrient is maintained at essentially the minimum level needed for efficient assimilation by growing cells, for example, in accordance with a predetermined cell growth curve based on the metabolic or respiratory function of the cells which convert the carbon source to a biomass.

Suitable conditions and suitable medium for culturing microorganisms are well known in the art. In some embodiments, the suitable medium is supplemented with one or more additional agents, such as, for example, an inducer (e.g., when one or more nucleotide sequences encoding a gene product are under the control of an inducible promoter), a repressor (e.g., when one or more nucleotide sequences encoding a gene product are under the control of a repressible promoter), or a selection agent (e.g., an antibiotic to select for microorganisms comprising the genetic modifications).

In some embodiments, the carbon source is a monosaccharide (simple sugar), a disaccharide, a polysaccharide, a non-fermentable carbon source, or one or more combinations thereof. Non-limiting examples of suitable monosaccharides include glucose, galactose, mannose, fructose, ribose, and combinations thereof. Non-limiting examples of suitable disaccharides include sucrose, lactose, maltose, trehalose, cellobiose, and combinations thereof. Non-limiting examples of suitable polysaccharides include starch, glycogen, cellulose, chitin, and combinations thereof. Non-limiting examples of suitable non-fermentable carbon sources include acetate and glycerol.

The concentration of a carbon source, such as glucose or sucrose, in the culture medium should promote cell growth, but not be so high as to repress growth of the microorganism used. Typically, cultures are run with a carbon source, such as glucose or sucrose, being added at levels to achieve the desired level of growth and biomass. Production of cannabinoids may also occur in these culture conditions, but at undetectable levels (with detection limits being about <0.1 g/l). In other embodiments, the concentration of a carbon source, such as glucose or sucrose, in the culture medium is greater than about 1 g/L, preferably greater than about 2 g/L, and more preferably greater than about 5 g/L. In addition, the concentration of a carbon source, such as glucose or sucrose, in the culture medium is typically less than about 100 g/L, preferably less than about 50 g/L, and more preferably less than about 20 g/L. It should be noted that references to culture component concentrations can refer to both initial and/or ongoing component concentrations. In some cases, it may be desirable to allow the culture medium to become depleted of a carbon source during culture.

Sources of assimilable nitrogen that can be used in a suitable culture medium include, but are not limited to, simple nitrogen sources, organic nitrogen sources and complex nitrogen sources. Such nitrogen sources include anhydrous ammonia, ammonium salts and substances of animal, vegetable and/or microbial origin. Suitable nitrogen sources include, but are not limited to, protein hydrolysates, microbial biomass hydrolysates, peptone, yeast extract, ammonium sulfate, urea, and amino acids. Typically, the concentration of the nitrogen sources, in the culture medium is greater than about 0.1 g/L, preferably greater than about 0.25 g/L, and more preferably greater than about 1.0 g/L. Beyond certain concentrations, however, the addition of a nitrogen source to the culture medium is not advantageous for the growth of the microorganisms. As a result, the concentration of the nitrogen sources, in the culture medium is less than about 20 g/L, preferably less than about 10 g/L and more preferably less than about 5 g/L. Further, in some instances it may be desirable to allow the culture medium to become depleted of the nitrogen sources during culture.

The effective culture medium can contain other compounds such as inorganic salts, vitamins, trace metals, or growth promoters. Such other compounds can also be present in carbon, nitrogen, or mineral sources in the effective medium or can be added specifically to the medium.

The culture medium can also contain a suitable phosphate source. Such phosphate sources include both inorganic and organic phosphate sources. Preferred phosphate sources include, but are not limited to, phosphate salts such as mono or dibasic sodium and potassium phosphates, ammonium phosphate, and mixtures thereof. Typically, the concentration of phosphate in the culture medium is greater than about 1.0 g/L, preferably greater than about 2.0 g/L, and more preferably greater than about 5.0 g/L. Beyond certain concentrations, however, the addition of phosphate to the culture medium is not advantageous for the growth of the microorganisms. Accordingly, the concentration of phosphate in the culture medium is typically less than about 20 g/L, preferably less than about 15 g/L, and more preferably less than about 10 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, preferably greater than about 1.0 g/L, and more preferably greater than about 2.0 g/L. Beyond certain concentrations, however, the addition of magnesium to the culture medium is not advantageous for the growth of the microorganisms. Accordingly, the concentration of magnesium in the culture medium is typically less than about 10 g/L, preferably less than about 5 g/L, and more preferably less than about 3 g/L. Further, in some instances, it may be desirable to allow the culture medium to become depleted of a magnesium source during culture.

In some embodiments, the culture medium can also include a biologically acceptable chelating agent, such as the dihydrate of trisodium citrate. In such instance, the concentration of a chelating agent in the culture medium is greater than about 0.2 g/L, preferably greater than about 0.5 g/L, and more preferably greater than about 1 g/L. Beyond certain concentrations, however, the addition of a chelating agent to the culture medium is not advantageous for the growth of the microorganisms. Accordingly, the concentration of a chelating agent in the culture medium is typically less than about 10 g/L, preferably less than about 5 g/L, and more preferably less than about 2 g/L.

The culture medium can also initially include a biologically acceptable acid or base to maintain the desired pH of the culture medium. Biologically acceptable acids include, but are not limited to, hydrochloric acid, sulfuric acid, nitric acid, phosphoric acid, and mixtures thereof. Biologically acceptable bases include, but are not limited to, ammonium hydroxide, sodium hydroxide, potassium hydroxide, and mixtures thereof. In some embodiments, the base used is ammonium hydroxide.

The culture medium can also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, preferably within the range of from about 20 mg/L to about 1000 mg/L, and more preferably in the range of from about 50 mg/L to about 500 mg/L.

The culture medium can also include sodium chloride. Typically, the concentration of sodium chloride in the culture medium is within the range of from about 0.1 g/L to about 5 g/L, preferably within the range of from about 1 g/L to about 4 g/L, and more preferably in the range of from about 2 g/L to about 4 g/L.

In some embodiments, the culture medium can also include trace metals. Such trace metals can be added to the culture medium as a stock solution that, for convenience, can be prepared separately from the rest of the culture medium. Typically, the amount of such a trace metals solution added to the culture medium is greater than about 1 mL/L, preferably greater than about 5 mL/L, and more preferably greater than about 10 mL/L. Beyond certain concentrations, however, the addition of a trace metals to the culture medium is not advantageous for the growth of the microorganisms. Accordingly, the amount of such a trace metals solution added to the culture medium is typically less than about 100 mL/L, preferably less than about 50 mL/L, and more preferably less than about 30 mL/L. It should be noted that, in addition to adding trace metals in a stock solution, the individual components can be added separately, each within ranges corresponding independently to the amounts of the components dictated by the above ranges of the trace metals solution.

The culture medium can include other vitamins, such as pantothenate, biotin, calcium, pantothenate, inositol, pyridoxine-HCl, and thiamine-HCl. Such vitamins can be added to the culture medium as a stock solution that, for convenience, can be prepared separately from the rest of the culture medium. Beyond certain concentrations, however, the addition of vitamins to the culture medium is not advantageous for the growth of the microorganisms.

The culture medium may be supplemented with hexanoic acid or hexanoate as a precursor for the cannabinoid biosynthetic pathway. The hexanoic acid may have a concentration of less than 3 mM hexanoic acid (e.g., from 1 nM to 2.9 mM hexanoic acid, from 10 nM to 2.9 mM hexanoic acid, from 100 nM to 2.9 mM hexanoic acid, or from 1 μM to 2.9 mM hexanoic acid) hexanoic acid.

The fermentation methods described herein can be performed in conventional culture modes, which include, but are not limited to, batch, fed-batch, cell recycle, continuous and semi-continuous. In some embodiments, the fermentation is carried out in fed-batch mode. In such a case, some of the components of the medium are depleted during culture, including pantothenate during the production stage of the fermentation. In some embodiments, the culture may be supplemented with relatively high concentrations of such components at the outset, for example, of the production stage, so that growth and/or production is supported for a period of time before additions are required. The preferred ranges of these components are maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those in the art, the rate of consumption of nutrient increases during culture as the cell density of the medium increases. Moreover, to avoid introduction of foreign microorganisms into the culture medium, addition is performed using aseptic addition methods, as are known in the art. In addition, a small amount of anti-foaming agent may be added during the culture.

The temperature of the culture medium can be any temperature suitable for growth of the genetically modified cells and/or production of compounds of interest. For example, prior to inoculation of the culture medium with an inoculum, the culture medium can be brought to and maintained at a temperature in the range of from about 20° C. to about 45° C., preferably to a temperature in the range of from about 25° C. to about 40° C. and more preferably in the range of from about 28° C. to about 32° C.

The pH of the culture medium can be controlled by the addition of acid or base to the culture medium. In such cases when ammonia is used to control pH, it also conveniently serves as a nitrogen source in the culture medium. Preferably, the pH is maintained from about 3.0 to about 8.0, more preferably from about 3.5 to about 7.0, and most preferably from about 4.0 to about 6.5.

In some embodiments, the carbon source concentration, such as the glucose concentration, of the culture medium is monitored during culture. Glucose or sucrose concentration of the culture medium can be monitored using known techniques, such as, for example, use of the glucose oxidase enzyme test or high pressure liquid chromatography, which can be used to monitor glucose concentration in the supernatant, e.g., a cell-free component of the culture medium. As stated previously, the carbon source concentration should be kept below the level at which cell growth inhibition occurs. Although such concentration may vary from organism to organism, for glucose as a carbon source, cell growth inhibition occurs at glucose concentrations greater than at about 60 g/L and can be determined readily by trial. Accordingly, when glucose is used as a carbon source the glucose is preferably fed to the fermenter and maintained in the range of from about 1 g/L to about 100 g/L, more preferably in the range of from about 2 g/L to about 50 g/L, and yet more preferably in the range of from about 5 g/L to about 20 g/L. Alternatively, the glucose concentration in the culture medium is maintained below detection limits. Although the carbon source concentration can be maintained within desired levels by addition of, for example, a substantially pure glucose solution, it is acceptable, and may be preferred, to maintain the carbon source concentration of the culture medium by addition of aliquots of the original culture medium. The use of aliquots of the original culture medium may be desirable because the concentrations of other nutrients in the medium (e.g. the nitrogen and phosphate sources) can be maintained simultaneously. Likewise, the trace metals concentrations can be maintained in the culture medium by addition of aliquots of the trace metals solution.

### Examples

The following examples are put forth to provide those of ordinary skill in the art with a description of how the compositions and methods described herein may be used, made, and evaluated, and are intended to be purely exemplary of the invention and are not intended to limit the scope of what the inventors regard as their invention.

### Example 1: Transformation of Heterologous Nucleic Acids into Yeast Cells

Each DNA construct was integrated into Saccharomyces cerevisiae (CEN.PK113-7D) using standard molecular biology techniques in an optimized lithium acetate transformation. Briefly, cells were grown overnight in yeast extract peptone dextrose (YPD) medium at 30° C. with shaking (200 rpm), diluted to an OD600 of 0.1 in 100 mL YPD, and grown to an OD600 of 0.6-0.8. For each transformation, 5 mL of culture were harvested by centrifugation, washed in 5 mL of sterile water, spun down again, resuspended in 1 mL of 100 mM lithium acetate, and transferred to a microcentrifuge tube. Cells were spun down (13,000× g) for 30 s, the supernatant was removed, and the cells were resuspended in a transformation mix consisting of 240 μL 50% PEG, 36 μL 1 M lithium acetate, 10 μL boiled salmon sperm DNA, and 74 μL of donor DNA. For transformations that required expression of the endonuclease F-Cph1, the donor DNA included a plasmid carrying the F-CphI gene expressed under the yeast TDH3 promoter. F-CphI endonuclease expressed in such a manner cuts a specific recognition site engineered in a host strain to facilitate integration of the target gene of interest. Following a heat shock at 42° C. for 40 min, cells were recovered overnight in YPD medium before plating on selective medium. When applicable, DNA integration was confirmed by colony PCR with primers specific to the integrations.

### Example 2: Culturing of Yeast

For routine strain characterization in a 96-well-plate format, yeast colonies were picked into a 1.1-mL-per-well capacity 96-well ‘Pre-Culture plate’ filled with 360 μL per well of pre-culture medium. Pre-culture medium consisted of Bird Seed Media (BSM, originally described by van Hoek et al., Biotech. and Bioengin., 68, 2000, 517-23) at pH 5.05 with 14 g/L sucrose, 7 g/L maltose, 3.75 g/L ammonium sulfate, and 1 g/L lysine. Cells were cultured at 28° C. in a high capacity microtiter plate incubator shaking at 1000 rpm and 80% humidity for 3 days until the cultures reached carbon exhaustion.

The growth-saturated cultures were sub-cultured by taking 14.4 μL from the saturated cultures and diluting into a 2.2 mL per well capacity 96-well ‘production plate’ filled with 360 μL per well of production medium. Production medium consisted of BSM at pH 5.05 with 40 g/L sucrose, 3.75 g/L ammonium sulfate, and 2 mM hexanoic acid. Cells in the production medium were cultured at 30° C. in a high capacity microtiter plate shaker at 1000 rpm and 80% humidity for an additional 3 days prior to extraction and analysis.

### Example 3: Analytical Methods for Product Extraction and Titer Determination

Samples for olivetolic acid and cannabinoid measurements were initially analyzed in high-throughput by mass spectrometer (Agilent 6470-QQQ) with a RapidFire 365 system autosampler with C4 cartridge.

TABLE 1
RapidFire 365 System Configuration

| | | |
|---|---|---|
| Pump 1: 0.1% acetic acid in water | 0.8 | mL/min |
| Pump 2: 0.1% formic acid in acetonitrile | 1.5 | mL/min |
| Pump 3: 0.1% formic acid in 40% acetone in water | 0.8 | mL/min |
| State 1: Aspirate | 600 | ms |
| State 2: Load/Wash | 2000 | m |
| State 3: Extra wash | 500 | ms |
| State 4: Elute | 6000 | ms |
| State 5: Reequilibrate | 1000 | ms |

TABLE 2
6470-QQQ MS Method Configuration

| Ion Source | AJS ESI | |
|---|---|---|
| Time Filtering peak width | 0.02 | min |
| Stop Time | No limit/as pump | |
| Scan Type | MRM | |
| Diverter Valve | To MS | |
| Delta EMV | (+)0/(−)0 | |
| Ion Mode (polarity) | Negative | |
| Gas Temp | 300° | C. |
| Gas Flow | 13 | L/min |
| Nebulizer | 30 | psi |
| Sheath Gas Temp | 30° | C. |
| Sheath Gas Flow | 12 | L/min |
| Negative Capillary V | 3500 | V |

The peak areas from a chromatogram from a mass spectrometer were used to generate the calibration curve using authentic standards. The amount in moles of each compound were generated through external calibration using an authentic standard.

Hit samples from the initial screen were then analyzed for HTAL, PDAL, olivetol, olivetolic acid, CBGa, and CBDa on a weight per volume basis, by the method below. All measurements were performed by reverse phase ultra-high pressure liquid chromatography and ultraviolet detection (UPLC-UV) using Thermo Vanquish Flex Binary UHPLC System with a Vanquish Diode Array Detector HL.

TABLE 3
Mobile Phases and Column Information

| | |
|---|---|
| Mobile Phase A: | 99.9% water + 0.1% Formic Acid, 5 mM |
| | ammonium formate |
| Mobile Phase B: | 99.9% acetonitrile + 0.1% Formic acid |
| Column | Thermo Scientific Accucore Polar Premium C18 |
| | 100 mm × 2.1 mm × 2.6 um, Thermo P/N |
| | 28026-103030 |
| Guard Column | Thermo Scientific Guard Cartridge, 4 PK, P/N |
| | 28103014001 |
| Guard Column holder | Uniguard Holder, ThermoFisher, 4.0-4.6 mm, |
| | P/N 850-00 |

TABLE 4
Gradient Method

| Time | Flow [mL/min] | % A | % B | Curve |
|---|---|---|---|---|
| 0.00 | 1.2 | 70 | 30 | 5 |
| 1.00 | 1.2 | 20 | 80 | 5 |
| 1.75 | 1.2 | 12.5 | 87.5 | 5 |
| 1.80 | 1.2 | 70 | 30 | 5 |
| 2.1 | 1.2 | 70 | 30 | 5 |

TABLE 5
Autosampler Parameters

| Draw speed | 2.00 | ul/s |
|---|---|---|
| Dispense speed | 5.00 | ul/s |
| Injection wash mode | Both (before and after draw) | |
| Injection wash time | 5.0 | s |
| Injection wash speed | 10.0 | ul/s |
| Sample puncture - puncture offset | 0 | um |
| Temperature control | 8 | C. |

TABLE 6
Column Compartment Settings

| Temperature control | On | |
|---|---|---|
| Temperature | 50.0 | C. |
| Ready temp delta | 0.50 | C. |
| Equilibration time | 1.0 | min |

| | |
|---|---|
| Thermostatting mode | Still air |
| Fan Speed | 5 |

TABLE 7
Detector Settings

| | | |
|---|---|---|
| UV-Vis Channel 1 Wavelength | 270 | nm |
| Data collection rate | 50.0 | Hz |
| Response time | 0.010 | s |
| Peak width | 0.100 | min |

Analytes were identified by retention time compared to an authentic standard. The peak areas were used to generate the linear calibration curve for each analyte. At the conclusion of the incubation of the production plate, methanol was added to each well such that the final concentration was 67% (v/v) methanol. An impermeable seal was added, and the plate was shaken at 1000 rpm for 30 seconds to lyse the cells and extract cannabinoids. The plate was centrifuged for 30 seconds at 200× g to pellet cell debris. 300 μL of the clarified sample was moved to an empty 1.1-mL-capacity 96-well plate and sealed with a foil seal. The sample plate was stored at −20° C. until analysis.

### Example 4: Generation of a CBGa-Production Base Strain for CBDaS Screening

To screen for cannabidiolic acid (CBDa) production, a cannabigerolic acid (CBGa) production strain was constructed, as CBGa and molecular oxygen are the two substrates necessary for CBDa production. CBDa synthase (CBDaS) test constructs were then integrated into the CBGa production strain in a high-throughput fashion and screened for CBDa production.

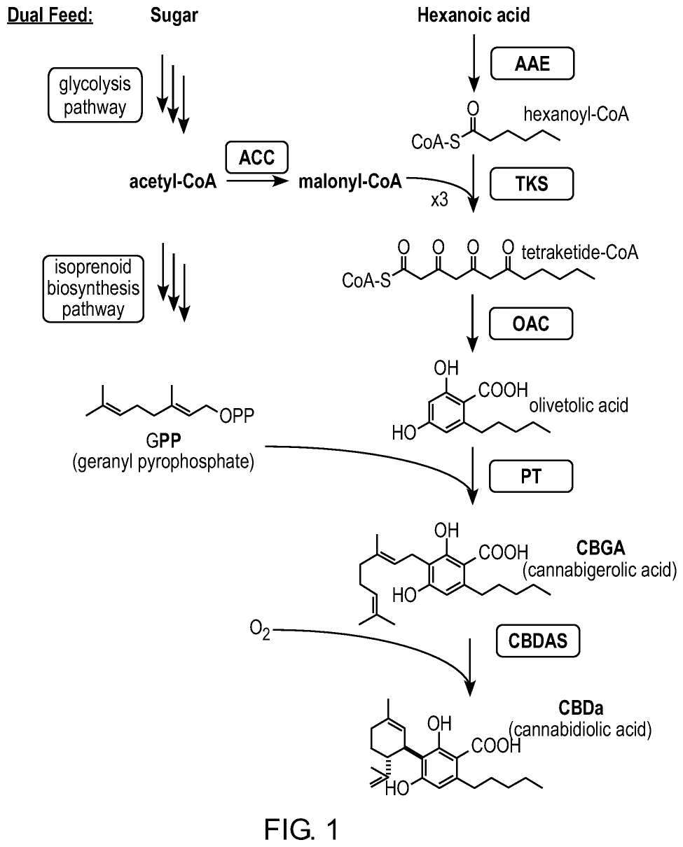

A CBGa production strain was created from the maltose-switchable Saccharomyces cerevisiae strain mentioned above by expressing the genes of the mevalonate pathway under the control of native GAL promoters. This strain comprised the following chromosomally integrated mevalonate pathway genes from S. cerevisiae: acetyl-CoA thiolase (ERG10), HMG-COA synthase (ERG13), HMG-COA reductase (HMGR), mevalonate kinase (ERG12), phosphomevalonate kinase (ERG8), mevalonate pyrophosphate decarboxylase (MVD1), and IPP: DMAPP isomerase (IDI1). In addition, the strain contained copies of five heterologous enzymes involved in the cannabinoid biosynthetic pathway (FIG. 1): the acyl-activating enzyme (AAE) (SEQ ID NO. 56), tetraketide synthase (TKS) (SEQ ID NO. 74), olivetolic acid cyclase (OAC) (SEQ ID NO. 102), and cannabigerolic acid synthase (CBGaS) from Stachybotrys chartarum (SEQ ID NO. 170), as well as geranylpyrophosphate synthase (GPPS) from Streptomyces aculeolatus (SEQ ID NO. 171), all under the control of GAL regulated promoters. To increase flux to cytosolic acetyl-CoA, PDC from Zymomonas mobilis, and overexpression of S. cerevisiae ALD6 and ACS1 were included in the engineering. All heterologous genes described herein were codon optimized for S. cerevisiae utilizing suitable algorithms. FIG. 1 shows a depiction of the biosynthetic pathway to CBGA utilized in the CBDaS screening strain.

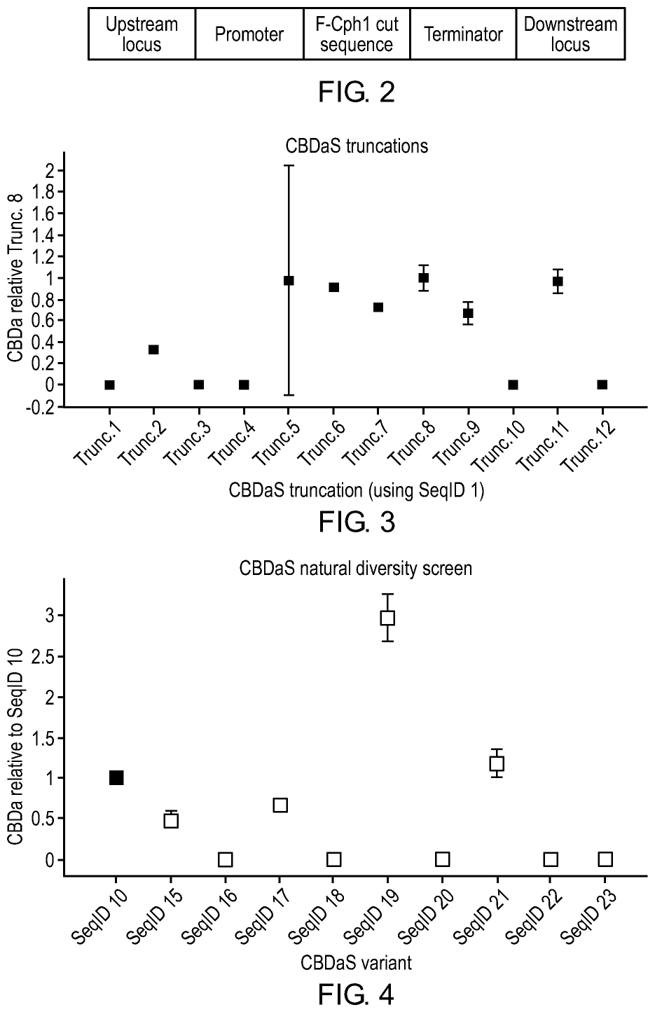

In order to screen the library of candidate genes for CBDaS activity, a “landing pad” approach was utilized (FIG. 2), as described in, for example, U.S. Pat. No. 7,919,605. An intergenic region in the screening strain was altered to contain an F-CphI endonuclease recognition site, which was flanked by a strong, GAL-regulon promoter and a terminator, both from yeast. This site allowed the candidate genes to be integrated into the genome by co-transformation of the endonuclease alongside donor DNA containing the desired DNA sequence to be screened, flanked by 40 base pair homology regions to the promoter and terminator. This CBGa-producer landing pad strain was used for all screening in the examples below.

### Example 5: Identification of High-Performing CBDaS Natural Diversity Variants

CBDaS enzymes (SEQ ID NO: 1) was used as the reference sequence. The PEP4 signal sequence from Komagataella pastoris (SEQ ID NO: 2) was fused to twelve versions of the CBDaS reference, each having different N-terminal truncations that removed the native Cannibis signal sequence (FIG. 3, Table 8). CBDa titers are reported in Table 8 below (CBD titers, although not routinely measured, were detected at low levels). The highest CBDaS activity was observed from Trunc. 8.

TABLE 8
Reference CBDaS Truncation Series Fused with
Komagataella pastoris PEP4 Signal Sequence

| | CBDa titer | | |
|---|---|---|---|
| Truncation | relative to | N-terminal | |
| ID | Trunc. 8 | truncation (#aa) | CBDaS SeqID |
| Trunc. 1 | 0.00 | Δ1-20 | SEQ ID NO: 3 |
| Trunc. 2 | 0.33 | Δ1-21 | SEQ ID NO: 4 |
| Trunc. 3 | 0.00 | Δ1-22 | SEQ ID NO: 5 |
| Trunc. 4 | 0.00 | Δ1-23 | SEQ ID NO: 6 |
| Trunc. 5 | 0.98 | Δ1-25 | SEQ ID NO: 7 |
| Trunc. 6 | 0.92 | Δ1-26 | SEQ ID NO: 8 |
| Trunc. 7 | 0.72 | Δ1-27 | SEQ ID NO: 9 |
| Trunc. 8 | 1.00 | Δ1-28 | SEQ ID NO: 10 |
| Trunc. 9 | 0.67 | Δ1-29 | SEQ ID NO: 11 |
| Trunc. 10 | 0.00 | Δ1-30 | SEQ ID NO: 12 |
| Trunc. 11 | 0.97 | Δ1-31 | SEQ ID NO: 13 |
| Trunc. 12 | 0.00 | Δ1-32 | SEQ ID NO: 14 |

The reference CBDaS was used as a BLAST query for UniParc. Nine additional naturally occurring CBDaS variants were identified from UniParc with >98% amino acid identity. All nine variants were screened using the A1-28aa truncation (Trunc. 8) fused to the PEP4 signal sequence from Komagataella pastoris (SEQ ID NO: 2) (FIG. 4, Table 9). CBDa titers are reported in Table 9 below (CBD titers, although not routinely measured, were detected at low levels). The highest CBDaS activity was observed from Div. Variant ID 6, which showed about 3-fold higher activity than the reference CBDaS.

TABLE 9
CBDaS Natural Diversity Variants

| | CBDa titer | | | |
|---|---|---|---|---|
| Diversity | relative to | | Mutations relative | |
| variant ID | Div. ID 1 | UniProt ID | to Div. ID 1 | SEQ ID NO |
| Div. ID 1 | 1.00 | A6P6V9 | (reference enzyme) | SEQ ID NO: 10 |
| Div. ID 2 | 0.48 | A0A0E3TJM6 | L539Q | SEQ ID NO: 15 |
| Div. ID 3 | 0.00 | A0A0E3TIL5 | P476S | SEQ ID NO: 16 |
| Div. ID 4 | 0.66 | A0A0E3XJ72 | H143R | SEQ ID NO: 17 |
| Div. ID 5 | 0.00 | A0A0E3TJM8 | P476S, L539Q | SEQ ID NO: 18 |
| Div. ID 6 | 2.97 | A0A0E3XIC7 | T74S, N168S, N196S, K474Q | SEQ ID NO: 19 |
| Div. ID 7 | 0.00 | A0A0E3TIM7 | T74S, N168S, N196S, K474Q, | SEQ ID NO: 20 |
| | | | G489R | |
| Div. ID 8 | 1.19 | A0A0E3XHS4 | T74S, N168S, N196S, G375R, | SEQ ID NO: 21 |
| | | | K474Q | |
| Div. ID 9 | 0.00 | A0A3G5EA56 | Y471H, K474Q, P476S, L481I | SEQ ID NO: 22 |
| Div. ID 10 | 0.00 | A0A3G5EBM5 | T74S, N168S, N196S, K474Q, | SEQ ID NO: 23 |
| | | | N495H, Y499P, Q501H, W505R, | |
| | | | G506A, E507Q, G511R, K512Q, | |
| | | | R516K | |

### Example 6: Basic Yeast Surface Display with CBDaS

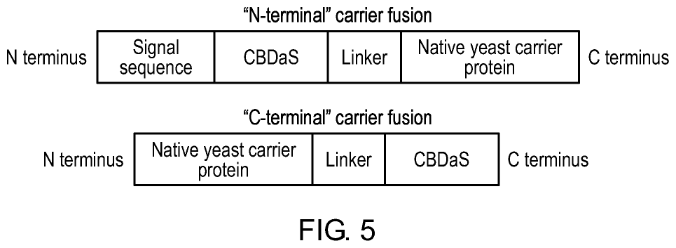

CBDaS requires low pH for activity (Zirpel et al., 2018, J. Biotechnol. 284:17-26). The cytoplasm is neutral pH and so not suitable for CBDa production, however yeast fermentation media is low pH. Yeast surface display is a method for covalently attaching proteins of interest to the outside of the yeast cell wall by fusion to native cell wall proteins (FIG. 5). By expressing CBDaS using a surface display construct, CBDaS will reside in a low pH environment optimal for activity, while still remaining cell associated.

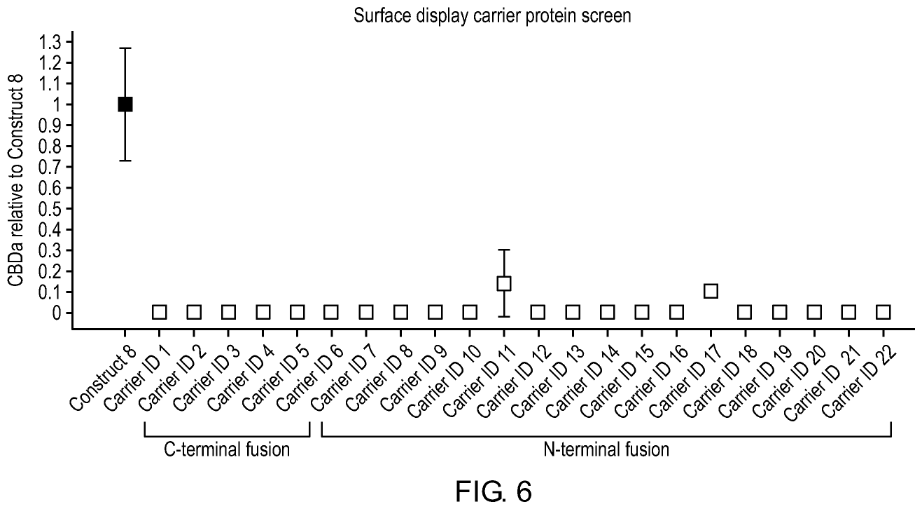

CBDaS was fused to a variety of native yeast cell wall proteins, called “carrier” proteins (FIG. 5, FIG. 6, Table 10). Two native yeast carrier proteins, SAG1 and FLO5, showed CBDaS activity when the reference CBDaS (SEQ ID NO: 1) was fused to the carrier's N-terminus, as shown in Table 10 below. The native signal sequence from S. cerevisiae AGA2 (SEQ ID NO: 42) and a short 6 aa flexible linker (SEQ ID NO: 113) were used to fuse FLO5 (SEQ ID NO: 34) and SAG1 (SEQ ID NO: 36) to CBDaS (Construct 32 and Construct 38, respectively).

TABLE 10
Surface Display Carrier Protein Screen

| | | | | | Fushion | |
|---|---|---|---|---|---|---|
| | CBDa | | | | type | |
| Carrier | relative to | | | Carrier | (to carrier | |
| protein | Construct | Gene | | protein | N or C | Construct |
| ID | 8 | name | Uniprot | truncation | terminus) | ID |
| Carrier ID 1 | 0.00 | FLO1 | P32768 | Δ1100-1537 | C-terminus | Construct 22 |
| Carrier ID 2 | 0.00 | PIR1 | Q03178 | | C-terminus | Construct 23 |
| Carrier ID 3 | 0.00 | PIR2 | P32478 | | C-terminus | Construct 24 |
| Carrier ID 4 | 0.00 | PIR3 | Q03180 | | C-terminus | Construct 25 |
| Carrier ID 5 | 0.00 | PIR4 | P47001 | | C-terminus | Construct 26 |
| Carrier ID 6 | 0.00 | AGA1 | P32323 | Δ1-150 | N-terminus | Construct 27 |
| Carrier ID 7 | 0.00 | CCW12 | Q12127 | Δ1-60 | N-terminus | Construct 28 |
| Carrier ID 8 | 0.00 | CWP1 | P28319 | Δ1-26 | N-terminus | Construct 29 |
| Carrier ID 9 | 0.00 | CWP2 | P43497 | Δ1-25 | N-terminus | Construct 30 |
| Carrier ID 10 | 0.00 | DAN4 | P47179 | Δ1-760 | N-terminus | Construct 31 |
| Carrier ID 11 | 0.14 | FLO5 | P38894 | Δ1-658 | N-terminus | Construct 32 |
| | | | (S1002N, | | | |
| | | | M1015K, | | | |
| | | | S1040Y) | | | |
| Carrier ID 12 | 0.00 | PIR1 | Q03178 | | N-terminus | Construct 33 |
| Carrier ID 13 | 0.00 | PIR2 | P32478 | | N-terminus | Construct 34 |
| Carrier ID 14 | 0.00 | PIR3 | Q03180 | | N-terminus | Construct 35 |
| Carrier ID 15 | 0.00 | PIR4 | P47001 | | N-terminus | Construct 36 |
| Carrier ID 16 | 0.00 | PRY3 | P47033 | Δ1-800 | N-terminus | Construct 37 |
| Carrier ID 17 | 0.10 | SAG1 | P20840 | Δ1-330 | N-terminus | Construct 38 |
| Carrier ID 18 | 0.00 | SED1 | Q01589 | Δ1-109 | N-terminus | Construct 39 |
| Carrier ID 19 | 0.00 | SRP2 | P33890 | Δ1-155 | N-terminus | Construct 40 |
| Carrier ID 20 | 0.00 | TIP1 | P27654 | Δ1-66 | N-terminus | Construct 41 |
| Carrier ID 21 | 0.00 | TIR1 | P10863 | Δ1-42 | N-terminus | Construct 42 |
| Carrier ID 22 | 0.00 | TOS6 | P48560 | Δ1-37 | N-terminus | Construct 43 |
| Construct 8 | 1.00 | | | | | Construct 8 |
| (reference) | | | | | | |

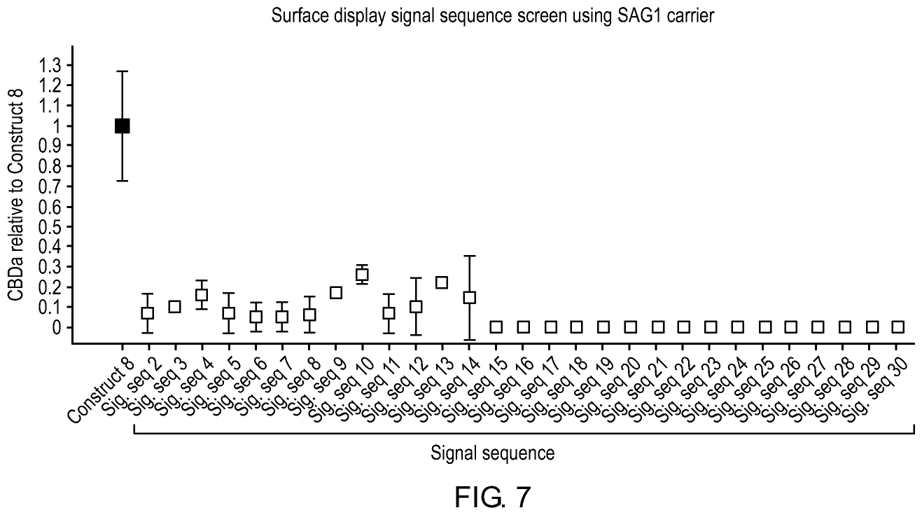

Alternate yeast signal sequences were tested in place of the AGA2 signal sequence in the SAG1 surface display construct (Construct 38). Twelve additional signal sequences showed activity, up to ˜2.5-fold more activity than AGA2 (FIG. 7, Table 11). CBDa titers are reported in Table 11 below (CBD titers, although not routinely measured, were detected at low levels).

TABLE 11
Surface Display Signal Sequence Screen Using SAG1 as a Carrier Protein

| Signal | CBDa titer | Source | Source | Signal | |
|---|---|---|---|---|---|
| sequence | relative to | gene | gene | sequence | Construct |
| ID | Construct 8 | name | UniProt ID | SeqID | ID |
| Sig. seq 2 | 0.07 | AGA1 | P32323 | SeqID 43 | Construct 44 |
| Sig. seq 3 | 0.10 | AGA2 | P32781 | SeqID 42 | Construct 38 |
| (used previously) | | | | | |
| Sig. seq 4 | 0.16 | CWP2 | P43497 | SeqID 44 | Construct 46 |
| Sig. seq 5 | 0.07 | CCW12 | Q12127 | SeqID 45 | Construct 47 |
| Sig. seq 6 | 0.05 | PIR1 | Q03178 | SeqID 46 | Construct 48 |
| Sig. seq 7 | 0.05 | PIR3 | Q03180 | SeqID 47 | Construct 49 |
| Sig. seq 8 | 0.06 | SRP2 | P33890 | SeqID 48 | Construct 50 |
| Sig. seq 9 | 0.17 | K28 | Q7LZU3 | SeqID 49 | Construct 51 |
| Sig. seq 10 | 0.26 | BAR1 | P12630 | SeqID 50 | Construct 52 |
| Sig. seq 11 | 0.07 | DAN4 | P47179 | SeqID 51 | Construct 53 |
| Sig. seq 12 | 0.10 | OST1 | P41543 | SeqID 52 | Construct 54 |
| Sig. seq 13 | 0.22 | SUC2 | P00724 | SeqID 53 | Construct 55 |
| Sig. seq 14 | 0.15 | PEP4 | P07267 | SeqID 54 | Construct 56 |
| Sig. seq 15 | 0.00 | CWP1 | P28319 | SeqID 55 | Construct 57 |
| Sig. seq 16 | 0.00 | PIR2 | P32478 | SeqID 57 | Construct 58 |
| Sig. seq 17 | 0.00 | PIR4 | P47001 | SeqID 58 | Construct 59 |
| Sig. seq 18 | 0.00 | TIP1 | P27654 | SeqID 59 | Construct 60 |
| Sig. seq 19 | 0.00 | SED1 | Q01589 | SeqID 60 | Construct 61 |
| Sig. seq 20 | 0.00 | TIR1 | P10863 | SeqID 61 | Construct 62 |
| Sig. seq 21 | 0.00 | PRY3 | P47033 | SeqID 62 | Construct 63 |
| Sig. seq 22 | 0.00 | TOS6 | P48560 | SeqID 63 | Construct 64 |
| Sig. seq 23 | 0.00 | K1 | A0A076FME7 | SeqID 64 | Construct 65 |
| Sig. seq 24 | 0.00 | DAN1 | P47178 | SeqID 65 | Construct 66 |
| Sig. seq 25 | 0.00 | MF(ALPHA)1 | P01149 | SeqID 66 | Construct 67 |
| Sig. seq 26 | 0.00 | PRC1 | P00729 | SeqID 67 | Construct 68 |
| Sig. seq 27 | 0.00 | HPF1 | Q05164 | SeqID 68 | Construct 69 |
| Sig. seq 28 | 0.00 | SCW10 | Q04951 | SeqID 69 | Construct 70 |
| Sig. seq 29 | 0.00 | PGU1 | P47180 | SeqID 70 | Construct 71 |
| Sig. seq 30 | 0.00 | SAG1 | P20840 | SeqID 71 | Construct 72 |
| Construct 8 | 1.00 | | | | |
| (reference) | | | | | |

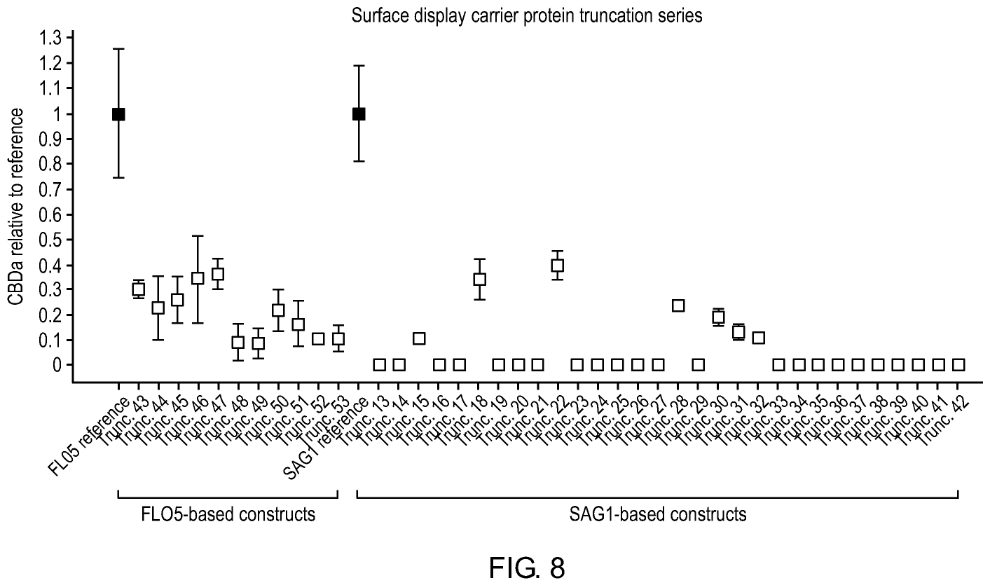

Alternate truncations of both SAG1 and FLO5 were tested with the AGA2 signal sequence (SEQ ID NO: 42) and short 6 aa flexible linker (SEQ ID NO: 113), using the reference CBDaS (SEQ ID NO: 1) for SAG1, and the alternate CBDaS natural diversity variant for FLO5 (SEQ ID NO: 136) (FIG. 8, Table 12). Multiple variants of both SAG1 and FLOS showed improved activity. CBDa titers are reported in Table 12 below (CBD titers, although not routinely measured, were detected at low levels).

TABLE 12
Surface Display Carrier Protein Truncation Series

| | CBDa | | | | |
|---|---|---|---|---|---|
| | titer | N | Carrier | Carrier | |
| Truncation | relative to | terminal | protein | protein | Construct |
| ID | reference | truncation | name | SeqID | ID |
| Trunc. 13 | 0.00 | Δ1-321 | SAG1 | SeqID 72 | Construct 73 |
| Trunc. 14 | 0.00 | Δ1-329 | SAG1 | SeqID 73 | Construct 74 |
| Trunc. 15 | 0.10 | Δ1-330 | SAG1 | SeqID 36 | Construct 38 |
| (original | | | | | |
| SAG1) | | | | | |
| Trunc. 16 | 0.00 | Δ1-338 | SAG1 | SeqID 75 | Construct 76 |
| Trunc. 17 | 0.00 | Δ1-349 | SAG1 | SeqID 76 | Construct 77 |
| Trunc. 18 | 0.34 | Δ1-359 | SAG1 | SeqID 77 | Construct 78 |
| Trunc. 19 | 0.00 | Δ1-369 | SAG1 | SeqID 78 | Construct 79 |
| Trunc. 20 | 0.00 | Δ1-383 | SAG1 | SeqID 79 | Construct 80 |
| Trunc. 21 | 0.00 | Δ1-389 | SAG1 | SeqID 80 | Construct 81 |
| Trunc. 22 | 0.40 | Δ1-399 | SAG1 | SeqID 81 | Construct 82 |
| Trunc. 23 | 0.00 | Δ1-409 | SAG1 | SeqID 82 | Construct 83 |
| Trunc. 24 | 0.00 | Δ1-419 | SAG1 | SeqID 83 | Construct 84 |
| Trunc. 25 | 0.00 | Δ1-429 | SAG1 | SeqID 84 | Construct 85 |
| Trunc. 26 | 0.00 | Δ1-439 | SAG1 | SeqID 85 | Construct 86 |
| Trunc. 27 | 0.00 | Δ1-449 | SAG1 | SeqID 86 | Construct 87 |
| Trunc. 28 | 0.24 | Δ1-459 | SAG1 | SeqID 87 | Construct 88 |
| Trunc. 29 | 0.00 | Δ1-469 | SAG1 | SeqID 88 | Construct 89 |
| Trunc. 30 | 0.19 | Δ1-479 | SAG1 | SeqID 89 | Construct 90 |
| Trunc. 31 | 0.13 | Δ1-489 | SAG1 | SeqID 90 | Construct 91 |
| Trunc. 32 | 0.11 | Δ1-499 | SAG1 | SeqID 91 | Construct 92 |
| Trunc. 33 | 0.00 | Δ1-509 | SAG1 | SeqID 92 | Construct 93 |
| Trunc. 34 | 0.00 | Δ1-519 | SAG1 | SeqID 93 | Construct 94 |
| Trunc. 35 | 0.00 | Δ1-529 | SAG1 | SeqID 94 | Construct 95 |
| Trunc. 36 | 0.00 | Δ1-539 | SAG1 | SeqID 95 | Construct 96 |
| Trunc. 37 | 0.00 | Δ1-549 | SAG1 | SeqID 96 | Construct 97 |
| Trunc. 38 | 0.00 | Δ1-559 | SAG1 | SeqID 97 | Construct 98 |
| Trunc. 39 | 0.00 | Δ1-569 | SAG1 | SeqID 98 | Construct 99 |
| Trunc. 40 | 0.00 | Δ1-579 | SAG1 | SeqID 99 | Construct 100 |
| Trunc. 41 | 0.00 | Δ1-589 | SAG1 | SeqID 100 | Construct 101 |
| Trunc. 42 | 0.00 | Δ1-599 | SAG1 | SeqID 101 | Construct 102 |
| SAG1 | 1.00 | | | none | Construct 8 |
| experimental | | | | | |
| control | | | | | |
| Trunc. 43 | 0.30 | Δ1-658 | FLO5 | SeqID 34 | Construct 103 |
| (original | | | | | |
| FLO5) | | | | | |
| Trunc. 44 | 0.23 | Δ1-659 | FLO5 | SeqID 103 | Construct 104 |
| Trunc. 45 | 0.26 | Δ1-660 | FLO5 | SeqID 104 | Construct 105 |
| Trunc. 46 | 0.34 | Δ1-661 | FLO5 | SeqID 105 | Construct 106 |
| Trunc. 47 | 0.36 | Δ1-662 | FLO5 | SeqID 106 | Construct 107 |
| Trunc. 48 | 0.09 | Δ1-671 | FLO5 | SeqID 107 | Construct 108 |
| Trunc. 49 | 0.09 | Δ1-681 | FLO5 | SeqID 108 | Construct 109 |
| Trunc. 50 | 0.22 | Δ1-691 | FLO5 | SeqID 109 | Construct 110 |
| Trunc. 51 | 0.16 | Δ1-701 | FLO5 | SeqID 110 | Construct 111 |
| Trunc. 52 | 0.11 | Δ1-711 | FLO5 | SeqID 111 | Construct 112 |
| Trunc. 53 | 0.11 | Δ1-711 | FLO5 | SeqID 112 | Construct 113 |
| FLO5 | 1.00 | | | none | Construct 17 |
| experimental | | | | | |
| control | | | | | |

### Example 7: Optimized Yeast Surface Display Constructs

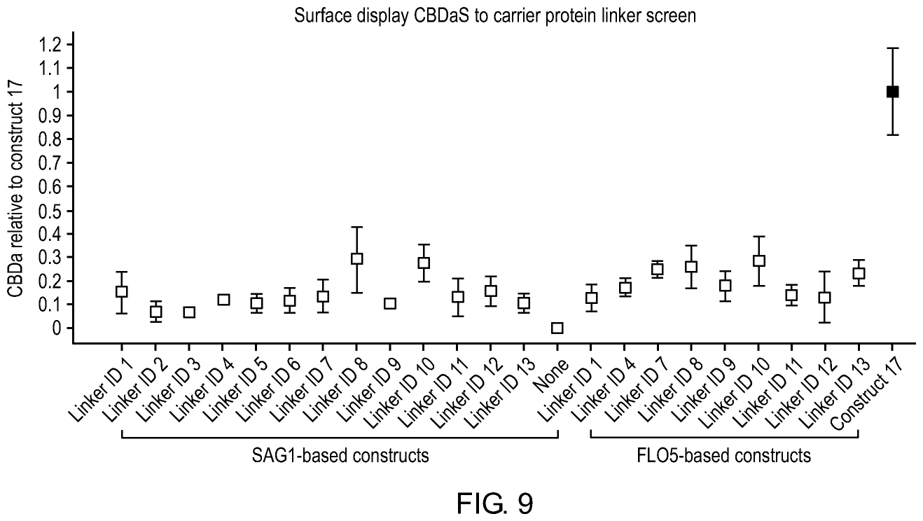

The SAG1 and FLO5 yeast surface display CBDaS expression constructs were further optimized. Twelve additional linkers were tested in both SAG1 and FLO5 CBDaS expression constructs. (Table 13). All the linker carrier protein combinations were functional except for a no-linker control (FIG. 9, Table 14). Long rigid linkers were the top performers, giving up to about 2-fold improvements over the original 6 aa flexible linker (SEQ ID NO: 113) for both SAG1 and FLO5 (Constructs 121 and 132, respectively). CBDa titers are reported in Table 14 below (CBD titers, although not routinely measured, were detected at low levels).

TABLE 13
Linkers

| | | | Linker | |
|---|---|---|---|---|
| | | Linker | length | Linker |
| Linker ID | Linker aa seq. | type | (aa) | SEQ ID NO |
| Linker ID 1 | GSGGSG | flexible | 6 | SEQ ID NO: 113 |
| (original) | | | | |
| Linker ID 2 | GSGSGS | flexible | 6 | SEQ ID NO: 114 |
| Linker ID 3 | HHHHGSGGSG | flexible | 10 | SEQ ID NO: 115 |
| Linker ID 4 | GSGAGGVSGAGG | flexible | 12 | SEQ ID NO: 116 |
| Linker ID 5 | GSGGSGGSGGSG | flexible | 12 | SEQ ID NO: 117 |
| Linker ID 6 | HHHHHHGSGGSG | flexible | 12 | SEQ ID NO: 118 |
| Linker ID 7 | GSGGSGGSGGSGGSGGSG | flexible | 18 | SEQ ID NO: 119 |
| Linker ID 8 | AEAAAKEAAAKA | rigid | 12 | SEQ ID NO: 120 |
| Linker ID 9 | APAPAPAPAPAPAPA | rigid | 15 | SEQ ID NO: 121 |
| Linker ID 10 | EPEPEPEPEPEPEPE | rigid | 15 | SEQ ID NO: 122 |
| Linker ID 11 | KPKPKPKPKPKPKP | rigid | 14 | SEQ ID NO: 123 |
| Linker ID 12 | AEAAAKEAAAKEAAAKA | rigid | 17 | SEQ ID NO: 124 |
| Linker ID 13 | AEAAAKEAAAKEAAAKEAAAKA | rigid | 22 | SEQ ID NO: 125 |

TABLE 14
Surface Display CBDaS to Carrier Protein Linker Screen

| | CBDa | | | | |
|---|---|---|---|---|---|
| | relative to | Linker | Linker | | Construct |
| Linker ID | construct 17 | type | length | Carrier | ID |
| None | 0.00 | | | SAG1 | Construct 114 |
| Linker ID 1 | 0.15 | flexible | 6 | SAG1 | Construct 38 |
| Linker ID 2 | 0.07 | flexible | 6 | SAG1 | Construct 115 |
| Linker ID 3 | 0.07 | flexible | 10 | SAG1 | Construct 116 |
| Linker ID 4 | 0.12 | flexible | 12 | SAG1 | Construct 117 |
| Linker ID 5 | 0.10 | flexible | 12 | SAG1 | Construct 118 |
| Linker ID 6 | 0.12 | flexible | 12 | SAG1 | Construct 119 |
| Linker ID 7 | 0.13 | flexible | 18 | SAG1 | Construct 120 |
| Linker ID 8 | 0.29 | rigid | 12 | SAG1 | Construct 121 |
| Linker ID 9 | 0.10 | rigid | 15 | SAG1 | Construct 122 |
| Linker ID 10 | 0.27 | rigid | 15 | SAG1 | Construct 123 |
| Linker ID 11 | 0.13 | rigid | 15 | SAG1 | Construct 124 |
| Linker ID 12 | 0.15 | rigid | 17 | SAG1 | Construct 125 |
| Linker ID 13 | 0.10 | rigid | 22 | SAG1 | Construct 126 |
| Linker ID 1 | 0.13 | flexible | 6 | FLO5 | Construct 127 |
| Linker ID 4 | 0.17 | flexible | 12 | FLO5 | Construct 128 |
| Linker ID 7 | 0.25 | flexible | 18 | FLO5 | Construct 129 |
| Linker ID 8 | 0.26 | rigid | 12 | FLO5 | Construct 130 |
| Linker ID 9 | 0.18 | rigid | 15 | FLO5 | Construct 131 |
| Linker ID 10 | 0.28 | rigid | 15 | FLO5 | Construct 132 |
| Linker ID 11 | 0.14 | rigid | 15 | FLO5 | Construct 133 |
| Linker ID 12 | 0.13 | rigid | 17 | FLO5 | Construct 134 |
| Linker ID 13 | 0.23 | rigid | 22 | FLO5 | Construct 135 |
| Construct 17 | 1.00 | | | none | Construct 17 |

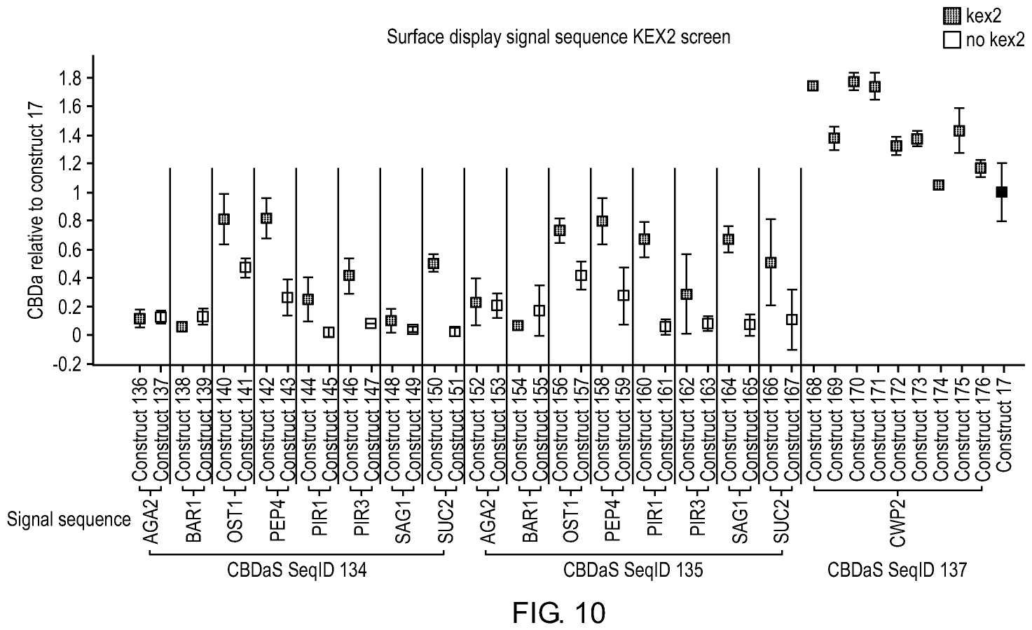

KEX2 protease recognition sites were introduced between the signal sequence and the N-terminus of CBDaS in surface display expression constructs to force removal of the signal sequence. KEX2 (UniProt P13134) is a native S. cerevisiae processing protease that resides in the Golgi, and has a specific amino acid recognition sequence of (Lys/Arg)-Arg. Multiple variants of the KEX2 recognition sequence were tested (FIG. 10, Table 15, Table 16). Addition of KEX2 recognition sites improved CBDaS activity, even when paired with different signal sequences and different CBDaS N-terminal truncations. CBDa titers are reported in Table 16 below (CBD titers, although not routinely measured, were detected at low levels).

TABLE 15
KEX2 Protease Recognition Sequences Tested
KEX2 protease recognition sequences

RR
KR
RRK
RRQ
RRW
RRE
LDKR
LDKREAEA
KREAEA

TABLE 16
Surface Display Signal Sequence KEX2 Protease Site Screen

| | | | | Signal | CBDaS |
|---|---|---|---|---|---|
| | CBDa titer | Signal | KEX2 | sequence | N-terminal |
| | relative to | sequence | site | SEQ ID | truncation |
| Construct ID | construct 17 | name | (aa) | NO | SEQ ID NO |
| Construct 136 | 0.11 | AGA2 | RR | SEQ ID NO: 126 | SEQ ID NO: 134 |
| Construct 137 | 0.12 | AGA2 | | SEQ ID NO: 42 | SEQ ID NO: 134 |
| Construct 138 | 0.06 | BAR1 | RR | SEQ ID NO: 127 | SEQ ID NO: 134 |
| Construct 139 | 0.13 | BAR1 | | SEQ ID NO: 50 | SEQ ID NO: 134 |
| Construct 140 | 0.81 | OST1 | RR | SEQ ID NO: 128 | SEQ ID NO: 134 |
| Construct 141 | 0.47 | OST1 | | SEQ ID NO: 52 | SEQ ID NO: 134 |
| Construct 142 | 0.82 | PEP4 | RR | SEQ ID NO: 129 | SEQ ID NO: 134 |
| Construct 143 | 0.26 | PEP4 | | SEQ ID NO: 54 | SEQ ID NO: 134 |
| Construct 144 | 0.25 | PIR1 | RR | SEQ ID NO: 130 | SEQ ID NO: 134 |
| Construct 145 | 0.02 | PIR1 | | SEQ ID NO: 46 | SEQ ID NO: 134 |
| Construct 146 | 0.41 | PIR3 | RR | SEQ ID NO: 131 | SEQ ID NO: 134 |
| Construct 147 | 0.08 | PIR3 | | SEQ ID NO: 47 | SEQ ID NO: 134 |
| Construct 148 | 0.10 | SAG1 | RR | SEQ ID NO: 132 | SEQ ID NO: 134 |
| Construct 149 | 0.04 | SAG1 | | SEQ ID NO: 71 | SEQ ID NO: 134 |
| Construct 150 | 0.50 | SUC2 | RR | SEQ ID NO: 133 | SEQ ID NO: 134 |
| Construct 151 | 0.02 | SUC2 | | SEQ ID NO: 53 | SEQ ID NO: 134 |
| Construct 152 | 0.23 | AGA2 | RR | SEQ ID NO: 123 | SEQ ID NO: 135 |
| Construct 153 | 0.21 | AGA2 | | SEQ ID NO: 42 | SEQ ID NO: 135 |
| Construct 154 | 0.06 | BAR1 | RR | SEQ ID NO: 127 | SEQ ID NO: 135 |
| Construct 155 | 0.17 | BAR1 | | SEQ ID NO: 50 | SEQ ID NO: 135 |
| Construct 156 | 0.73 | OST1 | RR | SEQ ID NO: 128 | SEQ ID NO: 135 |
| Construct 157 | 0.42 | OST1 | | SEQ ID NO: 52 | SEQ ID NO: 135 |
| Construct 158 | 0.80 | PEP4 | RR | SEQ ID NO: 129 | SEQ ID NO: 135 |
| Construct 159 | 0.27 | PEP4 | | SEQ ID NO: 54 | SEQ ID NO: 135 |
| Construct 160 | 0.67 | PIR1 | RR | SEQ ID NO: 130 | SEQ ID NO: 135 |
| Construct 161 | 0.05 | PIR1 | | SEQ ID NO: 46 | SEQ ID NO: 135 |
| Construct 162 | 0.29 | PIR3 | RR | SEQ ID NO: 131 | SEQ ID NO: 135 |
| Construct 163 | 0.08 | PIR3 | | SEQ ID NO: 47 | SEQ ID NO: 135 |
| Construct 164 | 0.67 | SAG1 | RR | SEQ ID NO: 132 | SEQ ID NO: 135 |
| Construct 165 | 0.07 | SAG1 | | SEQ ID NO: 71 | SEQ ID NO: 135 |
| Construct 166 | 0.51 | SUC2 | RR | SEQ ID NO: 133 | SEQ ID NO: 135 |
| Construct 167 | 0.11 | SUC2 | | SEQ ID NO: 53 | SEQ ID NO: 135 |
| Construct 168 | 1.74 | CWP2 | RR | SEQ ID NO: 138 | SEQ ID NO: 137 |
| Construct 169 | 1.38 | CWP2 | KR | SEQ ID NO: 139 | SEQ ID NO: 137 |
| Construct 170 | 1.77 | CWP2 | RRK | SEQ ID NO: 140 | SEQ ID NO: 137 |
| Construct 171 | 1.74 | CWP2 | RRQ | SEQ ID NO: 141 | SEQ ID NO: 137 |
| Construct 172 | 1.32 | CWP2 | RRW | SEQ ID NO: 142 | SEQ ID NO: 137 |
| Construct 173 | 1.37 | CWP2 | RRE | SEQ ID NO: 143 | SEQ ID NO: 137 |
| Construct 174 | 1.05 | CWP2 | LDKR | SEQ ID NO: 144 | SEQ ID NO: 137 |
| Construct 175 | 1.05 | CWP2 | LDKREAEA | SEQ ID NO: 145 | SEQ ID NO: 137 |
| Construct 175 | 1.16 | CWP2 | KREAEA | SEQ ID NO: 146 | SEQ ID NO: 137 |
| Construct 17 | 1.00 | | | | |

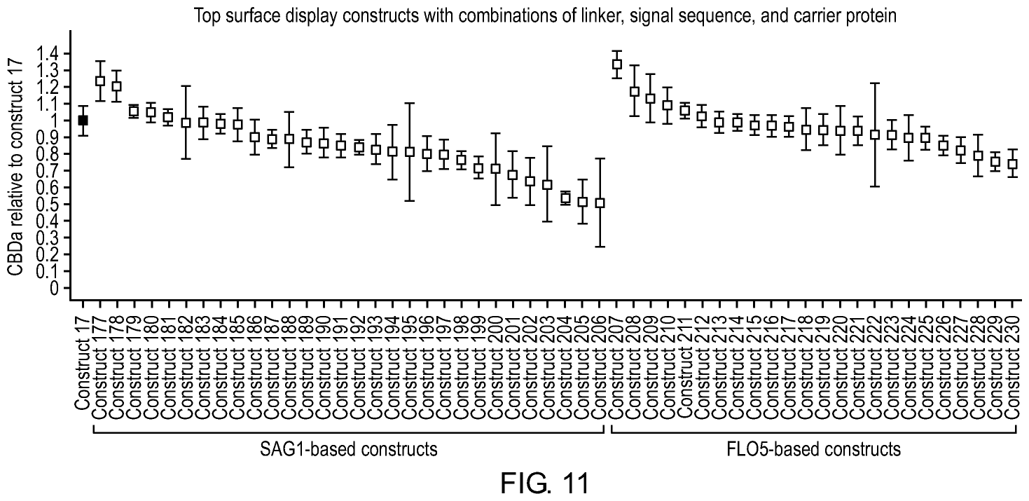

A variety of the top SAG1 and FLOS carrier protein truncations, signal sequences, KEX2 protease sites, CBDaS N-terminal truncations, and linkers were combinatorially tested (FIG. 11, Table 17). CBDa titers are shown in Table 17 below (CBD titers, although not routinely measured, were detected at low levels).

TABLE 17
Example Optimized Surface Display Constructs with Combinations
of Linker, Signal Sequence, and Carrier Protein

| | CBDa relative | | | | Carrier | Carrier |
|---|---|---|---|---|---|---|
| | to Construct | Signal | | | protein | protein |
| Construct ID | 17 | sequence | KEX2 | Linker ID | name | truncation |
| Construct 17 | 1.00 | | | | | |
| (reference) | | | | | | |
| Construct 177 | 1.24 | OST1 | RR | Linker ID 10 | SAG1 | Δ1-329 |
| Construct 178 | 1.20 | CWP2 | RR | Linker ID 8 | SAG1 | Δ1-329 |
| Construct 179 | 1.06 | CWP2 | RR | Linker ID 10 | SAG1 | Δ1-359 |
| Construct 180 | 1.05 | OST1 | RR | Linker ID 10 | SAG1 | Δ1-359 |
| Construct 181 | 1.02 | PEP4 | | Linker ID 10 | SAG1 | Δ1-459 |
| Construct 182 | 0.99 | OST1 | RR | Linker ID 10 | SAG1 | Δ1-459 |
| Construct 183 | 0.99 | AGA2 | RR | Linker ID 10 | SAG1 | Δ1-359 |
| Construct 184 | 0.98 | PEP4 | RR | Linker ID 10 | SAG1 | Δ1-359 |
| Construct 185 | 0.98 | PEP4 | | Linker ID 10 | SAG1 | Δ1-359 |
| Construct 186 | 0.91 | CCW1 | | Linker ID 10 | SAG1 | Δ1-399 |
| Construct 187 | 0.89 | CCW1 | | Linker ID 8 | SAG1 | Δ1-399 |
| Construct 188 | 0.89 | SUC2 | RR | Linker ID 10 | SAG1 | Δ1-359 |
| Construct 189 | 0.87 | AGA2 | RR | Linker ID 10 | SAG1 | Δ1-329 |
| Construct 190 | 0.87 | CWP2 | RR | Linker ID 10 | SAG1 | Δ1-329 |
| Construct 191 | 0.85 | CCW1 | | Linker ID 10 | SAG1 | Δ1-459 |
| Construct 192 | 0.84 | AGA2 | RR | Linker ID 10 | SAG1 | Δ1-399 |
| Construct 193 | 0.83 | CWP2 | RR | Linker ID 10 | SAG1 | Δ1-399 |
| Construct 194 | 0.82 | CWP2 | RR | Linker ID 10 | SAG1 | Δ1-459 |
| Construct 195 | 0.82 | OST1 | RR | Linker ID 10 | SAG1 | Δ1-399 |
| Construct 196 | 0.80 | PEP4 | | Linker ID 10 | SAG1 | Δ1-399 |
| Construct 197 | 0.80 | AGA2 | RR | Linker ID 8 | SAG1 | Δ1-399 |
| Construct 198 | 0.77 | CWP2 | RR | Linker ID 8 | SAG1 | Δ1-399 |
| Construct 199 | 0.72 | PEP4 | RR | Linker ID 10 | SAG1 | Δ1-329 |
| Construct 200 | 0.71 | OST1 | RR | Linker ID 10 | SAG1 | Δ1-359 |
| Construct 201 | 0.68 | OST1 | RR | Linker ID 10 | SAG1 | Δ1-399 |
| Construct 202 | 0.64 | OST1 | RR | Linker ID 10 | SAG1 | Δ1-329 |
| Construct 203 | 0.62 | OST1 | RR | Linker ID 10 | SAG1 | Δ1-459 |
| Construct 204 | 0.54 | PEP4 | | Linker ID 10 | SAG1 | Δ1-329 |
| Construct 205 | 0.52 | SUC2 | RR | Linker ID 10 | SAG1 | Δ1-459 |
| Construct 206 | 0.39 | SUC2 | RR | Linker ID 10 | SAG1 | Δ1-399 |
| Construct 207 | 1.33 | OST1 | RR | Linker ID 10 | FLO5 | Δ1-671 |
| Construct 208 | 1.18 | PEP4 | RR | Linker ID 10 | FLO5 | Δ1-691 |
| Construct 209 | 1.13 | OST1 | | Linker ID 10 | FLO5 | Δ1-671 |
| Construct 210 | 1.09 | AGA2 | RR | Linker ID 8 | FLO5 | Δ1-691 |
| Construct 211 | 1.06 | OST1 | RR | Linker ID 10 | FLO5 | Δ1-691 |
| Construct 212 | 1.02 | OST1 | | Linker ID 10 | FLO5 | Δ1-691 |
| Construct 213 | 0.99 | OST1 | RR | Linker ID 8 | FLO5 | Δ1-658 |
| Construct 214 | 0.99 | OST1 | RR | Linker ID 10 | FLO5 | Δ1-691 |
| Construct 215 | 0.97 | OST1 | RR | Linker ID 8 | FLO5 | Δ1-691 |
| Construct 216 | 0.97 | OST1 | RR | Linker ID 10 | FLO5 | Δ1-671 |
| Construct 217 | 0.97 | OST1 | | Linker ID 8 | FLO5 | Δ1-691 |
| Construct 218 | 0.95 | OST1 | RR | Linker ID 8 | FLO5 | Δ1-691 |
| Construct 219 | 0.95 | DAN4 | | Linker ID 10 | FLO5 | Δ1-671 |
| Construct 220 | 0.94 | OST1 | | Linker ID 8 | FLO5 | Δ1-658 |
| Construct 221 | 0.94 | DAN4 | | Linker ID 8 | FLO5 | Δ1-691 |
| Construct 222 | 0.92 | DAN4 | | Linker ID 10 | FLO5 | Δ1-691 |
| Construct 223 | 0.92 | AGA2 | RR | Linker ID 10 | FLO5 | Δ1-691 |
| Construct 224 | 0.90 | AGA2 | RR | Linker ID 10 | FLO5 | Δ1-671 |
| Construct 225 | 0.90 | OST1 | RR | Linker ID 8 | FLO5 | Δ1-658 |
| Construct 226 | 0.85 | AGA2 | RR | Linker ID 8 | FLO5 | Δ1-658 |
| Construct 227 | 0.83 | PEP4 | RR | Linker ID 8 | FLO5 | Δ1-691 |
| Construct 228 | 0.79 | PEP4 | RR | Linker ID 10 | FLO5 | Δ1-671 |
| Construct 229 | 0.75 | PEP4 | RR | Linker ID 8 | FLO5 | Δ1-658 |
| Construct 230 | 0.75 | DAN4 | | Linker ID 8 | FLO5 | Δ1-658 |

### Example 8: CBDaS Secretion and Vacuolar Localization

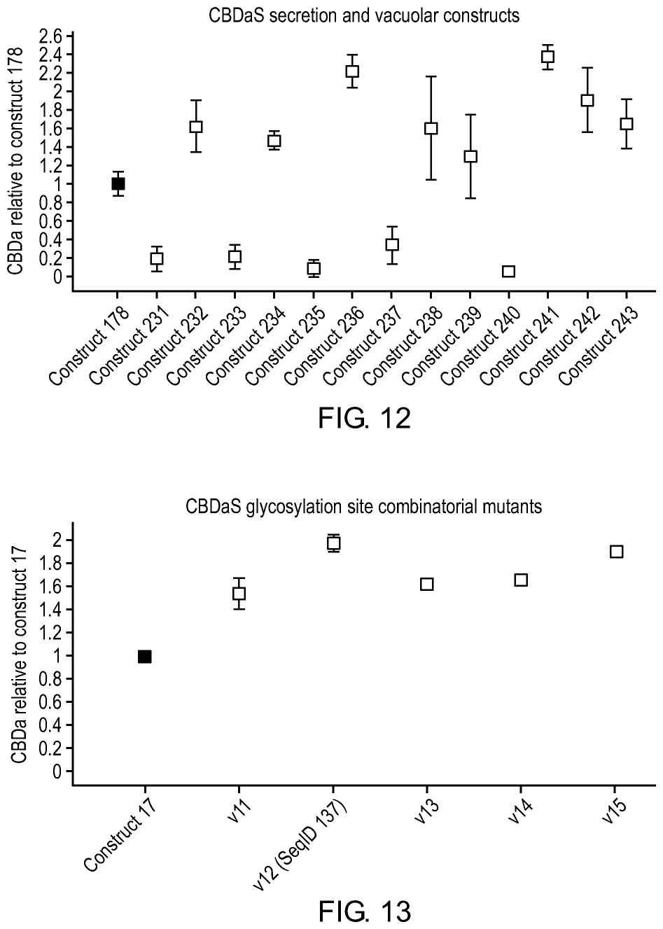

An alternative to yeast surface display constructs for CBDaS activity in the extracellular environment (Example 6) is direct secretion into the media. A series of constructs were tested 5 using the native S. cerevisiae mating factor alpha (MFα) pre sequence (signal sequence) (FIG. 12, Table 18). MFα secretion constructs were tested with both the native MFα pro sequence (SEQ ID NO: 153) (Constructs 231-234), as well as 2 artificial pro sequences from Kjeldsen et al., 2001, Biotech. Genet. Eng. Rev., 18:89-121 (SEQ ID NO: 154 and SEQ ID NO: 155) (Constructs 235-238). Surface display constructs that lacked the surface display carrier 10 protein were tested as well (Constructs 241-243). As the vacuole is a low pH environment within the cell, and PEP4 is a highly abundant native S. cerevisiae vacuolar protein, fusions to S. cerevisiae PEP4 (SEQ ID NO: 156) (Construct 240) or just the S. cerevisiae PEP4 pre-pro sequences (SEQ ID NO: 157) (Construct 239) were also tested. CBDa titers for these constructs are shown in Table 18 below (CBD titers, although not routinely measured, were detected at low levels).

TABLE 18
CBDa Secretion and Vacuolar Constructs

| | CBDa | | Signal | CBDaS N | CBDaS C | CBDaS C- |
|---|---|---|---|---|---|---|
| | relative to | Signal | sequence | terminal | terminal | terminal |
| Construct ID | Construct 178 | sequence | SeqID | truncation | truncation | fusion |
| Construct 231 | 0.19 | MF(alpha)- | SeqID 153 | | | |
| | | prepro | | | | |
| Construct 232 | 1.65 | MF(alpha)- | SeqID 153 | Δ1-28 | | |
| | | prepro | | | | |
| Construct 233 | 1.65 | MF(alpha)- | SeqID 153 | | Δ544 | SeqID 152 |
| | | prepro | | | | |
| Construct 234 | 1.47 | MF(alpha)- | SeqID 153 | Δ1-28 | Δ544 | SeqID 152 |
| | | prepro | | | | |
| Construct 235 | 0.08 | MF(alpha)-pre, | SeqID 154 | | Δ544 | SeqID 152 |
| | | synthetic pro 1 | | | | |
| Construct 236 | 2.21 | MF(alpha)-pre, | SeqID 154 | Δ1-28 | Δ544 | SeqID 152 |
| | | synthetic pro 1 | | | | |
| Construct 237 | 0.34 | MF(alpha)-pre, | SeqID 155 | | Δ544 | SeqID 152 |
| | | synthetic pro 2 | | | | |
| Construct 238 | 1.60 | MF(alpha)-pre, | SeqID 155 | Δ1-28 | Δ544 | SeqID 152 |
| | | synthetic pro 2 | | | | |
| Construct 239 | 1.30 | PEP4-prepro | SeqID 157 | Δ1-28 | | |
| Construct 240 | 0.05 | PEP4 whole | SeqID 156 | Δ1-28 | | |
| | | protein | | | | |
| Construct 241 | 2.36 | CWP2 + KEX2 | SeqID 138 | Δ1-28 | | SeqID 120 |
| | | (RR) | | | | |
| Construct 242 | 1.91 | CWP2 + KEX2 | SeqID 138 | Δ1-28 | | |
| | | (RR) | | | | |
| Construct 243 | 1.65 | CWP2 + KEX2 | SeqID 138 | | Δ544 | SeqID 152 |
| | | (RR) | | | | |
| Construct 178 | 1.00 | | SeqID 138 | | | |
| (reference) | | | | | | |

### Example 9: CBDaS Glycosylation Site Mutations

The reference CBDaS (SEQ ID NO: 1) is predicted to be N-glycosylated at 7 positions in Cannabis. It is likely that glycosylation occurs at these sites in S. cerevisiae as well, as the Asn-(any aa except Pro)-(Thr or Ser)N-glycosylation recognition sequence is conserved between plants and fungi. However, the exact nature and extent of glycosylation is likely to be different between the two hosts, and over-glycosylation is a common problem for heterologous proteins expressed in S. cerevisiae.

The 7 predicted CBDas glycosylation sites were combinatorially mutagenized (FIG. 13, Table 19, Table 20) to either completely eliminate glycosylation (Asn->Gln), or alter the degree of glycosylation (Thr->Ser or Ser->Thr). SEQ ID NO: 19 was used as the parent CBDaS enzyme in Construct 17, which uses the optimal N-terminal CBDaS truncation identified in Example 5. For consistency, the amino acid numbering corresponds to untruncated CBDaS (SEQ ID NO: 136). SEQ ID NO: 136 has a mutation at N168 that eliminates glycosylation at that site, so the library was used to combinatorially restore the N168 glycosylation site. The results of these mutations are shown in Table 20 below, with some mutants showing up to 2-fold greater activity than the parent (CBD titers, although not routinely measured, were detected at low levels).

TABLE 19
CBDaS Glycosylation Site Locations Targeted for
Random Mutagenesis (Amino Acid Positions are
With Reference to SEQ ID NO: 1)

| CBDaS | | Alternate |
|---|---|---|
| glycosylation site | Glycosylation | recognition |
| position (aa) | site knockout | site |
| N45 | N45Q | T47S |
| N65 | N65Q | T67S |
| N168 | N168Q | S170T |
| N296 | N296Q | T298S |
| N304 | N304Q | T306S |
| N328 | N328Q | S330T |
| N498 | N498Q | T500S |

TABLE 20
CBDaS Glycosylation Site Combinatorial Mutants (All Variants
were Expressed in a Construct Identical to Construct 17)

| CBDaS | Average | Mutations relative | CBDaS |
|---|---|---|---|
| variant ID | CBDa | to SeqID 136 | truncation |
| v11 | 1.54 | T47S, S168N, S170T, N304Q | Δ1-28 |
| v12 | 1.97 | S168N, S170T, S330T | Δ1-28 |
| (SeqID 137) | | | |
| v13 | 1.62 | T47S, S168N, S170T, T500S | Δ1-28 |
| v14 | 1.66 | T67S, S168N, S170T, N296Q, | Δ1-28 |
| | | S330T, T500S | |
| v15 | 1.90 | T47S, T67S, S168N, S170T, | Δ1-28 |
| | | N304Q, S330T, T500S | |
| Construct 17 | 1.00 | | |

### Example 10: CBDaS Point Mutants

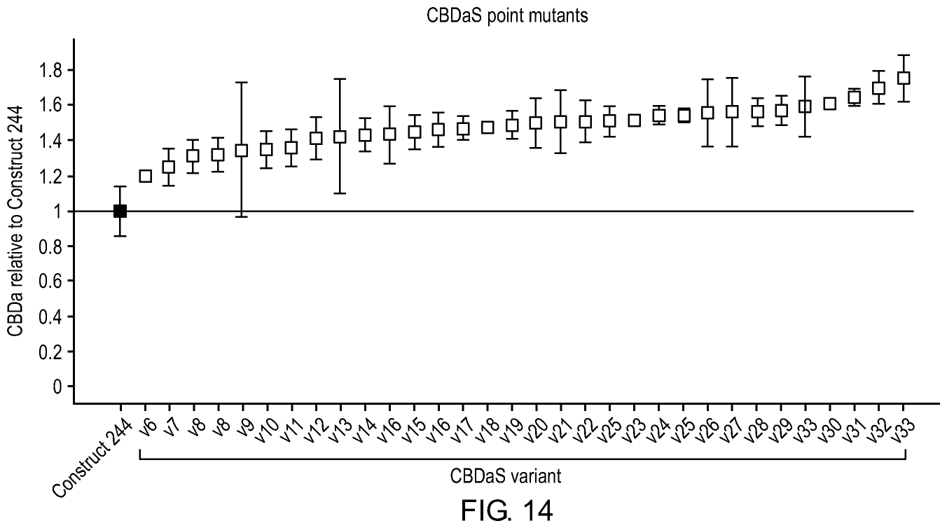

Site saturation mutagenesis was used to improve CBDaS activity (FIG. 14, Table 21). Each position in CBDaS SEQ ID NO: 137 was mutated using the degenerate codon NNT (where N can encode any of the 4 nucleotides) and transformed separately. The degenerate codon NNT can code for 15 different amino acids (A, C, D, F, G, H, I, L, N, P, R, S, T, V, and Y). Multiple isolates from each transformation were screened to accumulate data on multiple substitutions at each position. Mutagenesis was performed on a top surface display variant (Construct 244). CBDaS activity is shown below in Table 21, with some variants showing improved activity up to about 1.75 fold higher than the starting enzyme (CBD titers, although not routinely measured, were detected at low levels).

TABLE 21
Example CBDaS Point Mutants (All Variants
Were Expressed in a Construct Identical to Construct 244)

| | CBDa | | |
|---|---|---|---|
| | relative to | Mutant relative | Target |
| Variant ID | Construct 244 | to SeqID 137 | position |
| v11 | 0.10 | N29G | 29 |
| v13 | 0.32 | R31T | 31 |
| v15 | 0.10 | P43D | 43 |
| v17 | 0.07 | L49D | 49 |
| v20 | 0.14 | N56D | 56 |
| v26 | 0.19 | N57D | 57 |
| v9 | 0.38 | L71D | 71 |
| v21 | 0.18 | L71S | 71 |
| v32 | 0.09 | G95A | 95 |
| v8 | 0.10 | V103Y | 103 |
| v30 | 0.04 | V125D | 125 |
| v33 | 0.13 | I129L | 129 |
| v6 | 0.02 | H143A | 143 |
| v12 | 0.12 | W161R | 16 |
| v14 | 0.09 | W161A | 16 |
| v29 | 0.08 | W161H | 161 |
| v28 | 0.08 | W161D | 161 |
| v24 | 0.05 | W161S | 161 |
| v25 | 0.04 | W161T | 161 |
| v23 | 0.04 | W161N | 161 |
| v22 | 0.12 | H213N | 213 |
| v19 | 0.08 | H213D | 213 |
| v27 | 0.19 | I241V | 241 |
| v31 | 0.05 | K303N | 303 |
| v18 | 0.02 | S314C | 314 |
| v7 | 0.11 | T339S | 339 |
| v10 | 0.10 | F396L | 396 |
| v16 | 0.10 | V518C | 518 |
| SEQID 137 | 1.00 | | |

### Example 11: CBDaS Combinatorial Library Mutants

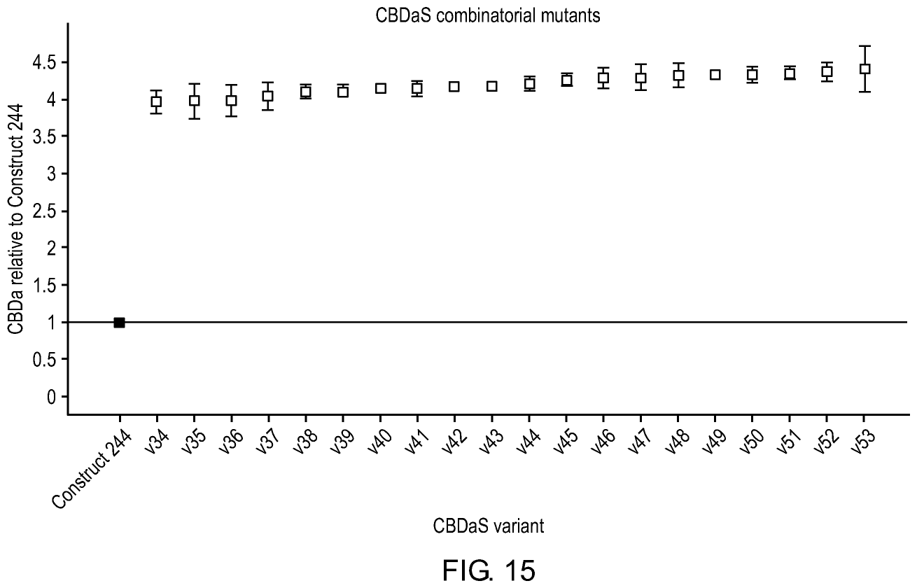

The top individual CBDaS point mutants from Example 10 were consolidated together using a full factorial combinatorial library (Table 22) to produce variants with far higher activity than any single CBDaS point mutant. Mutations were introduced into SEQ ID NO: 137 using PCR, and variants were expressed in a top surface display expression construct (Construct 244). The majority of point mutant combinations led to improved CBDaS activity over the parent (FIG. 15, Table 23), with quite a few variants showing activity greater than 4-fold over the parent, as shown in Table 23 below (CBD titers, although not routinely measured, were detected at low levels).

TABLE 22
CBDaS Positions Included in a
Combinatorial Library
CBDaS substitution (aa)

R53T
P65D
L71D
N78D
N79D
L93D
G117A
V147D
I151L
W183N
H235D
I263V
K325N
S336C
V540C

TABLE 23
Example CBDaS Combinatorial Mutants (All Variants Were Expressed
in a Construct Identical to Construct 244)

| | CBDa relative | |
|---|---|---|
| Seq ID | to SEQ | Mutations relative to |
| | ID NO: 137 | SEQ ID NO: 137 |
| v34 | 3.96 | R53T, N78D, V147D, H235D, I263V, K325N, V540C |
| v35 | 3.98 | R53T, P65D, L71D, N79D, L93D, V147D, W183N, H235D, S336C |
| v36 | 3.98 | L71D, N78D, G117A, V147D, W183N, I263V, K325N, S336C, V540C |
| v37 | 4.05 | R53T, P65D, L71D, N78D, N79D, L93D, V147D, W183N, H235D, |
| | | K325N, S336C, V540C |
| v38 | 4.11 | L71D, L93D, V147D, H235D, I263V |
| v39 | 4.11 | R35T, V147D, I151L, W183N, H235D, S336C, V540C |
| v40 | 4.14 | R53T, N78D, N79D, G117A, V147D, S336C |
| v41 | 4.14 | R53T, P65D, L71D, N79D, L93D, V147D, W183N, H235D, S336C |
| v42 | 4.17 | R53T, L71D, N78D, G117A, V147D, H235D, S336C, V540C |
| v43 | 4.18 | R53T, P65D, N78D, G117A, V147D, H325D, K325N, S336C, V540C |
| v44 | 4.21 | R53T, P65D, N78D, L93D, V147D, W183N, H235D, V540C |
| v45 | 4.26 | R53T, N78D, V147D, W183N, H235D, I263V, S336C |
| v46 | 4.29 | R53T, N79D, V147D, W183N, H235D, I263V, K325N, S336C |
| v47 | 4.29 | R53T, P65D, L71D, N78D, V147D.H235D, I263V, S336C, V540C |
| v48 | 4.32 | R53T, L71D, G117A, V147D, H235D, I263V, V540C |
| v49 | 4.33 | R53T, L71D, N78D, G117A, V147D, H235D, I263V, K325N, S336C, |
| | | V540C |
| v50 | 4.33 | R53T, P65D, N78D, N79D, V147D, S336C, V540C |
| v51 | 4.36 | R53T, N78D, N79D, V147D, W183N, H235D, I263V, K325N |
| v52 | 4.38 | R53T, I151L, H235D, K325N, S336C |
| v53 | 4.41 | R53T, P65D, L71D, N79D, L93D, V147D, W183N, H235D, S336C |
| SeqID | 1.00 | |
| 137 | | |

TABLE 24
Construct Table

| | SEQ IDs fused | | | SEQ ID fused |
|---|---|---|---|---|
| | upstream of | | SEQ ID fused | downstream of |
| | CBDaS (in | | downstream of | CBDaS (carrier |
| Construct ID | order) | CBDaS SeqID | CBDaS (linker) | protein) |
| Construct 1 | SeqID 2 | SeqID 3 | | |
| Construct 2 | SeqID 2 | SeqID 4 | | |
| Construct 3 | SeqID 2 | SeqID 5 | | |
| Construct 4 | SeqID 2 | SeqID 6 | | |
| Construct 5 | SeqID 2 | SeqID 7 | | |
| Construct 6 | SeqID 2 | SeqID 8 | | |
| Construct 7 | SeqID 2 | SeqID 9 | | |
| Construct 8 | SeqID 2 | SeqID 10 | | |
| Construct 9 | SeqID 2 | SeqID 11 | | |
| Construct 10 | SeqID 2 | SeqID 12 | | |
| Construct 11 | SeqID 2 | SeqID 13 | | |
| Construct 12 | SeqID 2 | SeqID 14 | | |
| Construct 13 | SeqID 2 | SeqID 15 | | |
| Construct 14 | SeqID 2 | SeqID 16 | | |
| Construct 15 | SeqID 2 | SeqID 17 | | |
| Construct 16 | SeqID 2 | SeqID 18 | | |
| Construct 17 | SeqID 2 | SeqID 19 | | |
| Construct 18 | SeqID 2 | SeqID 20 | | |
| Construct 19 | SeqID 2 | SeqID 21 | | |
| Construct 20 | SeqID 2 | SeqID 22 | | |
| Construct 21 | SeqID 2 | SeqID 23 | | |
| | SeqID 24, | | | |
| Construct 22 | SeqID 113 | SeqID 1 | SeqID 113 | |
| | SeqID 25, | | | |
| Construct 23 | SeqID 113 | SeqID 1 | SeqID 113 | |
| | SeqID 26, | | | |
| Construct 24 | SeqID 113 | SeqID 1 | SeqID 113 | |
| Construct 25 | SeqID 27, | SeqID 1 | SeqID 113 | |
| | SeqID 113 | | | |
| | SeqID 28, | | | |
| Construct 26 | SeqID 113 | SeqID 1 | SeqID 113 | |
| Construct 27 | SeqID 42 | SeqID 1 | SeqID 113 | SeqID 29 |
| Construct 28 | SeqID 42 | SeqID 1 | SeqID 113 | SeqID 30 |
| Construct 29 | SeqID 42 | SeqID 1 | SeqID 113 | SeqID 31 |
| Construct 30 | SeqID 42 | SeqID 1 | SeqID 113 | SeqID 32 |
| Construct 31 | SeqID 42 | SeqID 1 | SeqID 113 | SeqID 33 |
| Construct 32 | SeqID 42 | SeqID 1 | SeqID 113 | SeqID 34 |
| Construct 33 | SeqID 42 | SeqID 1 | SeqID 113 | SeqID 25 |
| Construct 34 | SeqID 42 | SeqID 1 | SeqID 113 | SeqID 26 |
| Construct 35 | SeqID 42 | SeqID 1 | SeqID 113 | SeqID 27 |
| Construct 36 | SeqID 42 | SeqID 1 | SeqID 113 | SeqID 28 |
| Construct 37 | SeqID 42 | SeqID 1 | SeqID 113 | SeqID 35 |
| Construct 38 | SeqID 42 | SeqID 1 | SeqID 113 | SeqID 36 |
| Construct 39 | SeqID 42 | SeqID 1 | SeqID 113 | SeqID 37 |
| Construct 40 | SeqID 42 | SeqID 1 | SeqID 113 | SeqID 38 |
| Construct 41 | SeqID 42 | SeqID 1 | SeqID 113 | SeqID 39 |
| Construct 42 | SeqID 42 | SeqID 1 | SeqID 113 | SeqID 40 |
| Construct 43 | SeqID 42 | SeqID 1 | SeqID 113 | SeqID 41 |
| Construct 44 | SeqID 43 | SeqID 1 | SeqID 113 | SeqID 36 |
| Construct 46 | SeqID 44 | SeqID 1 | SeqID 113 | SeqID 36 |
| Construct 47 | SeqID 45 | SeqID 1 | SeqID 113 | SeqID 36 |
| Construct 48 | SeqID 46 | SeqID 1 | SeqID 113 | SeqID 36 |
| Construct 49 | SeqID 47 | SeqID 1 | SeqID 113 | SeqID 36 |
| Construct 50 | SeqID 48 | SeqID 1 | SeqID 113 | SeqID 36 |
| Construct 51 | SeqID 49 | SeqID 1 | SeqID 113 | SeqID 36 |
| Construct 52 | SeqID 50 | SeqID 1 | SeqID 113 | SeqID 36 |
| Construct 53 | SeqID 51 | SeqID 1 | SeqID 113 | SeqID 36 |
| Construct 54 | SeqID 52 | SeqID 1 | SeqID 113 | SeqID 36 |
| Construct 55 | SeqID 53 | SeqID 1 | SeqID 113 | SeqID 36 |
| Construct 56 | SeqID 54 | SeqID 1 | SeqID 113 | SeqID 36 |
| Construct 57 | SeqID 55 | SeqID 1 | SeqID 113 | SeqID 36 |
| Construct 58 | SeqID 57 | SeqID 1 | SeqID 113 | SeqID 36 |
| Construct 59 | SeqID 58 | SeqID 1 | SeqID 113 | SeqID 36 |
| Construct 60 | SeqID 59 | SeqID 1 | SeqID 113 | SeqID 36 |
| Construct 61 | SeqID 60 | SeqID 1 | SeqID 113 | SeqID 36 |
| Construct 62 | SeqID 61 | SeqID 1 | SeqID 113 | SeqID 36 |
| Construct 63 | SeqID 62 | SeqID 1 | SeqID 113 | SeqID 36 |
| Construct 64 | SeqID 63 | SeqID 1 | SeqID 113 | SeqID 36 |
| Construct 65 | SeqID 64 | SeqID 1 | SeqID 113 | SeqID 36 |
| Construct 66 | SeqID 65 | SeqID 1 | SeqID 113 | SeqID 36 |
| Construct 67 | SeqID 66 | SeqID 1 | SeqID 113 | SeqID 36 |
| Construct 68 | SeqID 67 | SeqID 1 | SeqID 113 | SeqID 36 |
| Construct 69 | SeqID 68 | SeqID 1 | SeqID 113 | SeqID 36 |
| Construct 70 | SeqID 69 | SeqID 1 | SeqID 113 | SeqID 36 |
| Construct 71 | SeqID 70 | SeqID 1 | SeqID 113 | SeqID 36 |
| Construct 72 | SeqID 71 | SeqID 1 | SeqID 113 | SeqID 36 |
| Construct 73 | SeqID 42 | SeqID 1 | SeqID 113 | SeqID 72 |
| Construct 74 | SeqID 42 | SeqID 1 | SeqID 113 | SeqID 73 |
| Construct 76 | SeqID 42 | SeqID 1 | SeqID 113 | SeqID 75 |
| Construct 77 | SeqID 42 | SeqID 1 | SeqID 113 | SeqID 76 |
| Construct 78 | SeqID 42 | SeqID 1 | SeqID 113 | SeqID 77 |
| Construct 79 | SeqID 42 | SeqID 1 | SeqID 113 | SeqID 78 |
| Construct 80 | SeqID 42 | SeqID 1 | SeqID 113 | SeqID 79 |
| Construct 81 | SeqID 42 | SeqID 1 | SeqID 113 | SeqID 80 |
| Construct 82 | SeqID 42 | SeqID 1 | SeqID 113 | SeqID 81 |
| Construct 83 | SeqID 42 | SeqID 1 | SeqID 113 | SeqID 82 |
| Construct 84 | SeqID 42 | SeqID 1 | SeqID 113 | SeqID 83 |
| Construct 85 | SeqID 42 | SeqID 1 | SeqID 113 | SeqID 84 |
| Construct 86 | SeqID 42 | SeqID 1 | SeqID 113 | SeqID 85 |
| Construct 87 | SeqID 42 | SeqID 1 | SeqID 113 | SeqID 86 |
| Construct 88 | SeqID 42 | SeqID 1 | SeqID 113 | SeqID 87 |
| Construct 89 | SeqID 42 | SeqID 1 | SeqID 113 | SeqID 88 |
| Construct 90 | SeqID 42 | SeqID 1 | SeqID 113 | SeqID 89 |
| Construct 91 | SeqID 42 | SeqID 1 | SeqID 113 | SeqID 90 |
| Construct 92 | SeqID 42 | SeqID 1 | SeqID 113 | SeqID 91 |
| Construct 93 | SeqID 42 | SeqID 1 | SeqID 113 | SeqID 92 |
| Construct 94 | SeqID 42 | SeqID 1 | SeqID 113 | SeqID 93 |
| Construct 95 | SeqID 42 | SeqID 1 | SeqID 113 | SeqID 94 |
| Construct 96 | SeqID 42 | SeqID 1 | SeqID 113 | SeqID 95 |
| Construct 97 | SeqID 42 | SeqID 1 | SeqID 113 | SeqID 96 |
| Construct 98 | SeqID 42 | SeqID 1 | SeqID 113 | SeqID 97 |
| Construct 99 | SeqID 42 | SeqID 1 | SeqID 113 | SeqID 98 |
| Construct 100 | SeqID 42 | SeqID 1 | SeqID 113 | SeqID 99 |
| Construct 101 | SeqID 42 | SeqID 1 | SeqID 113 | SeqID 100 |
| Construct 102 | SeqID 42 | SeqID 1 | SeqID 113 | SeqID 101 |
| Construct 103 | SeqID 42 | SeqID 136 | SeqID 113 | SeqID 34 |
| Construct 104 | SeqID 42 | SeqID 136 | SeqID 113 | SeqID 103 |
| Construct 105 | SeqID 42 | SeqID 136 | SeqID 113 | SeqID 104 |
| Construct 106 | SeqID 42 | SeqID 136 | SeqID 113 | SeqID 105 |
| Construct 107 | SeqID 42 | SeqID 136 | SeqID 113 | SeqID 106 |
| Construct 108 | SeqID 42 | SeqID 136 | SeqID 113 | SeqID 107 |
| Construct 109 | SeqID 42 | SeqID 136 | SeqID 113 | SeqID 108 |
| Construct 110 | SeqID 42 | SeqID 136 | SeqID 113 | SeqID 109 |
| Construct 111 | SeqID 42 | SeqID 136 | SeqID 113 | SeqID 110 |
| Construct 112 | SeqID 42 | SeqID 136 | SeqID 113 | SeqID 111 |
| Construct 113 | SeqID 42 | SeqID 136 | SeqID 113 | SeqID 112 |
| Construct 114 | SeqID 42 | SeqID 1 | | SeqID 36 |
| Construct 115 | SeqID 42 | SeqID 1 | SeqID 114 | SeqID 36 |
| Construct 116 | SeqID 42 | SeqID 1 | SeqID 115 | SeqID 36 |
| Construct 117 | SeqID 42 | SeqID 1 | SeqID 116 | SeqID 36 |
| Construct 118 | SeqID 42 | SeqID 1 | SeqID 117 | SeqID 36 |
| Construct 119 | SeqID 42 | SeqID 1 | SeqID 118 | SeqID 36 |
| Construct 120 | SeqID 42 | SeqID 1 | SeqID 119 | SeqID 36 |
| Construct 121 | SeqID 42 | SeqID 1 | SeqID 120 | SeqID 36 |
| Construct 122 | SeqID 42 | SeqID 1 | SeqID 121 | SeqID 36 |
| Construct 123 | SeqID 42 | SeqID 1 | SeqID 122 | SeqID 36 |
| Construct 124 | SeqID 42 | SeqID 1 | SeqID 123 | SeqID 36 |
| Construct 125 | SeqID 42 | SeqID 1 | SeqID 124 | SeqID 36 |
| Construct 126 | SeqID 42 | SeqID 1 | SeqID 125 | SeqID 36 |
| Construct 127 | SeqID 42 | SeqID 1 | SeqID 113 | SeqID 34 |
| Construct 128 | SeqID 42 | SeqID 1 | SeqID 116 | SeqID 34 |
| Construct 129 | SeqID 42 | SeqID 1 | SeqID 119 | SeqID 34 |
| Construct 130 | SeqID 42 | SeqID 1 | SeqID 120 | SeqID 34 |
| Construct 131 | SeqID 42 | SeqID 1 | SeqID 121 | SeqID 34 |
| Construct 132 | SeqID 42 | SeqID 1 | SeqID 122 | SeqID 34 |
| Construct 133 | SeqID 42 | SeqID 1 | SeqID 123 | SeqID 34 |
| Construct 134 | SeqID 42 | SeqID 1 | SeqID 124 | SeqID 34 |
| Construct 135 | SeqID 42 | SeqID 1 | SeqID 125 | SeqID 34 |
| Construct 136 | SeqID 126 | SeqID 134 | SeqID 120 | SeqID 36 |
| Construct 137 | SeqID 42 | SeqID 134 | SeqID 120 | SeqID 36 |
| Construct 138 | SeqID 127 | SeqID 134 | SeqID 120 | SeqID 36 |
| Construct 139 | SeqID 50 | SeqID 134 | SeqID 120 | SeqID 36 |
| Construct 140 | SeqID 128 | SeqID 134 | SeqID 120 | SeqID 36 |
| Construct 141 | SeqID 52 | SeqID 134 | SeqID 120 | SeqID 36 |
| Construct 142 | SeqID 129 | SeqID 134 | SeqID 120 | SeqID 36 |
| Construct 143 | SeqID 54 | SeqID 134 | SeqID 120 | SeqID 36 |
| Construct 144 | SeqID 130 | SeqID 134 | SeqID 120 | SeqID 36 |
| Construct 145 | SeqID 46 | SeqID 134 | SeqID 120 | SeqID 36 |
| Construct 146 | SeqID 131 | SeqID 134 | SeqID 120 | SeqID 36 |
| Construct 147 | SeqID 47 | SeqID 134 | SeqID 120 | SeqID 36 |
| Construct 148 | SeqID 132 | SeqID 134 | SeqID 120 | SeqID 36 |
| Construct 149 | SeqID 71 | SeqID 134 | SeqID 120 | SeqID 36 |
| Construct 150 | SeqID 133 | SeqID 134 | SeqID 120 | SeqID 36 |
| Construct 151 | SeqID 53 | SeqID 134 | SeqID 120 | SeqID 36 |
| Construct 152 | SeqID 126 | SeqID 135 | SeqID 120 | SeqID 36 |
| Construct 153 | SeqID 42 | SeqID 135 | SeqID 120 | SeqID 36 |
| Construct 154 | SeqID 127 | SeqID 135 | SeqID 120 | SeqID 36 |
| Construct 155 | SeqID 50 | SeqID 135 | SeqID 120 | SeqID 36 |
| Construct 156 | SeqID 128 | SeqID 135 | SeqID 120 | SeqID 36 |
| Construct 157 | SeqID 52 | SeqID 135 | SeqID 120 | SeqID 36 |
| Construct 158 | SeqID 129 | SeqID 135 | SeqID 120 | SeqID 36 |
| Construct 159 | SeqID 54 | SeqID 135 | SeqID 120 | SeqID 36 |
| Construct 160 | SeqID 130 | SeqID 135 | SeqID 120 | SeqID 36 |
| Construct 161 | SeqID 46 | SeqID 135 | SeqID 120 | SeqID 36 |
| Construct 162 | SeqID 131 | SeqID 135 | SeqID 120 | SeqID 36 |
| Construct 163 | SeqID 47 | SeqID 135 | SeqID 120 | SeqID 36 |
| Construct 164 | SeqID 132 | SeqID 135 | SeqID 120 | SeqID 36 |
| Construct 165 | SeqID 71 | SeqID 135 | SeqID 120 | SeqID 36 |
| Construct 166 | SeqID 133 | SeqID 135 | SeqID 120 | SeqID 36 |
| Construct 167 | SeqID 53 | SeqID 135 | SeqID 120 | SeqID 36 |
| Construct 168 | SeqID 138 | SeqID 137 | SeqID 120 | SeqID 36 |
| Construct 169 | SeqID 139 | SeqID 137 | SeqID 120 | SeqID 36 |
| Construct 170 | SeqID 140 | SeqID 137 | SeqID 120 | SeqID 36 |
| Construct 171 | SeqID 141 | SeqID 137 | SeqID 120 | SeqID 36 |
| Construct 172 | SeqID 142 | SeqID 137 | SeqID 120 | SeqID 36 |
| Construct 173 | SeqID 143 | SeqID 137 | SeqID 120 | SeqID 36 |
| Construct 174 | SeqID 144 | SeqID 137 | SeqID 120 | SeqID 36 |
| Construct 175 | SeqID 145 | SeqID 137 | SeqID 120 | SeqID 36 |
| Construct 176 | SeqID 146 | SeqID 137 | SeqID 120 | SeqID 36 |
| Construct 177 | SeqID 128 | SeqID 136 | SeqID 122 | SeqID 73 |
| Construct 178 | SeqID 138 | SeqID 136 | SeqID 120 | SeqID 73 |
| Construct 179 | SeqID 138 | SeqID 136 | SeqID 122 | SeqID 77 |
| Construct 180 | SeqID 128 | SeqID 136 | SeqID 122 | SeqID 77 |
| Construct 181 | SeqID 54 | SeqID 147 | SeqID 122 | SeqID 87 |
| Construct 182 | SeqID 128 | SeqID 136 | SeqID 122 | SeqID 87 |
| Construct 183 | SeqID 126 | SeqID 136 | SeqID 122 | SeqID 77 |
| Construct 184 | SeqID 129 | SeqID 136 | SeqID 122 | SeqID 77 |
| Construct 185 | SeqID 54 | SeqID 147 | SeqID 122 | SeqID 77 |
| Construct 186 | SeqID 45 | SeqID 147 | SeqID 122 | SeqID 81 |
| Construct 187 | SeqID 45 | SeqID 147 | SeqID 120 | SeqID 81 |
| Construct 188 | SeqID 133 | SeqID 136 | SeqID 122 | SeqID 77 |
| Construct 189 | SeqID 126 | SeqID 136 | SeqID 122 | SeqID 73 |
| Construct 190 | SeqID 138 | SeqID 136 | SeqID 122 | SeqID 73 |
| Construct 191 | SeqID 45 | SeqID 147 | SeqID 122 | SeqID 87 |
| Construct 192 | SeqID 126 | SeqID 136 | SeqID 122 | SeqID 81 |
| Construct 193 | SeqID 138 | SeqID 136 | SeqID 122 | SeqID 81 |
| Construct 194 | SeqID 138 | SeqID 136 | SeqID 122 | SeqID 87 |
| Construct 195 | SeqID 128 | SeqID 136 | SeqID 122 | SeqID 81 |
| Construct 196 | SeqID 54 | SeqID 147 | SeqID 122 | SeqID 81 |
| Construct 197 | SeqID 126 | SeqID 136 | SeqID 120 | SeqID 81 |
| Construct 198 | SeqID 138 | SeqID 136 | SeqID 120 | SeqID 81 |
| Construct 199 | SeqID 129 | SeqID 136 | SeqID 122 | SeqID 73 |
| Construct 200 | SeqID 128 | SeqID 147 | SeqID 122 | SeqID 77 |
| Construct 201 | SeqID 128 | SeqID 147 | SeqID 122 | SeqID 81 |
| Construct 202 | SeqID 128 | SeqID 147 | SeqID 122 | SeqID 73 |
| Construct 203 | SeqID 128 | SeqID 147 | SeqID 122 | SeqID 87 |
| Construct 204 | SeqID 54 | SeqID 147 | SeqID 122 | SeqID 73 |
| Construct 205 | SeqID 133 | SeqID 136 | SeqID 122 | SeqID 87 |
| Construct 206 | SeqID 133 | SeqID 136 | SeqID 122 | SeqID 81 |
| Construct 207 | SeqID 128 | SeqID 136 | SeqID 122 | SeqID 107 |
| Construct 208 | SeqID 129 | SeqID 136 | SeqID 122 | SeqID 109 |
| Construct 209 | SeqID 52 | SeqID 147 | SeqID 122 | SeqID 107 |
| Construct 210 | SeqID 126 | SeqID 136 | SeqID 120 | SeqID 109 |
| Construct 211 | SeqID 128 | SeqID 147 | SeqID 122 | SeqID 109 |
| Construct 212 | SeqID 52 | SeqID 147 | SeqID 122 | SeqID 109 |
| Construct 213 | SeqID 128 | SeqID 147 | SeqID 120 | SeqID 34 |
| Construct 214 | SeqID 128 | SeqID 136 | SeqID 122 | SeqID 109 |
| Construct 215 | SeqID 128 | SeqID 136 | SeqID 120 | SeqID 109 |
| Construct 216 | SeqID 128 | SeqID 147 | SeqID 122 | SeqID 107 |
| Construct 217 | SeqID 52 | SeqID 147 | SeqID 120 | SeqID 109 |
| Construct 218 | SeqID 128 | SeqID 147 | SeqID 120 | SeqID 109 |
| Construct 219 | SeqID 51 | SeqID 147 | SeqID 122 | SeqID 107 |
| Construct 220 | SeqID 52 | SeqID 147 | SeqID 120 | SeqID 34 |
| Construct 221 | SeqID 51 | SeqID 147 | SeqID 120 | SeqID 109 |
| Construct 222 | SeqID 51 | SeqID 147 | SeqID 122 | SeqID 109 |
| Construct 223 | SeqID 126 | SeqID 136 | SeqID 122 | SeqID 109 |
| Construct 224 | SeqID 126 | SeqID 136 | SeqID 122 | SeqID 107 |
| Construct 225 | SeqID 128 | SeqID 136 | SeqID 120 | SeqID 34 |
| Construct 226 | SeqID 126 | SeqID 136 | SeqID 120 | SeqID 34 |
| Construct 227 | SeqID 129 | SeqID 136 | SeqID 120 | SeqID 109 |
| Construct 228 | SeqID 129 | SeqID 136 | SeqID 122 | SeqID 107 |
| Construct 229 | SeqID 129 | SeqID 136 | SeqID 120 | SeqID 34 |
| Construct 230 | SeqID 51 | SeqID 147 | SeqID 120 | SeqID 34 |
| Construct 231 | SeqID 153 | SeqID 148 | | |
| Construct 232 | SeqID 153 | SeqID 149 | | |
| Construct 233 | SeqID 153 | SeqID 150 | SeqID 152 | |
| Construct 234 | SeqID 153 | SeqID 151 | SeqID 152 | |
| Construct 235 | SeqID 154 | SeqID 150 | SeqID 152 | |
| Construct 236 | SeqID 154 | SeqID 151 | SeqID 152 | |
| Construct 237 | SeqID 155 | SeqID 150 | SeqID 152 | |
| Construct 238 | SeqID 155 | SeqID 151 | SeqID 152 | |
| Construct 239 | SeqID 157 | SeqID 149 | | |
| Construct 240 | SeqID 156 | SeqID 149 | | |
| Construct 241 | SeqID 138 | SeqID 149 | SeqID 120 | |
| Construct 242 | SeqID 138 | SeqID 149 | | |
| Construct 243 | SeqID 138 | SeqID 150 | SeqID 152 | |
| Construct 244 | SeqID 138 | SeqID 137 | SeqID 120 | SeqID 73 |

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

All publications, patents, and patent applications cited in this specification are herein incorporated by reference as if each individual publication or patent application were specifically and individually indicated to be incorporated by reference.

SEQUENCE APPENDIX

SEQ ID NO: 1-CBDaS from Cannabis sativa
MKCSTFSFWFVCKIIFFFFSFNIQTSIANPRENFLKCFSQYIPNNATNLKLVYTQNNPLYM
SVLNSTIHNLRFTSDTTPKPLVIVTPSHVSHIQGTILCSKKVGLQIRTRSGGHDSEGMSYIS
QVPFVIVDLRNMRSIKIDVHSQTAWVEAGATLGEVYYWVNEKNENLSLAAGYCPTVCA
GGHFGGGGYGPLMRNYGLAADNIIDAHLVNVHGKVLDRKSMGEDLFWALRGGGAESF
GIIVAWKIRLVAVPKSTMFSVKKIMEIHELVKLVNKWQNIAYKYDKDLLLMTHFITRNIT
DNQGKNKTAIHTYFSSVFLGGVDSLVDLMNKSFPELGIKKTDCRQLSWIDTIIFYSGVVN
YDTDNFNKEILLDRSAGQNGAFKIKLDYVKKPIPESVFVQILEKLYEEDIGAGMYALYPY
GGIMDEISESAIPFPHRAGILYELWYICSWEKQEDNEKHLNWIRNIYNFMTPYVSKNPRL
AYLNYRDLDIGINDPKNPNNYTQARIWGEKYFGKNFDRLVKVKTLVDPNNFFRNEQSIP
PLPRHRH
SEQ ID NO: 2-PEP4 signal sequence from Komagataella pastoris
MIFDGTTMSIAIGLLSTLGIGAEA
SEQ ID NO: 3-N-terminal truncation (Δ1-20) CBDaS from Cannabis sativa
FNIQTSIANPRENFLKCFSQYIPNNATNLKLVYTQNNPLYMSVLNSTIHNLRFTSDTTPKP
LVIVTPSHVSHIQGTILCSKKVGLQIRTRSGGHDSEGMSYISQVPFVIVDLRNMRSIKIDV
HSQTAWVEAGATLGEVYYWVNEKNENLSLAAGYCPTVCAGGHFGGGGYGPLMRNYG
LAADNIIDAHLVNVHGKVLDRKSMGEDLFWALRGGGAESFGIIVAWKIRLVAVPKSTM
FSVKKIMEIHELVKLVNKWQNIAYKYDKDLLLMTHFITRNITDNQGKNKTAIHTYFSSVF
LGGVDSLVDLMNKSFPELGIKKTDCRQLSWIDTIIFYSGVVNYDTDNFNKEILLDRSAGQ
NGAFKIKLDYVKKPIPESVFVQILEKLYEEDIGAGMYALYPYGGIMDEISESAIPFPHRAG
ILYELWYICSWEKQEDNEKHLNWIRNIYNFMTPYVSKNPRLAYLNYRDLDIGINDPKNP
NNYTQARIWGEKYFGKNFDRLVKVKTLVDPNNFFRNEQSIPPLPRHRH
SEQ ID NO: 4-N-terminal truncation (Δ1-21) CBDaS from Cannabis sativa
NIQTSIANPRENFLKCFSQYIPNNATNLKLVYTQNNPLYMSVLNSTIHNLRFTSDTTPKPL
VIVTPSHVSHIQGTILCSKKVGLQIRTRSGGHDSEGMSYISQVPFVIVDLRNMRSIKIDVHS
QTAWVEAGATLGEVYYWVNEKNENLSLAAGYCPTVCAGGHFGGGGYGPLMRNYGLA
ADNIIDAHLVNVHGKVLDRKSMGEDLFWALRGGGAESFGIIVAWKIRLVAVPKSTMFS
VKKIMEIHELVKLVNKWQNIAYKYDKDLLLMTHFITRNITDNQGKNKTAIHTYFSSVFL
GGVDSLVDLMNKSFPELGIKKTDCRQLSWIDTIIFYSGVVNYDTDNFNKEILLDRSAGQN
GAFKIKLDYVKKPIPESVFVQILEKLYEEDIGAGMYALYPYGGIMDEISESAIPFPHRAGIL
YELWYICSWEKQEDNEKHLNWIRNIYNFMTPYVSKNPRLAYLNYRDLDIGINDPKNPNN
YTQARIWGEKYFGKNFDRLVKVKTLVDPNNFFRNEQSIPPLPRHRH
SEQ ID NO: 5-N-terminal truncation (Δ1-22) CBDaS from Cannabis sativa
IQTSIANPRENFLKCFSQYIPNNATNLKLVYTQNNPLYMSVLNSTIHNLRFTSDTTPKPLV
IVTPSHVSHIQGTILCSKKVGLQIRTRSGGHDSEGMSYISQVPFVIVDLRNMRSIKIDVHSQ
TAWVEAGATLGEVYYWVNEKNENLSLAAGYCPTVCAGGHFGGGGYGPLMRNYGLAA
DNIIDAHLVNVHGKVLDRKSMGEDLFWALRGGGAESFGIIVAWKIRLVAVPKSTMFSV
KKIMEIHELVKLVNKWQNIAYKYDKDLLLMTHFITRNITDNQGKNKTAIHTYFSSVFLG
GVDSLVDLMNKSFPELGIKKTDCRQLSWIDTIIFYSGVVNYDTDNFNKEILLDRSAGQNG
AFKIKLDYVKKPIPESVFVQILEKLYEEDIGAGMYALYPYGGIMDEISESAIPFPHRAGILY
ELWYICSWEKQEDNEKHLNWIRNIYNFMTPYVSKNPRLAYLNYRDLDIGINDPKNPNNY
TQARIWGEKYFGKNFDRLVKVKTLVDPNNFFRNEQSIPPLPRHRH
SEQ ID NO: 6-N-terminal truncation (Δ1-23) CBDaS from Cannabis sativa
QTSIANPRENFLKCFSQYIPNNATNLKLVYTQNNPLYMSVLNSTIHNLRFTSDTTPKPLVI
VTPSHVSHIQGTILCSKKVGLQIRTRSGGHDSEGMSYISQVPFVIVDLRNMRSIKIDVHSQ
TAWVEAGATLGEVYYWVNEKNENLSLAAGYCPTVCAGGHFGGGGYGPLMRNYGLAA
DNIIDAHLVNVHGKVLDRKSMGEDLFWALRGGGAESFGIIVAWKIRLVAVPKSTMFSV
KKIMEIHELVKLVNKWQNIAYKYDKDLLLMTHFITRNITDNQGKNKTAIHTYFSSVFLG
GVDSLVDLMNKSFPELGIKKTDCRQLSWIDTIIFYSGVVNYDTDNFNKEILLDRSAGQNG
AFKIKLDYVKKPIPESVFVQILEKLYEEDIGAGMYALYPYGGIMDEISESAIPFPHRAGILY
ELWYICSWEKQEDNEKHLNWIRNIYNFMTPYVSKNPRLAYLNYRDLDIGINDPKNPNNY
TQARIWGEKYFGKNFDRLVKVKTLVDPNNFFRNEQSIPPLPRHRH
SEQ ID NO: 7-N-terminal truncation (Δ1-25) CBDaS from Cannabis sativa
SIANPRENFLKCFSQYIPNNATNLKLVYTQNNPLYMSVLNSTIHNLRFTSDTTPKPLVIVT
PSHVSHIQGTILCSKKVGLQIRTRSGGHDSEGMSYISQVPFVIVDLRNMRSIKIDVHSQTA
WVEAGATLGEVYYWVNEKNENLSLAAGYCPTVCAGGHFGGGGYGPLMRNYGLAADN
IIDAHLVNVHGKVLDRKSMGEDLFWALRGGGAESFGIIVAWKIRLVAVPKSTMFSVKKI
MEIHELVKLVNKWQNIAYKYDKDLLLMTHFITRNITDNQGKNKTAIHTYFSSVFLGGVD
SLVDLMNKSFPELGIKKTDCRQLSWIDTIIFYSGVVNYDTDNFNKEILLDRSAGQNGAFK
IKLDYVKKPIPESVFVQILEKLYEEDIGAGMYALYPYGGIMDEISESAIPFPHRAGILYEL
WYICSWEKQEDNEKHLNWIRNIYNFMTPYVSKNPRLAYLNYRDLDIGINDPKNPNNYT
QARIWGEKYFGKNFDRLVKVKTLVDPNNFFRNEQSIPPLPRHRH
SEQ ID NO: 8-N-terminal truncation (Δ1-26) CBDaS from Cannabis sativa
IANPRENFLKCFSQYIPNNATNLKLVYTQNNPLYMSVLNSTIHNLRFTSDTTPKPLVIVTP
SHVSHIQGTILCSKKVGLQIRTRSGGHDSEGMSYISQVPFVIVDLRNMRSIKIDVHSQTA
WVEAGATLGEVYYWVNEKNENLSLAAGYCPTVCAGGHFGGGGYGPLMRNYGLAADN
IIDAHLVNVHGKVLDRKSMGEDLFWALRGGGAESFGIIVAWKIRLVAVPKSTMFSVKKI
MEIHELVKLVNKWQNIAYKYDKDLLLMTHFITRNITDNQGKNKTAIHTYFSSVFLGGVD
SLVDLMNKSFPELGIKKTDCRQLSWIDTIIFYSGVVNYDTDNFNKEILLDRSAGQNGAFK
IKLDYVKKPIPESVFVQILEKLYEEDIGAGMYALYPYGGIMDEISESAIPFPHRAGILYEL
WYICSWEKQEDNEKHLNWIRNIYNFMTPYVSKNPRLAYLNYRDLDIGINDPKNPNNYT
QARIWGEKYFGKNFDRLVKVKTLVDPNNFFRNEQSIPPLPRHRH
SEQ ID NO: 9-N-terminal truncation (Δ1-27) CBDaS from Cannabis sativa
ANPRENFLKCFSQYIPNNATNLKLVYTQNNPLYMSVLNSTIHNLRFTSDTTPKPLVIVTPS
HVSHIQGTILCSKKVGLQIRTRSGGHDSEGMSYISQVPFVIVDLRNMRSIKIDVHSQTAW
VEAGATLGEVYYWVNEKNENLSLAAGYCPTVCAGGHFGGGGYGPLMRNYGLAADNII
DAHLVNVHGKVLDRKSMGEDLFWALRGGGAESFGIIVAWKIRLVAVPKSTMFSVKKIM
EIHELVKLVNKWQNIAYKYDKDLLLMTHFITRNITDNQGKNKTAIHTYFSSVFLGGVDS
LVDLMNKSFPELGIKKTDCRQLSWIDTIIFYSGVVNYDTDNFNKEILLDRSAGQNGAFKI
KLDYVKKPIPESVFVQILEKLYEEDIGAGMYALYPYGGIMDEISESAIPFPHRAGILYELW
YICSWEKQEDNEKHLNWIRNIYNFMTPYVSKNPRLAYLNYRDLDIGINDPKNPNNYTQA
RIWGEKYFGKNFDRLVKVKTLVDPNNFFRNEQSIPPLPRHRH
SEQ ID NO: 10-N-terminal truncation (Δ1-28) CBDaS from Cannabis sativa
NPRENFLKCFSQYIPNNATNLKLVYTQNNPLYMSVLNSTIHNLRFTSDTTPKPLVIVTPSH
VSHIQGTILCSKKVGLQIRTRSGGHDSEGMSYISQVPFVIVDLRNMRSIKIDVHSQTAWV
EAGATLGEVYYWVNEKNENLSLAAGYCPTVCAGGHFGGGGYGPLMRNYGLAADNIID
AHLVNVHGKVLDRKSMGEDLFWALRGGGAESFGIIVAWKIRLVAVPKSTMFSVKKIME
IHELVKLVNKWQNIAYKYDKDLLLMTHFITRNITDNQGKNKTAIHTYFSSVFLGGVDSL
VDLMNKSFPELGIKKTDCRQLSWIDTIIFYSGVVNYDTDNFNKEILLDRSAGQNGAFKIK
LDYVKKPIPESVFVQILEKLYEEDIGAGMYALYPYGGIMDEISESAIPFPHRAGILYELWY
ICSWEKQEDNEKHLNWIRNIYNFMTPYVSKNPRLAYLNYRDLDIGINDPKNPNNYTQAR
IWGEKYFGKNFDRLVKVKTLVDPNNFFRNEQSIPPLPRHRH
SEQ ID NO: 11-N-terminal truncation (Δ1-29) CBDaS from Cannabis sativa
PRENFLKCFSQYIPNNATNLKLVYTQNNPLYMSVLNSTIHNLRFTSDTTPKPLVIVTPSHV
SHIQGTILCSKKVGLQIRTRSGGHDSEGMSYISQVPFVIVDLRNMRSIKIDVHSQTAWVE
AGATLGEVYYWVNEKNENLSLAAGYCPTVCAGGHFGGGGYGPLMRNYGLAADNIIDA
HLVNVHGKVLDRKSMGEDLFWALRGGGAESFGIIVAWKIRLVAVPKSTMFSVKKIMEI
HELVKLVNKWQNIAYKYDKDLLLMTHFITRNITDNQGKNKTAIHTYFSSVFLGGVDSLV
DLMNKSFPELGIKKTDCRQLSWIDTIIFYSGVVNYDTDNFNKEILLDRSAGQNGAFKIKL
DYVKKPIPESVFVQILEKLYEEDIGAGMYALYPYGGIMDEISESAIPFPHRAGILYELWYI
CSWEKQEDNEKHLNWIRNIYNFMTPYVSKNPRLAYLNYRDLDIGINDPKNPNNYTQARI
WGEKYFGKNFDRLVKVKTLVDPNNFFRNEQSIPPLPRHRH
SEQ ID NO: 12-N-terminal truncation (Δ1-30) CBDaS from Cannabis sativa
RENFLKCFSQYIPNNATNLKLVYTQNNPLYMSVLNSTIHNLRFTSDTTPKPLVIVTPSHVS
HIQGTILCSKKVGLQIRTRSGGHDSEGMSYISQVPFVIVDLRNMRSIKIDVHSQTAWVEA
GATLGEVYYWVNEKNENLSLAAGYCPTVCAGGHFGGGGYGPLMRNYGLAADNIIDAH
LVNVHGKVLDRKSMGEDLFWALRGGGAESFGIIVAWKIRLVAVPKSTMFSVKKIMETHE
LVKLVNKWQNIAYKYDKDLLLMTHFITRNITDNQGKNKTAIHTYFSSVFLGGVDSLVDL
MNKSFPELGIKKTDCRQLSWIDTIIFYSGVVNYDTDNFNKEILLDRSAGQNGAFKIKLDY
VKKPIPESVFVQILEKLYEEDIGAGMYALYPYGGIMDEISESAIPFPHRAGILYELWYICS
WEKQEDNEKHLNWIRNIYNFMTPYVSKNPRLAYLNYRDLDIGINDPKNPNNYTQARIW
GEKYFGKNFDRLVKVKTLVDPNNFFRNEQSIPPLPRHRH
SEQ ID NO: 13-N-terminal truncation (Δ1-31) CBDaS from Cannabis sativa
ENFLKCFSQYIPNNATNLKLVYTQNNPLYMSVLNSTIHNLRFTSDTTPKPLVIVTPSHVSH
IQGTILCSKKVGLQIRTRSGGHDSEGMSYISQVPFVIVDLRNMRSIKIDVHSQTAWVEAG
ATLGEVYYWVNEKNENLSLAAGYCPTVCAGGHFGGGGYGPLMRNYGLAADNIIDAHL
VNVHGKVLDRKSMGEDLFWALRGGGAESFGIIVAWKIRLVAVPKSTMFSVKKIMEIHEL
VKLVNKWQNIAYKYDKDLLLMTHFITRNITDNQGKNKTAIHTYFSSVFLGGVDSLVDL
MNKSFPELGIKKTDCRQLSWIDTIIFYSGVVNYDTDNFNKEILLDRSAGQNGAFKIKLDY
VKKPIPESVFVQILEKLYEEDIGAGMYALYPYGGIMDEISESAIPFPHRAGILYELWYICS
WEKQEDNEKHLNWIRNIYNFMTPYVSKNPRLAYLNYRDLDIGINDPKNPNNYTQARIW
GEKYFGKNFDRLVKVKTLVDPNNFFRNEQSIPPLPRHRH
SEQ ID NO: 14-N-terminal truncation (Δ1-32) CBDaS from Cannabis sativa
NFLKCFSQYIPNNATNLKLVYTQNNPLYMSVLNSTIHNLRFTSDTTPKPLVIVTPSHVSHI
QGTILCSKKVGLQIRTRSGGHDSEGMSYISQVPFVIVDLRNMRSIKIDVHSQTAWVEAGA
TLGEVYYWVNEKNENLSLAAGYCPTVCAGGHFGGGGYGPLMRNYGLAADNIIDAHLV
NVHGKVLDRKSMGEDLFWALRGGGAESFGIIVAWKIRLVAVPKSTMFSVKKIMEIHELV
KLVNKWQNIAYKYDKDLLLMTHFITRNITDNQGKNKTAIHTYFSSVFLGGVDSLVDLM
NKSFPELGIKKTDCRQLSWIDTIIFYSGVVNYDTDNFNKEILLDRSAGQNGAFKIKLDYV
KKPIPESVFVQILEKLYEEDIGAGMYALYPYGGIMDEISESAIPFPHRAGILYELWYICSW
EKQEDNEKHLNWIRNIYNFMTPYVSKNPRLAYLNYRDLDIGINDPKNPNNYTQARIWGE
KYFGKNFDRLVKVKTLVDPNNFFRNEQSIPPLPRHRH
SEQ ID NO: 15-CBDaS natural diversity variant from Cannabis sativa
NPRENFLKCFSQYIPNNATNLKLVYTQNNPLYMSVLNSTIHNLRFTSDTTPKPLVIVTPSH
VSHIQGTILCSKKVGLQIRTRSGGHDSEGMSYISQVPFVIVDLRNMRSIKIDVHSQTAWV
EAGATLGEVYYWVNEKNENLSLAAGYCPTVCAGGHFGGGGYGPLMRNYGLAADNIID
AHLVNVHGKVLDRKSMGEDLFWALRGGGAESFGIIVAWKIRLVAVPKSTMFSVKKIME
IHELVKLVNKWQNIAYKYDKDLLLMTHFITRNITDNQGKNKTAIHTYFSSVFLGGVDSL
VDLMNKSFPELGIKKTDCRQLSWIDTIIFYSGVVNYDTDNFNKEILLDRSAGQNGAFKIK
LDYVKKPIPESVFVQILEKLYEEDIGAGMYALYPYGGIMDEISESAIPFPHRAGILYELWY
ICSWEKQEDNEKHLNWIRNIYNFMTPYVSKNPRLAYLNYRDLDIGINDPKNPNNYTQAR
IWGEKYFGKNFDRLVKVKTLVDPNNFFRNEQSIPPQPRHRH*
SEQ ID NO: 16-CBDaS natural diversity variant from Cannabis sativa
NPRENFLKCFSQYIPNNATNLKLVYTQNNPLYMSVLNSTIHNLRFTSDTTPKPLVIVTPSH
VSHIQGTILCSKKVGLQIRTRSGGHDSEGMSYISQVPFVIVDLRNMRSIKIDVHSQTAWV
EAGATLGEVYYWVNEKNENLSLAAGYCPTVCAGGHFGGGGYGPLMRNYGLAADNIID
AHLVNVHGKVLDRKSMGEDLFWALRGGGAESFGIIVAWKIRLVAVPKSTMFSVKKIME
IHELVKLVNKWQNIAYKYDKDLLLMTHFITRNITDNQGKNKTAIHTYFSSVFLGGVDSL
VDLMNKSFPELGIKKTDCRQLSWIDTIIFYSGVVNYDTDNFNKEILLDRSAGQNGAFKIK
LDYVKKPIPESVFVQILEKLYEEDIGAGMYALYPYGGIMDEISESAIPFPHRAGILYELWY
ICSWEKQEDNEKHLNWIRNIYNFMTPYVSKNSRLAYLNYRDLDIGINDPKNPNNYTQAR
IWGEKYFGKNFDRLVKVKTLVDPNNFFRNEQSIPPLPRHRH*
SEQ ID NO: 17-CBDaS natural diversity variant from Cannabis sativa
NPRENFLKCFSQYIPNNATNLKLVYTQNNPLYMSVLNSTIHNLRFTSDTTPKPLVIVTPSH
VSHIQGTILCSKKVGLQIRTRSGGHDSEGMSYISQVPFVIVDLRNMRSIKIDVRSQTAWV
EAGATLGEVYYWVNEKNENLSLAAGYCPTVCAGGHFGGGGYGPLMRNYGLAADNIID
AHLVNVHGKVLDRKSMGEDLFWALRGGGAESFGIIVAWKIRLVAVPKSTMFSVKKIME
IHELVKLVNKWQNIAYKYDKDLLLMTHFITRNITDNQGKNKTAIHTYFSSVFLGGVDSL
VDLMNKSFPELGIKKTDCRQLSWIDTIIFYSGVVNYDTDNFNKEILLDRSAGQNGAFKIK
LDYVKKPIPESVFVQILEKLYEEDIGAGMYALYPYGGIMDEISESAIPFPHRAGILYELWY
ICSWEKQEDNEKHLNWIRNIYNFMTPYVSKNPRLAYLNYRDLDIGINDPKNPNNYTQAR
IWGEKYFGKNFDRLVKVKTLVDPNNFFRNEQSIPPLPRHRH*
SEQ ID NO: 18-CBDaS natural diversity variant from Cannabis sativa
NPRENFLKCFSQYIPNNATNLKLVYTQNNPLYMSVLNSTIHNLRFTSDTTPKPLVIVTPSH
VSHIQGTILCSKKVGLQIRTRSGGHDSEGMSYISQVPFVIVDLRNMRSIKIDVHSQTAWV
EAGATLGEVYYWVNEKNENLSLAAGYCPTVCAGGHFGGGGYGPLMRNYGLAADNIID
AHLVNVHGKVLDRKSMGEDLFWALRGGGAESFGIIVAWKIRLVAVPKSTMFSVKKIME
IHELVKLVNKWQNIAYKYDKDLLLMTHFITRNITDNQGKNKTAIHTYFSSVFLGGVDSL
VDLMNKSFPELGIKKTDCRQLSWIDTIIFYSGVVNYDTDNFNKEILLDRSAGQNGAFKIK
LDYVKKPIPESVFVQILEKLYEEDIGAGMYALYPYGGIMDEISESAIPFPHRAGILYELWY
ICSWEKQEDNEKHLNWIRNIYNFMTPYVSKNSRLAYLNYRDLDIGINDPKNPNNYTQAR
IWGEKYFGKNFDRLVKVKTLVDPNNFFRNEQSIPPQPRHRH*
SEQ ID NO: 19-CBDaS natural diversity variant from Cannabis sativa
NPRENFLKCFSQYIPNNATNLKLVYTQNNPLYMSVLNSTIHNLRFSSDTTPKPLVIVTPSH
VSHIQGTILCSKKVGLQIRTRSGGHDSEGMSYISQVPFVIVDLRNMRSIKIDVHSQTAWV
EAGATLGEVYYWVNEKNESLSLAAGYCPTVCAGGHFGGGGYGPLMRSYGLAADNIID
AHLVNVHGKVLDRKSMGEDLFWALRGGGAESFGIIVAWKIRLVAVPKSTMFSVKKIME
IHELVKLVNKWQNIAYKYDKDLLLMTHFITRNITDNQGKNKTAIHTYFSSVFLGGVDSL
VDLMNKSFPELGIKKTDCRQLSWIDTIIFYSGVVNYDTDNFNKEILLDRSAGQNGAFKIK
LDYVKKPIPESVFVQILEKLYEEDIGAGMYALYPYGGIMDEISESAIPFPHRAGILYELWY
ICSWEKQEDNEKHLNWIRNIYNFMTPYVSQNPRLAYLNYRDLDIGINDPKNPNNYTQAR
IWGEKYFGKNFDRLVKVKTLVDPNNFFRNEQSIPPLPRHRH*
SEQ ID NO: 20-CBDaS natural diversity variant from Cannabis sativa
NPRENFLKCFSQYIPNNATNLKLVYTQNNPLYMSVLNSTIHNLRFSSDTTPKPLVIVTPSH
VSHIQGTILCSKKVGLQIRTRSGGHDSEGMSYISQVPFVIVDLRNMRSIKIDVHSQTAWV
EAGATLGEVYYWVNEKNESLSLAAGYCPTVCAGGHFGGGGYGPLMRSYGLAADNIID
AHLVNVHGKVLDRKSMGEDLFWALRGGGAESFGIIVAWKIRLVAVPKSTMFSVKKIME
IHELVKLVNKWQNIAYKYDKDLLLMTHFITRNITDNQGKNKTAIHTYFSSVFLGGVDSL
VDLMNKSFPELGIKKTDCRQLSWIDTIIFYSGVVNYDTDNFNKEILLDRSAGQNGAFKIK
LDYVKKPIPESVFVQILEKLYEEDIGAGMYALYPYGGIMDEISESAIPFPHRAGILYELWY
ICSWEKQEDNEKHLNWIRNIYNFMTPYVSQNPRLAYLNYRDLDIRINDPKNPNNYTQAR
IWGEKYFGKNFDRLVKVKTLVDPNNFFRNEQSIPPLPRHRH*
SEQ ID NO: 21-CBDaS natural diversity variant from Cannabis sativa
NPRENFLKCFSQYIPNNATNLKLVYTQNNPLYMSVLNSTIHNLRFSSDTTPKPLVIVTPSH
VSHIQGTILCSKKVGLQIRTRSGGHDSEGMSYISQVPFVIVDLRNMRSIKIDVHSQTAWV
EAGATLGEVYYWVNEKNESLSLAAGYCPTVCAGGHFGGGGYGPLMRSYGLAADNIID
AHLVNVHGKVLDRKSMGEDLFWALRGGGAESFGIIVAWKIRLVAVPKSTMFSVKKIME
IHELVKLVNKWQNIAYKYDKDLLLMTHFITRNITDNQGKNKTAIHTYFSSVFLGGVDSL
VDLMNKSFPELGIKKTDCRQLSWIDTIIFYSGVVNYDTDNFNKEILLDRSARQNGAFKIK
LDYVKKPIPESVFVQILEKLYEEDIGAGMYALYPYGGIMDEISESAIPFPHRAGILYELWY
ICSWEKQEDNEKHLNWIRNIYNFMTPYVSQNPRLAYLNYRDLDIGINDPKNPNNYTQAR
IWGEKYFGKNFDRLVKVKTLVDPNNFFRNEQSIPPLPRHRH*
SEQ ID NO: 22-CBDaS natural diversity variant from Cannabis sativa
NPRENFLKCFSQYIPNNATNLKLVYTQNNPLYMSVLNSTIHNLRFTSDTTPKPLVIVTPSH
VSHIQGTILCSKKVGLQIRTRSGGHDSEGMSYISQVPFVIVDLRNMRSIKIDVHSQTAWV
EAGATLGEVYYWVNEKNENLSLAAGYCPTVCAGGHFGGGGYGPLMRNYGLAADNIID
AHLVNVHGKVLDRKSMGEDLFWALRGGGAESFGIIVAWKIRLVAVPKSTMFSVKKIME
IHELVKLVNKWQNIAYKYDKDLLLMTHFITRNITDNQGKNKTAIHTYFSSVFLGGVDSL
VDLMNKSFPELGIKKTDCRQLSWIDTIIFYSGVVNYDTDNFNKEILLDRSAGQNGAFKIK
LDYVKKPIPESVFVQILEKLYEEDIGAGMYALYPYGGIMDEISESAIPFPHRAGILYELWY
ICSWEKQEDNEKHLNWIRNIYNFMTPHVSQNSRLAYINYRDLDIGINDPKNPNNYTQARI
WGEKYFGKNFDRLVKVKTLVDPNNFFRNEQSIPPLPRHRH*
SEQ ID NO: 23-CBDaS natural diversity variant from Cannabis sativa
NPRENFLKCFSQYIPNNATNLKLVYTQNNPLYMSVLNSTIHNLRFSSDTTPKPLVIVTPSH
VSHIQGTILCSKKVGLQIRTRSGGHDSEGMSYISQVPFVIVDLRNMRSIKIDVHSQTAWV
EAGATLGEVYYWVNEKNESLSLAAGYCPTVCAGGHFGGGGYGPLMRSYGLAADNIID
AHLVNVHGKVLDRKSMGEDLFWALRGGGAESFGIIVAWKIRLVAVPKSTMFSVKKIME
IHELVKLVNKWQNIAYKYDKDLLLMTHFITRNITDNQGKNKTAIHTYFSSVFLGGVDSL
VDLMNKSFPELGIKKTDCRQLSWIDTIIFYSGVVNYDTDNFNKEILLDRSAGQNGAFKIK
LDYVKKPIPESVFVQILEKLYEEDIGAGMYALYPYGGIMDEISESAIPFPHRAGILYELWY
ICSWEKQEDNEKHLNWIRNIYNFMTPYVSQNPRLAYLNYRDLDIGINDPKHPNNPTHARI
RAQKYFRQNFDKLVKVKTLVDPNNFFRNEQSIPPLPRHRH*
SEQ ID NO: 24-FLO1 carrier protein from Saccharomyces cerevisiae
MTMPHRYMFLAVETLLALTSVASGATEACLPAGQRKSGMNINFYQYSLKDSSTYSNAA
YMAYGYASKTKLGSVGGQTDISIDYNIPCVSSSGTFPCPQEDSYGNWGCKGMGACSNS
QGIAYWSTDLFGFYTTPTNVTLEMTGYFLPPQTGSYTFKFATVDDSAILSVGGATAFNC
CAQQQPPITSTNFTIDGIKPWGGSLPPNIEGTVYMYAGYYYPMKVVYSNAVSWGTLPIS
VTLPDGTTVSDDFEGYVYSFDDDLSQSNCTVPDPSNYAVSTTTTTTEPWTGTFTSTSTEM
TTVTGTNGVPTDETVIVIRTPTTASTIITTTEPWNSTFTSTSTELTTVTGTNGVRTDETIIVI
RTPTTATTAITTTEPWNSTFTSTSTELTTVTGTNGLPTDETIIVIRTPTTATTAMTTTQPWN
DTFTSTSTELTTVTGTNGLPTDETIIVIRTPTTATTAMTTTQPWNDTFTSTSTELTTVTGTN
GLPTDETIIVIRTPTTATTAMTTTQPWNDTFTSTSTEITTVTGTNGLPTDETIIVIRTPTTAT
TAMTTPQPWNDTFTSTSTEMTTVTGTNGLPTDETIIVIRTPTTATTAITTTEPWNSTFTSTS
TEMTTVTGTNGLPTDETIIVIRTPTTATTAITTTQPWNDTFTSTSTEMTTVTGTNGLPTDE
TIIVIRTPTTATTAMTTTQPWNDTFTSTSTEITTVTGTTGLPTDETIIVIRTPTTATTAMTTT
QPWNDTFTSTSTEMTTVTGTNGVPTDETVIVIRTPTSEGLISTTTEPWTGTFTSTSTEMTT
VTGTNGQPTDETVIVIRTPTSEGLVTTTTEPWTGTFTSTSTEMTTITGTNGVPTDETVIVIR
TPTSEGLISTTTEPWTGTFTSTSTEMTTITGTNGQPTDETVIVIRTPTSEGLISTTTEPWTGT
FTSTSTEMTHVTGTNGVPTDETVIVIRTPTSEGLISTTTEPWTGIFTSTSTEVTTITGTNGQ
PTDETVIVIRTPTSEGLISTTTEPWTGTFTS
SEQ ID NO: 25-PIR1 carrier protein from Saccharomyces cerevisiae
MQYKKSLVASALVATSLAAYAPKDPWSTLTPSATYKGGITDYSSTFGIAVEPIATTASSK
AKRAAAISQIGDGQIQATTKTTAAAVSQIGDGQIQATTKTKAAAVSQIGDGQIQATTKTT
SAKTTAAAVSQIGDGQIQATTKTKAAAVSQIGDGQIQATTKTTAAAVSQIGDGQIQATT
KTTAAAVSQIGDGQIQATTNTTVAPVSQITDGQIQATTLTSATIIPSPAPAPITNGTDPVTA
ETCKSSGTLEMNLKGGILTDGKGRIGSIVANRQFQFDGPPPQAGAIYAAGWSITPEGNLA
IGDQDTFYQCLSGNFYNLYDEHIGTQCNAVHLQAIDLLNC
SEQ ID NO: 26-PIR2 carrier protein from Saccharomyces cerevisiae
MQYKKTLVASALAATTLAAYAPSEPWSTLTPTATYSGGVTDYASTFGIAVQPISTTSSAS
SAATTASSKAKRAASQIGDGQVQAATTTASVSTKSTAAAVSQIGDGQIQATTKTTAAAV
SQIGDGQIQATTKTTSAKTTAAAVSQISDGQIQATTTTLAPKSTAAAVSQIGDGQVQATT
TTLAPKSTAAAVSQIGDGQVQATTKTTAAAVSQIGDGQVQATTKTTAAAVSQIGDGQV
QATTKTTAAAVSQIGDGQVQATTKTTAAAVSQITDGQVQATTKTTQAASQVSDGQVQA
TTATSASAAATSTDPVDAVSCKTSGTLEMNLKGGILTDGKGRIGSIVANRQFQFDGPPPQ
AGAIYAAGWSITPDGNLAIGDNDVFYQCLSGTFYNLYDEHIGSQCTPVHLEAIDLIDC
SEQ ID NO: 27-PIR3 carrier protein from Saccharomyces cerevisiae
MQYKKPLVVSALAATSLAAYAPKDPWSTLTPSATYKGGITDYSSSFGIAIEAVATSASSV
ASSKAKRAASQIGDGQVQAATTTAAVSKKSTAAAVSQITDGQVQAAKSTAAAVSQITD
GQVQAAKSTAAAVSQITDGQVQAAKSTAAAVSQITDGQVQAAKSTAAAASQISDGQVQ
ATTSTKAAASQITDGQIQASKTTSGASQVSDGQVQATAEVKDANDPVDVVSCNNNSTL
SMSLSKGILTDRKGRIGSIVANRQFQFDGPPPQAGAIYAAGWSITPEGNLALGDQDTFYQ
CLSGDFYNLYDKHIGSQCHEVYLQAIDLIDC
SEQ ID NO: 28-PIR4 carrier protein from Saccharomyces cerevisiae
MQFKNVALAASVAALSATASAEGYTPGEPWSTLTPTGSISCGAAEYTTTFGIAVQAITSS
KAKRDVISQIGDGQVQATSAATAQATDSQAQATTTATPTSSEKISSSASKTSTNATSSSC
ATPSLKDSSCKNSGTLELTLKDGVLTDAKGRIGSIVANRQFQFDGPPPQAGAIYAAGWSI
TEDGYLALGDSDVFYQCLSGNFYNLYDQNVAEQCSAIHLEAVSLVDC
SEQ ID NO: 29-AGA1 carrier protein from Saccharomyces cerevisiae
TVVSSSAIEPSSASIISPVTSTLSSTTSSNPTTTSLSSTSTSPSSTSTSPSSTSTSSSSTSTSSSST
STSSSSTSTSPSSTSTSSSLTSTSSSSTSTSQSSTSTSSSSTSTSPSSTSTSSSSTSTSPSSKSTSA
SSTSTSSYSTSTSPSLTSSSPTLASTSPSSTSISSTFTDSTSSLGSSIASSSTSVSLYSPSTPVYS
VPSTSSNVATPSMTSSTVETTVSSQSSSEYITKSSISTTIPSFSMSTYFTTVSGVTTMYTTW
CPYSSESETSTLTSMHETVTTDATVCTHESCMPSQTTSLITSSIKMSTKNVATSVSTSTVE
SSYACSTCAETSHSYSSVQTASSSSVTQQTTSTKSWVSSMTTSDEDENKHATGKYHVTS
SGTSTISTSVSEATSTSSIDSESQEQSSHLLSTSVLSSSSLSATLSSDSTILLFSSVSSLSVEQS
PVTTLQISSTSEILQPTSSTAIATISASTSSLSATSISTPSTSVESTIESSSLTPTVSSIFLSSSSA
PSSLQTSVTTTEVSTTSISIQYQTSSMVTISQYMGSGSQTRLPLGKLVFAIMAVACNVIFS
SEQ ID NO: 30-CCW12 carrier protein from Saccharomyces cerevisiae
VDDVITQYTTWCPLTTEAPKNGTSTAAPVTSTEAPKNTTSAAPTHSVTSYTGAAAKALP
AAGALLAGAAALLL
SEQ ID NO: 31-CWP1 carrier protein from Saccharomyces cerevisiae
LVSIRSGSDLQYLSVYSDNGTLKLGSGSGSFEATITDDGKLKFDDDKYAVVNEDGSFKE
GSESDAATGFSIKDGHLNYKSSSGFYAIKDGSSYIFSSKQSDDATGVAIRPTSKSGSVAAD
FSPSDSSSSSSASASSASASSSTKHSSSIESVETSTTVETSSASSPTASVISQITDGQIQAPNT
VYEQTENAGAKAAVGMGAGALAVAAAYLL
SEQ ID NO: 32-CWP2 carrier protein from Saccharomyces cerevisiae
ISQITDGQIQATTTATTEATTTAAPSSTVETVSPSSTETISQQTENGAAKAAVGMGAGAL
AAAAMLL
SEQ ID NO: 33-DAN4 carrier protein from Saccharomyces cerevisiae
SVASFASSSPLLVSSRSNCSDARSSNTISSGLFSTIENVRNATSTFTNLSTDEIVITSCKSSCT
NEDSVLTKTQVSTVETTITSCSGGICTTLMSPVTTINAKANTLTTTETSTVETTITTCPGGV
CSTLTVPVTTITSEATTTATISCEDNEEDITSTETELLTLETTITSCSGGICTTLMSPVTTINA
KANTLTTTETSTVETTITTCSGGVCSTLTVPVTTITSEATTTATISCEDNEEDVASTKTELL
TMETTITSCSGGICTTLMSPVSSFNSKATTSNNAESTIPKAIKVSCSAGACTTLTTVDAGIS
MFTRTGLSITQTTVTNCSGGTCTMLTAPIATATSKVISPIPKASSATSIAHSSASYTVSINT
NGAYNFDKDNIFGTAIVAVVALLLL
SEQ ID NO: 34-FLO5 carrier protein from Saccharomyces cerevisiae
FYPSNGTSVISSSVISSSVTSSLVTSSSFISSSVISSSTTTSTSIFSESSTSSVIPTSSSTSGSSES
KTSSASSSSSSSSISSESPKSPTNSSSSLPPVTSATTGQETASSLPPATTTKTSEQTTLVTVTS
CESHVCTESISSAIVSTATVTVSGVTTEYTTWCPISTTETTKQTKGTTEQTKGTTEQTTET
TKQTTVVTISSCESDICSKTASPAIVSTSTATINGVTTEYTTWCPISTTESKQQTTLVTVTS
CESGVCSETTSPAIVSTATATVNDVVTVYPTWRPQTTNEQSVSSKMNSATSETTTNTGA
AETKTAVTSSLSRFNHAETQTASATDVIGHNNSVVSVSETGNTKSLTSSGLSTMSQQPRS
TPASSMVGYSTASLEISTYAGSANSLLAGSGLSVFIASLLLAII
SEQ ID NO: 35-PRY3 carrier protein from Saccharomyces cerevisiae
SSTSLGARTTTGSNGRSTTSQQDGSAMHQPTSSIYTQLKEGTSTTAKLSAYEGAATPLSIF
QCNSLAGTIAAFVVAVLFAF
SEQ ID NO: 36-SAG1 carrier protein from Saccharomyces cerevisiae
SAKSSFISTTTTDLTSINTSAYSTGSISTVETGNRTTSEVISHVVTTSTKLSPTATTSLTIAQT
SIYSTDSNITVGTDIHTTSEVISDVETISRETASTVVAAPTSTTGWTGAMNTYISQFTSSSF
ATINSTPIISSSAVFETSDASIVNVHTENITNTAAVPSEEPTFVNATRNSLNSFCSSKQPSSP
SSYTSSPLVSSLSVSKTLLSTSFTPSVPTSNTYIKTKNTGYFEHTALTTSSVGLNSFSETAV
SSQGTKIDTFLVSSLIAYPSSASGSQLSGIQQNFTSTSLMISTYEGKASIFFSAELGSIIFLLL
SYLLF
SEQ ID NO: 37-SED1 carrier protein from Saccharomyces cerevisiae
ALPTNGTSTEAPTDTTTEAPTTGLPTNGTTSAFPPTTSLPPSNTTTTPPYNPSTDYTTDYTV
VTEYTTYCPEPTTFTTNGKTYTVTEPTTLTITDCPCTIEKPTTTSTTEYTVVTEYTTYCPEP
TTFTTNGKTYTVTEPTTLTITDCPCTIEKSEAPESSVPVTESKGTTTKETGVTTKQTTANPS
LTVSTVVPVSSSASSHSVVINSNGANVVVPGALGLAGVAMLFL
SEQ ID NO: 38-SRP2 carrier protein from Saccharomyces cerevisiae
SSEASSSAATSSAVASSSEATSSTVASSTKAASSTKASSSAVSSAVASSTKASAISQISDGQ
VQATSTVSEQTENGAAKAVIGMGAGVMAAAAMLL
SEQ ID NO: 39-TIP1 carrier protein from Saccharomyces cerevisiae
MTYTDDAYTTLFSELDFDAITKTIVKLPWYTTRLSSEIAAALASVSPASSEAASSSEAASS
SKAASSSEATSSAAPSSSAAPSSSAAPSSSAESSSKAVSSSVAPTTSSVSTSTVETASNAGQ
RVNAGAASFGAVVAGAAALLL
SEQ ID NO: 40-TIR1 carrier protein from Saccharomyces cerevisiae
SLASDSSSGFSLSSMPAGVLDIGMALASATDDSYTTLYSEVDFAGVSKMLTMVPWYSSR
LEPALKSLNGDASSSAAPSSSAAPTSSAAPSSSAAPTSSAASSSSEAKSSSAAPSSSEAKSS
SAAPSSSEAKSSSAAPSSSEAKSSSAAPSSTEAKITSAAPSSTGAKTSAISQITDGQIQATKA
VSEQTENGAAKAFVGMGAGVVAAAAMLL
SEQ ID NO: 41-TOS6 carrier protein from Saccharomyces cerevisiae
TSMVSTVKTTSTPYTTSTIATLSTKSISSQANTTTHEISTYVGAAVKGSVAGMGAIMGAA
AFALL
SEQ ID NO: 42-Signal sequence from Saccharomyces cerevisiae
MQLLRCFSIFSVIASVLA
SEQ ID NO: 43-Signal sequence from Saccharomyces cerevisiae
MTLSFAHFTYLFTILLGLTNIALA
SEQ ID NO: 44-Signal sequence from Saccharomyces cerevisiae
MQFSTVASVAFVALANFVAA
SEQ ID NO: 45-Signal sequence from Saccharomyces cerevisiae
MQFSTVASIAAVAAVASA
SEQ ID NO: 46-Signal sequence from Saccharomyces cerevisiae
MQYKKSLVASALVATSLA
SEQ ID NO: 47-Signal sequence from Saccharomyces cerevisiae
MQYKKPLVVSALAATSLA
SEQ ID NO: 48-Signal sequence from Saccharomyces cerevisiae
MAYIKIALLAAIAALASA
SEQ ID NO: 49-Signal sequence from Saccharomyces cerevisiae
MESVSSLFNIFSTIMVNYKSLVLALLSVSNLKYARG
SEQ ID NO: 50-Signal sequence from Saccharomyces cerevisiae
MSAINHLCLKLILASFAIINTITA
SEQ ID NO: 51-Signal sequence from Saccharomyces cerevisiae
MVNISIVAGIVALATSAAA
SEQ ID NO: 52-Signal sequence from Saccharomyces cerevisiae
MRQVWFSWIVGLFLCFFNVSSA
SEQ ID NO: 53-Signal sequence from Saccharomyces cerevisiae
MLLQAFLFLLAGFAAKISA
SEQ ID NO: 54-Signal sequence from Saccharomyces cerevisiae
MFSLKALLPLALLLVSANQVAA
SEQ ID NO: 55-Signal sequence from Saccharomyces cerevisiae
MKFSTALSVALFALAKMVIA
SEQ ID NO: 56-Acyl-activating enzyme from Cannabis sativa
MGKNYKSLDSVVASDFIALGITSEVAETLHGRLAEIVCNYGAATPQTWINIANHILSPDL
PFSLHQMLFYGCYKDFGPAPPAWIPDPEKVKSTNLGALLEKRGKEFLGVKYKDPISSFSH
FQEFSVRNPEVYWRTVLMDEMKISFSKDPECILRRDDINNPGGSEWLPGGYLNSAKNCL
NVNSNKKLNDTMIVWRDEGNDDLPLNKLTLDQLRKRVWLVGYALEEMGLEKGCAIAI
DMPMHVDAVVIYLAIVLAGYVVVSIADSFSAPEISTRLRLSKAKAIFTQDHIIRGKKRIPL
YSRVVEAKSPMAIVIPCSGSNIGAELRDGDISWDYFLERAKEFKNCEFTAREQPVDAYTN
ILFSSGTTGEPKAIPWTQATPLKAAADGWSHLDIRKGDVIVWPTNLGWMMGPWLVYAS
LLNGASIALYNGSPLVSGFAKFVQDAKVTMLGVVPSIVRSWKSTNCVSGYDWSTIRCFS
SSGEASNVDEYLWLMGRANYKPVIEMCGGTEIGGAFSAGSFLQAQSLSSFSSQCMGCTL
YILDKNGYPMPKNKPGIGELALGPVMFGASKTLLNGNHHDVYFKGMPTLNGEVLRRHG
DIFELTSNGYYHAHGRADDTMNIGGIKISSIEIERVCNEVDDRVFETTAIGVPPLGGGPEQ
LVIFFVLKDSNDTTIDLNQLRLSFNLGLQKKLNPLFKVTRVVPLSSLPRTATNKIMRRVL
RQQFSHFE
SEQ ID NO: 57-Signal sequence from Saccharomyces cerevisiae
MQYKKTLVASALAATTLA
SEQ ID NO: 58-Signal sequence from Saccharomyces cerevisiae
MQFKNVALAASVAALSATASAEGYTPGEPWSTLTPTGSISCGAAEYTTTFGIAVQAITSS
KAKRDVISQIGDGQVQATSAATAQATDSQAQATTTATPTSSEKISSSASKTSTNATSSSC
ATPSLKDSSCKNSGTLELTLKDGVLTDAKGRIGSIVANRQFQFDGPPPQAGAIYAAGWSI
TEDGYLALGDSDVFYQCLSGNFYNLYDQNVAEQCSAIHLEAVSLVDC
SEQ ID NO: 59-Signal sequence from Saccharomyces cerevisiae
MSVSKIAFVLSAIASLAVA
SEQ ID NO: 60-Signal sequence from Saccharomyces cerevisiae
MKLSTVLLSAGLASTTLA
SEQ ID NO: 61-Signal sequence from Saccharomyces cerevisiae
MAYTKIALFAAIAALASA
SEQ ID NO: 62-Signal sequence from Saccharomyces cerevisiae
MLEFPISVLLGCLVAVKA
SEQ ID NO: 63-Signal sequence from Saccharomyces cerevisiae
MKFSTLSTVAAIAAFASA
SEQ ID NO: 64-Signal sequence from Saccharomyces cerevisiae
MTKPTQVLVRSVSILFFITLLHLVVALNDVAGPAETAPVSLLPR
SEQ ID NO: 65-Signal sequence from Saccharomyces cerevisiae
MSRISILAVAAALVASATA
SEQ ID NO: 66-Signal sequence from Saccharomyces cerevisiae
MRFPSIFTAVLFAASSALA
SEQ ID NO: 67-Signal sequence from Saccharomyces cerevisiae
MKAFTSLLCGLGLSTTLAKA
SEQ ID NO: 68-Signal sequence from Saccharomyces cerevisiae
MFNRFNKLQAALALVLYSQSALG
SEQ ID NO: 69-Signal sequence from Saccharomyces cerevisiae
MRFSNFLTVSALLTGALG
SEQ ID NO: 70-Signal sequence from Saccharomyces cerevisiae
MISANSLLISTLCAFAIA
SEQ ID NO: 71-Signal sequence from Saccharomyces cerevisiae
MFTFLKIILWLFSLALASA
SEQ ID NO: 72-Carrier protein from Saccharomyces cerevisiae
YQGRNLGTASAKSSFISTTTTDLTSINTSAYSTGSISTVETGNRTTSEVISHVVTTSTKLSP
TATTSLTIAQTSIYSTDSNITVGTDIHTTSEVISDVETISRETASTVVAAPTSTTGWTGAMN
TYISQFTSSSFATINSTPIISSSAVFETSDASIVNVHTENITNTAAVPSEEPTFVNATRNSLNS
FCSSKQPSSPSSYTSSPLVSSLSVSKTLLSTSFTPSVPTSNTYIKTKNTGYFEHTALTTSSVG
LNSFSETAVSSQGTKIDTFLVSSLIAYPSSASGSQLSGIQQNFTSTSLMISTYEGKASIFFSA
ELGSIIFLLLSYLLF
SEQ ID NO: 73-Carrier protein from Saccharomyces cerevisiae
ASAKSSFISTTTTDLTSINTSAYSTGSISTVETGNRTTSEVISHVVTTSTKLSPTATTSLTIA
QTSIYSTDSNITVGTDIHTTSEVISDVETISRETASTVVAAPTSTTGWTGAMNTYISQFTSS
SFATINSTPIISSSAVFETSDASIVNVHTENITNTAAVPSEEPTFVNATRNSLNSFCSSKQPS
SPSSYTSSPLVSSLSVSKTLLSTSFTPSVPTSNTYIKTKNTGYFEHTALTTSSVGLNSFSETA
VSSQGTKIDTFLVSSLIAYPSSASGSQLSGIQQNFTSTSLMISTYEGKASIFFSAELGSIIFLL
LSYLLF
SEQ ID NO: 74-Tetraketide synthase from Cannabis sativa
MNHLRAEGPASVLAIGTANPENILLQDEFPDYYFRVTKSEHMTQLKEKFRKICDKSMIR
KRNCFLNEEHLKQNPRLVEHEMQTLDARQDMLVVEVPKLGKDACAKAIKEWGQPKSK
ITHLIFTSASTTDMPGADYHCAKLLGLSPSVKRVMMYQLGCYGGGTVLRIAKDIAENNK
GARVLAVCCDIMACLFRGPSESDLELLVGQAIFGDGAAAVIVGAEPDESVGERPIFELVS
TGQTILPNSEGTIGGHIREAGLIFDLHKDVPMLISNNIEKCLIEAFTPIGISDWNSIFWITHP
GGKAILDKVEEKLHLKSDKFVDSRHVLSEHGNMSSSTVLFVMDELRKRSLEEGKSTTGD
GFEWGVLFGFGPGLTVERVVVRSVPIKY
SEQ ID NO: 75-Carrier protein from Saccharomyces cerevisiae
TTTTDLTSINTSAYSTGSISTVETGNRTTSEVISHVVTTSTKLSPTATTSLTIAQTSIYSTDS
NITVGTDIHTTSEVISDVETISRETASTVVAAPTSTTGWTGAMNTYISQFTSSSFATINSTPI
ISSSAVFETSDASIVNVHTENITNTAAVPSEEPTFVNATRNSLNSFCSSKQPSSPSSYTSSPL
VSSLSVSKTLLSTSFTPSVPTSNTYIKTKNTGYFEHTALTTSSVGLNSFSETAVSSQGTKID
TFLVSSLIAYPSSASGSQLSGIQQNFTSTSLMISTYEGKASIFFSAELGSIIFLLLSYLLF
SEQ ID NO: 76-Carrier protein from Saccharomyces cerevisiae
SAYSTGSISTVETGNRTTSEVISHVVTTSTKLSPTATTSLTIAQTSIYSTDSNITVGTDIHTT
SEVISDVETISRETASTVVAAPTSTTGWTGAMNTYISQFTSSSFATINSTPIISSSAVFETSD
ASIVNVHTENITNTAAVPSEEPTFVNATRNSLNSFCSSKQPSSPSSYTSSPLVSSLSVSKTL
LSTSFTPSVPTSNTYIKTKNTGYFEHTALTTSSVGLNSFSETAVSSQGTKIDTFLVSSLIAY
PSSASGSQLSGIQQNFTSTSLMISTYEGKASIFFSAELGSIIFLLLSYLLF
SEQ ID NO: 77-Carrier protein from Saccharomyces cerevisiae
VETGNRTTSEVISHVVTTSTKLSPTATTSLTIAQTSIYSTDSNITVGTDIHTTSEVISDVETIS
RETASTVVAAPTSTTGWTGAMNTYISQFTSSSFATINSTPIISSSAVFETSDASIVNVHTEN
ITNTAAVPSEEPTFVNATRNSLNSFCSSKQPSSPSSYTSSPLVSSLSVSKTLLSTSFTPSVPT
SNTYIKTKNTGYFEHTALTTSSVGLNSFSETAVSSQGTKIDTFLVSSLIAYPSSASGSQLSG
IQQNFTSTSLMISTYEGKASIFFSAELGSIIFLLLSYLLF
SEQ ID NO: 78-Carrier protein from Saccharomyces cerevisiae
VISHVVTTSTKLSPTATTSLTIAQTSIYSTDSNITVGTDIHTTSEVISDVETISRETASTVVA
APTSTTGWTGAMNTYISQFTSSSFATINSTPIISSSAVFETSDASIVNVHTENITNTAAVPSE
EPTFVNATRNSLNSFCSSKQPSSPSSYTSSPLVSSLSVSKTLLSTSFTPSVPTSNTYIKTKNT
GYFEHTALTTSSVGLNSFSETAVSSQGTKIDTFLVSSLIAYPSSASGSQLSGIQQNFTSTSL
MISTYEGKASIFFSAELGSIIFLLLSYLLF
SEQ ID NO: 79-Carrier protein from Saccharomyces cerevisiae
TATTSLTIAQTSIYSTDSNITVGTDIHTTSEVISDVETISRETASTVVAAPTSTTGWTGAMN
TYISQFTSSSFATINSTPIISSSAVFETSDASIVNVHTENITNTAAVPSEEPTFVNATRNSLNS
FCSSKQPSSPSSYTSSPLVSSLSVSKTLLSTSFTPSVPTSNTYIKTKNTGYFEHTALTTSSVG
LNSFSETAVSSQGTKIDTFLVSSLIAYPSSASGSQLSGIQQNFTSTSLMISTYEGKASIFFSA
ELGSIIFLLLSYLLF
SEQ ID NO: 80-Carrier protein from Saccharomyces cerevisiae
TIAQTSIYSTDSNITVGTDIHTTSEVISDVETISRETASTVVAAPTSTTGWTGAMNTYISQF
TSSSFATINSTPIISSSAVFETSDASIVNVHTENITNTAAVPSEEPTFVNATRNSLNSFCSSK
QPSSPSSYTSSPLVSSLSVSKTLLSTSFTPSVPTSNTYIKTKNTGYFEHTALTTSSVGLNSFS
ETAVSSQGTKIDTFLVSSLIAYPSSASGSQLSGIQQNFTSTSLMISTYEGKASIFFSAELGSII
FLLLSYLLF
SEQ ID NO: 81-Carrier protein from Saccharomyces cerevisiae
DSNITVGTDIHTTSEVISDVETISRETASTVVAAPTSTTGWTGAMNTYISQFTSSSFATINS
TPIISSSAVFETSDASIVNVHTENITNTAAVPSEEPTFVNATRNSLNSFCSSKQPSSPSSYTS
SPLVSSLSVSKTLLSTSFTPSVPTSNTYIKTKNTGYFEHTALTTSSVGLNSFSETAVSSQGT
KIDTFLVSSLIAYPSSASGSQLSGIQQNFTSTSLMISTYEGKASIFFSAELGSIIFLLLSYLLF
SEQ ID NO: 82-Carrier protein from Saccharomyces cerevisiae
HTTSEVISDVETISRETASTVVAAPTSTTGWTGAMNTYISQFTSSSFATINSTPIISSSAVFE
TSDASIVNVHTENITNTAAVPSEEPTFVNATRNSLNSFCSSKQPSSPSSYTSSPLVSSLSVS
KTLLSTSFTPSVPTSNTYIKTKNTGYFEHTALTTSSVGLNSFSETAVSSQGTKIDTFLVSSL
IAYPSSASGSQLSGIQQNFTSTSLMISTYEGKASIFFSAELGSIIFLLLSYLLF
SEQ ID NO: 83-Carrier protein from Saccharomyces cerevisiae
ETISRETASTVVAAPTSTTGWTGAMNTYISQFTSSSFATINSTPIISSSAVFETSDASIVNVH
TENITNTAAVPSEEPTFVNATRNSLNSFCSSKQPSSPSSYTSSPLVSSLSVSKILLSTSFTPS
VPTSNTYIKTKNTGYFEHTALTTSSVGLNSFSETAVSSQGTKIDTFLVSSLIAYPSSASGSQ
LSGIQQNFTSTSLMISTYEGKASIFFSAELGSIIFLLLSYLLF
SEQ ID NO: 84-Carrier protein from Saccharomyces cerevisiae
VVAAPTSTTGWTGAMNTYISQFTSSSFATINSTPIISSSAVFETSDASIVNVHTENITNTAA
VPSEEPTFVNATRNSLNSFCSSKQPSSPSSYTSSPLVSSLSVSKTLLSTSFTPSVPTSNTYIK
TKNTGYFEHTALTTSSVGLNSFSETAVSSQGTKIDTFLVSSLIAYPSSASGSQLSGIQQNFT
STSLMISTYEGKASIFFSAELGSIIFLLLSYLLF
SEQ ID NO: 85-Carrier protein from Saccharomyces cerevisiae
WTGAMNTYISQFTSSSFATINSTPIISSSAVFETSDASIVNVHTENITNTAAVPSEEPTFVN
ATRNSLNSFCSSKQPSSPSSYTSSPLVSSLSVSKTLLSTSFTPSVPTSNTYIKTKNTGYFEHT
ALTTSSVGLNSFSETAVSSQGTKIDTFLVSSLIAYPSSASGSQLSGIQQNFTSTSLMISTYE
GKASIFFSAELGSIIFLLLSYLLF
SEQ ID NO: 86-Carrier protein from Saccharomyces cerevisiae
QFTSSSFATINSTPIISSSAVFETSDASIVNVHTENITNTAAVPSEEPTFVNATRNSLNSFCSS
KQPSSPSSYTSSPLVSSLSVSKTLLSTSFTPSVPTSNTYIKTKNTGYFEHTALTTSSVGLNSF
SETAVSSQGTKIDTFLVSSLIAYPSSASGSQLSGIQQNFTSTSLMISTYEGKASIFFSAELGS
IIFLLLSYLLF
SEQ ID NO: 87-Carrier protein from Saccharomyces cerevisiae
NSTPIISSSAVFETSDASIVNVHTENITNTAAVPSEEPTFVNATRNSLNSFCSSKQPSSPSSY
TSSPLVSSLSVSKTLLSTSFTPSVPTSNTYIKTKNTGYFEHTALTTSSVGLNSFSETAVSSQ
GTKIDTFLVSSLIAYPSSASGSQLSGIQQNFTSTSLMISTYEGKASIFFSAELGSIIFLLLSYL
LF
SEQ ID NO: 88-Carrier protein from Saccharomyces cerevisiae
VFETSDASIVNVHTENITNTAAVPSEEPTFVNATRNSLNSFCSSKQPSSPSSYTSSPLVSSL
SVSKTLLSTSFTPSVPTSNTYIKTKNTGYFEHTALTTSSVGLNSFSETAVSSQGTKIDTFLV
SSLIAYPSSASGSQLSGIQQNFTSTSLMISTYEGKASIFFSAELGSIIFLLLSYLLF
SEQ ID NO: 89-Carrier protein from Saccharomyces cerevisiae
NVHTENITNTAAVPSEEPTFVNATRNSLNSFCSSKQPSSPSSYTSSPLVSSLSVSKILLSTS
FTPSVPTSNTYIKTKNTGYFEHTALTTSSVGLNSFSETAVSSQGTKIDTFLVSSLIAYPSSA
SGSQLSGIQQNFTSTSLMISTYEGKASIFFSAELGSIIFLLLSYLLF
SEQ ID NO: 90-Carrier protein from Saccharomyces cerevisiae
AAVPSEEPTFVNATRNSLNSFCSSKQPSSPSSYTSSPLVSSLSVSKTLLSTSFTPSVPTSNTY
IKTKNTGYFEHTALTTSSVGLNSFSETAVSSQGTKIDTFLVSSLIAYPSSASGSQLSGIQQN
FTSTSLMISTYEGKASIFFSAELGSIIFLLLSYLLF
SEQ ID NO: 91-Carrier protein from Saccharomyces cerevisiae
VNATRNSLNSFCSSKQPSSPSSYTSSPLVSSLSVSKTLLSTSFTPSVPTSNTYIKTKNTGYF
EHTALTTSSVGLNSFSETAVSSQGTKIDTFLVSSLIAYPSSASGSQLSGIQQNFTSTSLMIST
YEGKASIFFSAELGSIIFLLLSYLLF
SEQ ID NO: 92-Carrier protein from Saccharomyces cerevisiae
FCSSKQPSSPSSYTSSPLVSSLSVSKTLLSTSFTPSVPTSNTYIKTKNTGYFEHTALTTSSVG
LNSFSETAVSSQGTKIDTFLVSSLIAYPSSASGSQLSGIQQNFTSTSLMISTYEGKASIFFSA
ELGSIIFLLLSYLLF
SEQ ID NO: 93-Carrier protein from Saccharomyces cerevisiae
SSYTSSPLVSSLSVSKTLLSTSFTPSVPTSNTYIKTKNTGYFEHTALTTSSVGLNSFSETAV
SSQGTKIDTFLVSSLIAYPSSASGSQLSGIQQNFTSTSLMISTYEGKASIFFSAELGSIIFLLL
SYLLF
SEQ ID NO: 94-Carrier protein from Saccharomyces cerevisiae
SLSVSKTLLSTSFTPSVPTSNTYIKTKNTGYFEHTALTTSSVGLNSFSETAVSSQGTKIDTF
LVSSLIAYPSSASGSQLSGIQQNFTSTSLMISTYEGKASIFFSAELGSIIFLLLSYLLF
SEQ ID NO: 95-Carrier protein from Saccharomyces cerevisiae
TSFTPSVPTSNTYIKTKNTGYFEHTALTTSSVGLNSFSETAVSSQGTKIDTFLVSSLIAYPS
SASGSQLSGIQQNFTSTSLMISTYEGKASIFFSAELGSIIFLLLSYLLF
SEQ ID NO: 96-Carrier protein from Saccharomyces cerevisiae
NTYIKTKNTGYFEHTALTTSSVGLNSFSETAVSSQGTKIDTFLVSSLIAYPSSASGSQLSGI
QQNFTSTSLMISTYEGKASIFFSAELGSIIFLLLSYLLF
SEQ ID NO: 97-Carrier protein from Saccharomyces cerevisiae
YFEHTALTTSSVGLNSFSETAVSSQGTKIDTFLVSSLIAYPSSASGSQLSGIQQNFTSTSLM
ISTYEGKASIFFSAELGSIIFLLLSYLLF
SEQ ID NO: 98-Carrier protein from Saccharomyces cerevisiae
SVGLNSFSETAVSSQGTKIDTFLVSSLIAYPSSASGSQLSGIQQNFTSTSLMISTYEGKASIF
FSAELGSIIFLLLSYLLF
SEQ ID NO: 99-Carrier protein from Saccharomyces cerevisiae
AVSSQGTKIDTFLVSSLIAYPSSASGSQLSGIQQNFTSTSLMISTYEGKASIFFSAELGSIIFL
LLSYLLF
SEQ ID NO: 100-Carrier protein from Saccharomyces cerevisiae
TFLVSSLIA YPSSASGSQLSGIQQNFTSTSLMISTYEGKASIFFSAELGSIIFLLLSYLLF
SEQ ID NO: 101-Carrier protein from Saccharomyces cerevisiae
PSSASGSQLSGIQQNFTSTSLMISTYEGKASIFFSAELGSIIFLLLSYLLF
SEQ ID NO: 102-Olivetolic acid cyclase from Cannabis sativa
MAVKHLIVLKFKDEITEAQKEEFFKTYVNLVNIIPAMKDVYWGKDVTQKNKEEGYTHI
VEVTFESVETIQDYIIHPAHVGFGDVYRSFWEKLLIFDYTPRK
SEQ ID NO: 103-Carrier protein from Saccharomyces cerevisiae
YPSNGTSVISSSVISSSVTSSLVTSSSFISSSVISSSTTTSTSIFSESSTSSVIPTSSSTSGSSESK
TSSASSSSSSSSISSESPKSPTNSSSSLPPVTSATTGQETASSLPPATTTKTSEQTTLVTVTSC
ESHVCTESISSAIVSTATVTVSGVTTEYTTWCPISTTETTKQTKGTTEQTKGTTEQTTETT
KQTTVVTISSCESDICSKTASPAIVSTSTATINGVTTEYTTWCPISTTESKQQTTLVTVTSC
ESGVCSETTSPAIVSTATATVNDVVTVYPTWRPQTTNEQSVSSKMNSATSETTTNTGAA
ETKTAVTSSLSRFNHAETQTASATDVIGHNNSVVSVSETGNTKSLTSSGLSTMSQQPRST
PASSMVGYSTASLEISTYAGSANSLLAGSGLSVFIASLLLAII
SEQ ID NO: 104-Carrier protein from Saccharomyces cerevisiae
PSNGTSVISSSVISSSVTSSLVTSSSFISSSVISSSTTTSTSIFSESSTSSVIPTSSSTSGSSESKTS
SASSSSSSSSISSESPKSPTNSSSSLPPVTSATTGQETASSLPPATTTKTSEQTTLVTVTSCES
HVCTESISSAIVSTATVTVSGVTTEYTTWCPISTTETTKQTKGTTEQTKGTTEQTTETTKQ
TTVVTISSCESDICSKTASPAIVSTSTATINGVTTEYTTWCPISTTESKQQTTLVTVTSCESG
VCSETTSPAIVSTATATVNDVVTVYPTWRPQTTNEQSVSSKMNSATSETTTNTGAAETK
TAVTSSLSRFNHAETQTASATDVIGHNNSVVSVSETGNTKSLTSSGLSTMSQQPRSTPAS
SMVGYSTASLEISTYAGSANSLLAGSGLSVFIASLLLAII
SEQ ID NO: 105-Carrier protein from Saccharomyces cerevisiae
SNGTSVISSSVISSSVTSSLVTSSSFISSSVISSSTTTSTSIFSESSTSSVIPTSSSTSGSSESKTSS
ASSSSSSSSISSESPKSPTNSSSSLPPVTSATTGQETASSLPPATTTKTSEQTTLVTVTSCESH
VCTESISSAIVSTATVTVSGVTTEYTTWCPISTTETTKQTKGTTEQTKGTTEQTTETTKQT
TVVTISSCESDICSKTASPAIVSTSTATINGVTTEYTTWCPISTTESKQQTTLVTVTSCESG
VCSETTSPAIVSTATATVNDVVTVYPTWRPQTTNEQSVSSKMNSATSETTTNTGAAETK
TAVTSSLSRFNHAETQTASATDVIGHNNSVVSVSETGNTKSLTSSGLSTMSQQPRSTPAS
SMVGYSTASLEISTYAGSANSLLAGSGLSVFIASLLLAII
SEQ ID NO: 106-Carrier protein from Saccharomyces cerevisiae
NGTSVISSSVISSSVTSSLVTSSSFISSSVISSSTTTSTSIFSESSTSSVIPTSSSTSGSSESKTSS
ASSSSSSSSISSESPKSPTNSSSSLPPVTSATTGQETASSLPPATTTKTSEQTTLVTVTSCESH
VCTESISSAIVSTATVTVSGVTTEYTTWCPISTTETTKQTKGTTEQTKGTTEQTTETTKQT
TVVTISSCESDICSKTASPAIVSTSTATINGVTTEYTTWCPISTTESKQQTTLVTVTSCESG
VCSETTSPAIVSTATATVNDVVTVYPTWRPQTTNEQSVSSKMNSATSETTTNTGAAETK
TAVTSSLSRFNHAETQTASATDVIGHNNSVVSVSETGNTKSLTSSGLSTMSQQPRSTPAS
SMVGYSTASLEISTYAGSANSLLAGSGLSVFIASLLLAII
SEQ ID NO: 107-Carrier protein from Saccharomyces cerevisiae
VISSSVTSSLVTSSSFISSSVISSSTTTSTSIFSESSTSSVIPTSSSTSGSSESKTSSASSSSSSSSI
SSESPKSPTNSSSSLPPVTSATTGQETASSLPPATTTKTSEQTTLVTVTSCESHVCTESISSA
IVSTATVTVSGVTTEYTTWCPISTTETTKQTKGTTEQTKGTTEQTTETTKQTTVVTISSCE
SDICSKTASPAIVSTSTATINGVTTEYTTWCPISTTESKQQTTLVTVTSCESGVCSETTSPAI
VSTATATVNDVVTVYPTWRPQTTNEQSVSSKMNSATSETTTNTGAAETKTAVTSSLSRF
NHAETQTASATDVIGHNNSVVSVSETGNTKSLTSSGLSTMSQQPRSTPASSMVGYSTAS
LEISTYAGSANSLLAGSGLSVFIASLLLAII
SEQ ID NO: 108-Carrier protein from Saccharomyces cerevisiae
VTSSSFISSSVISSSTTTSTSIFSESSTSSVIPTSSSTSGSSESKTSSASSSSSSSSISSESPKSPTN
SSSSLPPVTSATTGQETASSLPPATTTKTSEQTTLVTVTSCESHVCTESISSAIVSTATVTVS
GVTTEYTTWCPISTTETTKQTKGTTEQTKGTTEQTTETTKQTTVVTISSCESDICSKTASP
AIVSTSTATINGVTTEYTTWCPISTTESKQQTTLVTVTSCESGVCSETTSPAIVSTATATVN
DVVTVYPTWRPQTTNEQSVSSKMNSATSETTTNTGAAETKTAVTSSLSRFNHAETQTAS
ATDVIGHNNSVVSVSETGNTKSLTSSGLSTMSQQPRSTPASSMVGYSTASLEISTYAGSA
NSLLAGSGLSVFIASLLLAII
SEQ ID NO: 109-Carrier protein from Saccharomyces cerevisiae
VISSSTTTSTSIFSESSTSSVIPTSSSTSGSSESKTSSASSSSSSSSISSESPKSPTNSSSSLPPVT
SATTGQETASSLPPATTTKTSEQTTLVTVTSCESHVCTESISSAIVSTATVTVSGVTTEYTT
WCPISTTETTKQTKGTTEQTKGTTEQTTETTKQTTVVTISSCESDICSKTASPAIVSTSTAT
INGVTTEYTTWCPISTTESKQQTTLVTVTSCESGVCSETTSPAIVSTATATVNDVVTVYPT
WRPQTTNEQSVSSKMNSATSETTTNTGAAETKTAVTSSLSRFNHAETQTASATDVIGHN
NSVVSVSETGNTKSLTSSGLSTMSQQPRSTPASSMVGYSTASLEISTYAGSANSLLAGSG
LSVFIASLLLAII
SEQ ID NO: 110-Carrier protein from Saccharomyces cerevisiae
SIFSESSTSSVIPTSSSTSGSSESKTSSASSSSSSSSISSESPKSPTNSSSSLPPVTSATTGQETA
SSLPPATTTKTSEQTTLVTVTSCESHVCTESISSAIVSTATVTVSGVTTEYTTWCPISTTET
TKQTKGTTEQTKGTTEQTTETTKQTTVVTISSCESDICSKTASPAIVSTSTATINGVTTEYT
TWCPISTTESKQQTTLVTVTSCESGVCSETTSPAIVSTATATVNDVVTVYPTWRPQTTNE
QSVSSKMNSATSETTTNTGAAETKTAVTSSLSRFNHAETQTASATDVIGHNNSVVSVSE
TGNTKSLTSSGLSTMSQQPRSTPASSMVGYSTASLEISTYAGSANSLLAGSGLSVFIASLL
LAII
SEQ ID NO: 111-Carrier protein from Saccharomyces cerevisiae
VIPTSSSTSGSSESKTSSASSSSSSSSISSESPKSPTNSSSSLPPVTSATTGQETASSLPPATTT
KTSEQTTLVTVTSCESHVCTESISSAIVSTATVTVSGVTTEYTTWCPISTTETTKQTKGTTE
QTKGTTEQTTETTKQTTVVTISSCESDICSKTASPAIVSTSTATINGVTTEYTTWCPISTTES
KQQTTLVTVTSCESGVCSETTSPAIVSTATATVNDVVTVYPTWRPQTTNEQSVSSKMNS
ATSETTTNTGAAETKTAVTSSLSRFNHAETQTASATDVIGHNNSVVSVSETGNTKSLTSS
GLSTMSQQPRSTPASSMVGYSTASLEISTYAGSANSLLAGSGLSVFIASLLLAII
SEQ ID NO: 112-Carrier protein from Saccharomyces cerevisiae
SSESKTSSASSSSSSSSISSESPKSPTNSSSSLPPVTSATTGQETASSLPPATTTKTSEQTTLV
TVTSCESHVCTESISSAIVSTATVTVSGVTTEYTTWCPISTTETTKQTKGTTEQTKGTTEQ
TTETTKQTTVVTISSCESDICSKTASPAIVSTSTATINGVTTEYTTWCPISTTESKQQTTLVT
VTSCESGVCSETTSPAIVSTATATVNDVVTVYPTWRPQTTNEQSVSSKMNSATSETTTNT
GAAETKTAVTSSLSRFNHAETQTASATDVIGHNNSVVSVSETGNTKSLTSSGLSTMSQQP
RSTPASSMVGYSTASLEISTYAGSANSLLAGSGLSVFIASLLLAII
SEQ ID NO: 113-Linker
GSGGSG
SEQ ID NO: 114-Linker
GSGSGS
SEQ ID NO: 115-Linker
HHHHGSGGSG
SEQ ID NO: 116-Linker
GSGAGGVSGAGG
SEQ ID NO: 117-Linker
GSGGSGGSGGSG
SEQ ID NO: 118-Linker
HHHHHHGSGGSG
SEQ ID NO: 119-Linker
GSGGSGGSGGSGGSGGSG
SEQ ID NO: 120-Linker
AEAAAKEAAAKA
SEQ ID NO: 121-Linker
APAPAPAPAPAPAPA
SEQ ID NO: 122-Linker
EPEPEPEPEPEPEPE
SEQ ID NO: 123-Linker
KPKPKPKPKPKPKP
SEQ ID NO: 124-Linker
AEAAAKEAAAKEAAAKA
SEQ ID NO: 125-Linker
AEAAAKEAAAKEAAAKEAAAKA
SEQ ID NO: 126-Signal sequence from Saccharomyces cerevisiae
MQLLRCFSIFSVIASVLARR
SEQ ID NO: 127-Signal sequence from Saccharomyces cerevisiae
MSAINHLCLKLILASFAIINTITARR
SEQ ID NO: 128-Signal sequence from Saccharomyces cerevisiae
MRQVWFSWIVGLFLCFFNVSSARR
SEQ ID NO: 129-Signal sequence from Saccharomyces cerevisiae
MFSLKALLPLALLLVSANQVAARR
SEQ ID NO: 130-Signal sequence from Saccharomyces cerevisiae
MQYKKSLVASALVATSLARR
SEQ ID NO: 131-Signal sequence from Saccharomyces cerevisiae
MQYKKPLVVSALAATSLARR
SEQ ID NO: 132-Signal sequence from Saccharomyces cerevisiae
MFTFLKIILWLFSLALASARR
SEQ ID NO: 133-Signal sequence from Saccharomyces cerevisiae
MLLQAFLFLLAGFAAKISARR
SEQ ID NO: 134-CBDaS from Cannabis sativa
TFSFWFVCKIIFFFFSFNIQTSIANPRENFLKCFSQYIPNNATNLKLVYTQNNPLYMSVLNS
TIHNLRFSSDTTPKPLVIVTPSHVSHIQGTILCSKKVGLQIRTRSGGHDSEGMSYISQVPFV
IVDLRNMRSIKIDVHSQTAWVEAGATLGEVYYWVNEKNESLSLAAGYCPTVCAGGHFG
GGGYGPLMRSYGLAADNIIDAHLVNVHGKVLDRKSMGEDLFWALRGGGAESFGIIVA
WKIRLVAVPKSTMFSVKKIMEIHELVKLVNKWQNIAYKYDKDLLLMTHFITRNITDNQG
KNKTAIHTYFSSVFLGGVDSLVDLMNKSFPELGIKKTDCRQLSWIDTIIFYSGVVNYDTD
NFNKEILLDRSAGQNGAFKIKLDYVKKPIPESVFVQILEKLYEEDIGAGMYALYPYGGIM
DEISESAIPFPHRAGILYELWYICSWEKQEDNEKHLNWIRNIYNFMTPYVSQNPRLAYLN
YRDLDIGINDPKNPNNYTQARIWGEKYFGKNFDRLVKVKTLVDPNNFFRNEQSIPPLPR
HRH
SEQ ID NO: 135-CBDaS from Cannabis sativa
FSFWFVCKIIFFFFSFNIQTSIANPRENFLKCFSQYIPNNATNLKLVYTQNNPLYMSVLNST
IHNLRFSSDTTPKPLVIVTPSHVSHIQGTILCSKKVGLQIRTRSGGHDSEGMSYISQVPFVI
VDLRNMRSIKIDVHSQTAWVEAGATLGEVYYWVNEKNESLSLAAGYCPTVCAGGHFG
GGGYGPLMRSYGLAADNIIDAHLVNVHGKVLDRKSMGEDLFWALRGGGAESFGIIVA
WKIRLVAVPKSTMFSVKKIMEIHELVKLVNKWQNIAYKYDKDLLLMTHFITRNITDNQG
KNKTAIHTYFSSVFLGGVDSLVDLMNKSFPELGIKKTDCRQLSWIDTIIFYSGVVNYDTD
NFNKEILLDRSAGQNGAFKIKLDYVKKPIPESVFVQILEKLYEEDIGAGMYALYPYGGIM
DEISESAIPFPHRAGILYELWYICSWEKQEDNEKHLNWIRNIYNFMTPYVSQNPRLAYLN
YRDLDIGINDPKNPNNYTQARIWGEKYFGKNFDRLVKVKTLVDPNNFFRNEQSIPPLPR
HRH
SEQ ID NO: 136-CBDaS from Cannabis sativa
MKCSTFSFWFVCKIIFFFFSFNIQTSIANPRENFLKCFSQYIPNNATNLKLVYTQNNPLYM
SVLNSTIHNLRFSSDTTPKPLVIVTPSHVSHIQGTILCSKKVGLQIRTRSGGHDSEGMSYIS
QVPFVIVDLRNMRSIKIDVHSQTAWVEAGATLGEVYYWVNEKNESLSLAAGYCPTVCA
GGHFGGGGYGPLMRSYGLAADNIIDAHLVNVHGKVLDRKSMGEDLFWALRGGGAESF
GIIVAWKIRLVAVPKSTMFSVKKIMEIHELVKLVNKWQNIAYKYDKDLLLMTHFITRNIT
DNQGKNKTAIHTYFSSVFLGGVDSLVDLMNKSFPELGIKKTDCRQLSWIDTIIFYSGVVN
YDTDNFNKEILLDRSAGQNGAFKIKLDYVKKPIPESVFVQILEKLYEEDIGAGMYALYPY
GGIMDEISESAIPFPHRAGILYELWYICSWEKQEDNEKHLNWIRNIYNFMTPYVSQNPRL
AYLNYRDLDIGINDPKNPNNYTQARIWGEKYFGKNFDRLVKVKTLVDPNNFFRNEQSIP
PLPRHRH
SEQ ID NO: 137-CBDaS from Cannabis sativa
MKCSTFSFWFVCKIIFFFFSFNIQTSIANPRENFLKCFSQYIPNNATNLKLVYTQNNPLYM
SVLNSTIHNLRFSSDTTPKPLVIVTPSHVSHIQGTILCSKKVGLQIRTRSGGHDSEGMSYIS
QVPFVIVDLRNMRSIKIDVHSQTAWVEAGATLGEVYYWVNEKNENLTLAAGYCPTVCA
GGHFGGGGYGPLMRSYGLAADNIIDAHLVNVHGKVLDRKSMGEDLFWALRGGGAESF
GIIVAWKIRLVAVPKSTMFSVKKIMEIHELVKLVNKWQNIAYKYDKDLLLMTHFITRNIT
DNQGKNKTAIHTYFSSVFLGGVDSLVDLMNKTFPELGIKKTDCRQLSWIDTIIFYSGVVN
YDTDNFNKEILLDRSAGQNGAFKIKLDYVKKPIPESVFVQILEKLYEEDIGAGMYALYPY
GGIMDEISESAIPFPHRAGILYELWYICSWEKQEDNEKHLNWIRNIYNFMTPYVSQNPRL
AYLNYRDLDIGINDPKNPNNYTQARIWGEKYFGKNFDRLVKVKTLVDPNNFFRNEQSIP
PLPRHRH
SEQ ID NO: 138-Signal sequence from Saccharomyces cerevisiae
MQFSTVASVAFVALANFVAARR
SEQ ID NO: 139-Signal sequence from Saccharomyces cerevisiae
MQFSTVASVAFVALANFVAAKR
SEQ ID NO: 140-Signal sequence from Saccharomyces cerevisiae
MQFSTVASVAFVALANFVAARRK
SEQ ID NO: 141-Signal sequence from Saccharomyces cerevisiae
MQFSTVASVAFVALANFVAARRQ
SEQ ID NO: 142-Signal sequence from Saccharomyces cerevisiae
MQFSTVASVAFVALANFVAARRW
SEQ ID NO: 143-Signal sequence from Saccharomyces cerevisiae
MQFSTVASVAFVALANFVAARRE
SEQ ID NO: 144-Signal sequence from Saccharomyces cerevisiae
MQFSTVASVAFVALANFVAALDKR
SEQ ID NO: 145-Signal sequence from Saccharomyces cerevisiae
MQFSTVASVAFVALANFVAALDKREAEA
SEQ ID NO: 146-Signal sequence from Saccharomyces cerevisiae
MQFSTVASVAFVALANFVAAKREAEA
SEQ ID NO: 147-CBDaS from Cannabis sativa
QTSIANPRENFLKCFSQYIPNNATNLKLVYTQNNPLYMSVLNSTIHNLRFSSDTTPKPLVI
VTPSHVSHIQGTILCSKKVGLQIRTRSGGHDSEGMSYISQVPFVIVDLRNMRSIKIDVHSQ
TAWVEAGATLGEVYYWVNEKNESLSLAAGYCPTVCAGGHFGGGGYGPLMRSYGLAA
DNIIDAHLVNVHGKVLDRKSMGEDLFWALRGGGAESFGIIVAWKIRLVAVPKSTMFSV
KKIMEIHELVKLVNKWQNIAYKYDKDLLLMTHFITRNITDNQGKNKTAIHTYFSSVFLG
GVDSLVDLMNKSFPELGIKKTDCRQLSWIDTIIFYSGVVNYDTDNFNKEILLDRSAGQNG
AFKIKLDYVKKPIPESVFVQILEKLYEEDIGAGMYALYPYGGIMDEISESAIPFPHRAGILY
ELWYICSWEKQEDNEKHLNWIRNIYNFMTPYVSQNPRLAYLNYRDLDIGINDPKNPNNY
TQARIWGEKYFGKNFDRLVKVKTLVDPNNFFRNEQSIPPLPRHRH
SEQ ID NO: 148-CBDaS from Cannabis sativa
MKCSTFSFWFVCKIIFFFFSFNIQTSIANPTENFLKCFSQYIDNNATNDKLVYTQNNPLYM
SVLNSTIHNLRFSSDTTPKPLVIVTPSHVSHIQGTILCSKKVGLQIRTRSGGHDSEGMSYIS
QVPFVIVDLRNMRSIKIDVHSQTAWVEAGATLGEVYYNVNEKNENLTLAAGYCPTVCA
GGHFGGGGYGPLMRSYGLAADNIIDAHLVNVDGKVLDRKSMGEDLFWALRGGGAESF
GIVVAWKIRLVAVPKSTMFSVKKIMEIHELVKLVNKWQNIAYKYDKDLLLMTHFITRNI
TDNQGNNKTAIHTYFSCVFLGGVDSLVDLMNKTFPELGIKKTDCRQLSWIDTIIFYSGVV
NYDTDNFNKEILLDRSAGQNGAFKIKLDYVKKPIPESVFVQILEKLYEEDIGAGMYALYP
YGGIMDEISESAIPFPHRAGILYELWYICSWEKQEDNEKHLNWIRNIYNFMTPYVSQNPR
LAYLNYRDLDIGINDPKNPNNYTQARIWGEKYFGKNFDRLVKVKTLVDPNNFFRNEQSI
PPLPRHRH
SEQ ID NO: 149-CBDaS from Cannabis sativa
NPRENFLKCFSQYIDNNATNDKLVYTQNNPLYMSVLNSTIHNLRFSSDTTPKPLVIVTPS
HVSHIQGTILCSKKVGLQIRTRSGGHDSEGMSYISQVPFVIVDLRNMRSIKIDVHSQTAW
VEAGATLGEVYYNVNEKNENLTLAAGYCPTVCAGGHFGGGGYGPLMRSYGLAADNII
DAHLVNVDGKVLDRKSMGEDLFWALRGGGAESFGIVVAWKIRLVAVPKSTMFSVKKI
MEIHELVKLVNKWQNIAYKYDKDLLLMTHFITRNITDNQGNNKTAIHTYFSCVFLGGVD
SLVDLMNKTFPELGIKKTDCRQLSWIDTIIFYSGVVNYDTDNFNKEILLDRSAGQNGAFK
IKLDYVKKPIPESVFVQILEKLYEEDIGAGMYALYPYGGIMDEISESAIPFPHRAGILYEL
WYICSWEKQEDNEKHLNWIRNIYNFMTPYVSQNPRLAYLNYRDLDIGINDPKNPNNYT
QARIWGEKYFGKNFDRLVKVKTLVDPNNFFRNEQSIPPLPRHRH
SEQ ID NO: 150-CBDaS from Cannabis sativa
MKCSTFSFWFVCKIIFFFFSFNIQTSIANPTENFLKCFSQYIDNNATNDKLVYTQNNPLYM
SVLNSTIHNLRFSSDTTPKPLVIVTPSHVSHIQGTILCSKKVGLQIRTRSGGHDSEGMSYIS
QVPFVIVDLRNMRSIKIDVHSQTAWVEAGATLGEVYYNVNEKNENLTLAAGYCPTVCA
GGHFGGGGYGPLMRSYGLAADNIIDAHLVNVDGKVLDRKSMGEDLFWALRGGGAESF
GIVVAWKIRLVAVPKSTMFSVKKIMEIHELVKLVNKWQNIAYKYDKDLLLMTHFITRNI
TDNQGNNKTAIHTYFSCVFLGGVDSLVDLMNKTFPELGIKKTDCRQLSWIDTIIFYSGVV
NYDTDNFNKEILLDRSAGQNGAFKIKLDYVKKPIPESVFVQILEKLYEEDIGAGMYALYP
YGGIMDEISESAIPFPHRAGILYELWYICSWEKQEDNEKHLNWIRNIYNFMTPYVSQNPR
LAYLNYRDLDIGINDPKNPNNYTQARIWGEKYFGKNFDRLVKVKTLVDPNNFFRNEQSI
PPLPRHR
SEQ ID NO: 151-CBDaS from Cannabis sativa
NPRENFLKCFSQYIDNNATNDKLVYTQNNPLYMSVLNSTIHNLRFSSDTTPKPLVIVTPS
HVSHIQGTILCSKKVGLQIRTRSGGHDSEGMSYISQVPFVIVDLRNMRSIKIDVHSQTAW
VEAGATLGEVYYNVNEKNENLTLAAGYCPTVCAGGHFGGGGYGPLMRSYGLAADNII
DAHLVNVDGKVLDRKSMGEDLFWALRGGGAESFGIVVAWKIRLVAVPKSTMFSVKKI
MEIHELVKLVNKWQNIAYKYDKDLLLMTHFITRNITDNQGNNKTAIHTYFSCVFLGGVD
SLVDLMNKTFPELGIKKTDCRQLSWIDTIIFYSGVVNYDTDNFNKEILLDRSAGQNGAFK
IKLDYVKKPIPESVFVQILEKLYEEDIGAGMYALYPYGGIMDEISESAIPFPHRAGILYEL
WYICSWEKQEDNEKHLNWIRNIYNFMTPYVSQNPRLAYLNYRDLDIGINDPKNPNNYT
QARIWGEKYFGKNFDRLVKVKTLVDPNNFFRNEQSIPPLPRHR
SEQ ID NO: 152-Linker
VVPAIPN
SEQ ID NO: 153-MF(alpha)-prepro from Saccharomyces cerevisiae
MRFPSIFTAVLFAASSALAAPVNTTTEDETAQIPAEAVIGYLDLEGDFDVAVLPFSNSTN
NGLLFINTTIASIAAKEEGVSLDKREAEA
SEQ ID NO: 154-MF(alpha)-pre, synthetic pro1 from Saccharomyces cerevisiae
MRFPSIFTAVLFAASSALAQPIDDTESNTTSVNLMADDTESRFATNTTLALDVVNLISMA
KREEAEAEAEPK
SEQ ID NO: 155-MF(alpha)-pre, synthetic pro2 from Saccharomyces cerevisiae
MRFPSIFTAVLFAASSALAQPIDDTESQTTSVNLMADDTESAFATQTNSGGLDVVGLISM
AKREEGEPK
SEQ ID NO: 156-PEP4 whole protein from Saccharomyces cerevisiae
MFSLKALLPLALLLVSANQVAAKVHKAKIYKHELSDEMKEVTFEQHLAHLGQKYLTQF
EKANPEVVFSREHPFFTEGGHDVPLTNYLNAQYYTDITLGTPPQNFKVILDTGSSNLWVP
SNECGSLACFLHSKYDHEASSSYKANGTEFAIQYGTGSLEGYISQDTLSIGDLTIPKQDFA
EATSEPGLTFAFGKFDGILGLGYDTISVDKVVPPFYNAIQQDLLDEKRFAFYLGDTSKDT
ENGGEATFGGIDESKFKGDITWLPVRRKAYWEVKFEGIGLGDEYAELESHGAAIDTGTS
LITLPSGLAEMINAEIGAKKGWTGQYTLDCNTRDNLPDLIFNFNGYNFTIGPYDYTLEVS
GSCISAITPMDFPEPVGPLAIVGDAFLRKYYSIYDLGNNAVGLAKAIAEAAAKEAAAKA
SEQ ID NO: 157-PEP4-prepro from Saccharomyces cerevisiae
MFSLKALLPLALLLVSANQVAAKVHKAKIYKHELSDEMKEVTFEQHLAHLGQKYLTQF
EKANPEVVFSREHPFFTEAEAAAKEAAAKA
SEQ ID NO: 158-pGAL1
TGGAACTTTCAGTAATACGCTTAACTGCTCATTGCTATATTGAAGTACGGATTAGAA
GCCGCCGAGCGGGCGACAGCCCTCCGACGGAAGACTCTCCTCCGTGCGTCCTGGTCT
TCACCGGTCGCGTTCCTGAAACGCAGATGTGCCTCGCGCCGCACTGCTCCGAACAAT
AAAGATTCTACAATACTAGCTTTTATGGTTATGAAGAGGAAAAATTGGCAGTAACCT
GGCCCCACAAACCTTCAAATCAACGAATCAAATTAACAACCATAGGATAATAATGC
GATTAGTTTTTTAGCCTTATTTCTGGGGTAATTAATCAGCGAAGCGATGATTTTTGAT
CTATTAACAGATATATAAATGCAAAAGCTGCATAACCACTTTAACTAATACTTTCAA
CATTTTCGGTTTGTATTACTTCTTATTCAAATGTCATAAAAGTATCAACAAAAAATTG
TTAATATACCTCTATACTTTAACGTCAAGGAGAAAAAACTATA
SEQ ID NO: 159-pGAL10
CATCGCTTCGCTGATTAATTACCCCAGAAATAAGGCTAAAAAACTAATCGCATTATT
ATCCTATGGTTGTTAATTTGATTCGTTGATTTGAAGGTTTGTGGGGCCAGGTTACTGC
CAATTTTTCCTCTTCATAACCATAAAAGCTAGTATTGTAGAATCTTTATTGTTCGGAG
CAGTGCGGCGCGAGGCACATCTGCGTTTCAGGAACGCGACCGGTGAAGACCAGGAC
GCACGGAGGAGAGTCTTCCGTCGGAGGGCTGTCGCCCGCTCGGCGGCTTCTAATCCG
TACTTCAATATAGCAATGAGCAGTTAAGCGTATTACTGAAAGTTCCAAAGAGAAGG
TTTTTTTAGGCTAAGATAATGGGGCTCTTTACATTTCCACAACATATAAGTAAGATTA
GATATGGATATGTATATGGTGGTATTGCCATGTAATATGATTATTAAACTTCTTTGCG
TCCATCCAAAAAAAAAGTAAGAATTTTTGAAAATTCAATATAA
SEQ ID NO: 160-pGAL2
GGCTTAAGTAGGTTGCAATTTCTTTTTCTATTAGTAGCTAAAAATGGGTCACGTGATC
TATATTCGAAAGGGGCGGTTGCCTCAGGAAGGCACCGGCGGTCTTTCGTCCGTGCGG
AGATATCTGCGCCGTTCAGGGGTCCATGTGCCTTGGACGATATTAAGGCAGAAGGC
AGTATCGGGGCGGATCACTCCGAACCGAGATTAGTTAAGCCCTTCCCATCTCAAGAT
GGGGAGCAAATGGCATTATACTCCTGCTAGAAAGTTAACTGTGCACATATTCTTAAA
TTATACAATGTTCTGGAGAGCTATTGTTTAAAAAACAAACATTTCGCAGGCTAAAAT
GTGGAGATAGGATTAGTTTTGTAGACATATATAAACAATCAGTAATTGGATTGAAAA
TTTGGTGTTGTGAATTGCTCTTCATTATGCACCTTATTCAATTATCATCAAGAATAGC
AATAGTTAAGTAAACACAAGATTAACATAATAAAAAAAATAATTCTTTCATA
SEQ ID NO: 161-pGAL3
TTTTACTATTATCTTCTACGCTGACAGTAATATCAAACAGTGACACATATTAAACAC
AGTGGTTTCTTTGCATAAACACCATCAGCCTCAAGTCGTCAAGTAAAGATTTCGTGT
TCATGCAGATAGATAACAATCTATATGTTGATAATTAGCGTTGCCTCATCAATGCGA
GATCCGTTTAACCGGACCCTAGTGCACTTACCCCACGTTCGGTCCACTGTGTGCCGA
ACATGCTCCTTCACTATTTTAACATGTGGAATTCTTGAAAGAATGAAATCGCCATGC
CAAGCCATCACACGGTCTTTTATGCAATTGATTGACCGCCTGCAACACATAGGCAGT
AAAATTTTTACTGAAACGTATATAATCATCATAAGCGACAAGTGAGGCAACACCTTT
GTTACCACATTGACAACCCCAGGTATTCATACTTCCTATTAGCGGAATCAGGAGTGC
AAAAAGAGAAAATAAAAGTAAAAAGGTAGGGCAACACATAGT
SEQ ID NO: 162-pGAL7
GGACGGTAGCAACAAGAATATAGCACGAGCCGCGAAGTTCATTTCGTTACTTTTGAT
ATCGCTCACAACTATTGCGAAGCGCTTCAGTGAAAAAATCATAAGGAAAAGTTGTA
AATATTATTGGTAGTATTCGTTTGGTAAAGTAGAGGGGGTAATTTTTCCCCTTTATTT
TGTTCATACATTCTTAAATTGCTTTGCCTCTCCTTTTGGAAAGCTATACTTCGGAGCA
CTGTTGAGCGAAGGCTCATTAGATATATTTTCTGTCATTTTCCTTAACCCAAAAATAA
GGGAAAGGGTCCAAAAAGCGCTCGGACAACTGTTGACCGTGATCCGAAGGACTGGC
TATACAGTGTTCACAAAATAGCCAAGCTGAAAATAATGTGTAGCTATGTTCAGTTAG
TTTGGCTAGCAAAGATATAAAAGCAGGTCGGAAATATTTATGGGCATTATTATGCAG
AGCATCAACATGATAAAAAAAAACAGTTGAATATTCCCTCAAAA
SEQ ID NO: 163-pGAL4
GCGACACAGAGATGACAGACGGTGGCGCAGGATCCGGTTTAAACGAGGATCCCTTA
AGTTTAAACAACAACAGCAAGCAGGTGTGCAAGACACTAGAGACTCCTAACATGAT
GTATGCCAATAAAACACAAGAGATAAACAACATTGCATGGAGGCCCCAGAGGGGCG
ATTGGTTTGGGTGCGTGAGCGGCAAGAAGTTTCAAAACGTCCGCGTCCTTTGAGACA
GCATTCGCCCAGTATTTTTTTTATTCTACAAACCTTCTATAATTTCAAAGTATTTACA
TAATTCTGTATCAGTTTAATCACCATAATATCGTTTTCTTTGTTTAGTGCAATTAATTT
TTCCTATTGTTACTTCGGGCCTTTTTCTGTTTTATGAGCTATTTTTTCCGTCATCCTTC
CCCAGATTTTCAGCTTCATCTCCAGATTGTGTCTACGTAATGCACGCCATCATTTTAA
GAGAGGACAGAGAAGCAAGCCTCCTGAAAG
SEQ ID NO: 164-pMAL1
GATGATGGACACTAGTGTGTCGAGAATGTATCAACTATATATAGTCCTAATGCCACA
CAAATATGAAGTGGGGGAAGCCCATTCTTAATCCGGCTCAATTTTGGTGCGTGATCG
CGGCCTATGTTTGCTTCCAGAAAAAGCTTAGAATAATATTTCTCACCTTTGATGGAA
TGCTCGCGAGTGCTCGTTTTGATTACCCCATATGCATTGTTGCAGCATGCAAGCACT
ATTGCAAGCCACGCATGGAAGAAATTTGCAAACACCTATAGCCCCGCGTTGTTGAG
GAGGTGGACTTGGTGTAGGACCATAAAGCTGTGCACTACTATGGTGAGCTCTGTCGT
CTGGTGACCTTCTATCTCAGGCACATCCTCGTTTTTGTGCATGAGGTTCGAGTCACGC
CCACGGCCTATTAATCCGCGAAATAAATGCGAAATCTAAATTATGACGCAAGGCTG
AGAGATTCTGACACGCCGCATTTGCGGGGCAGTAATTATCGGGCAGTTTTCCGGGGT
TCGGGATGGGGTTTGGAGAGAAAGTTCAACACAGACCAAAACAGCTTGGGACCACT
TGGATGGAGGTCCCCGCAGAAGAGCTCTGGCGCGTTGGACAAACATTGACAATCCA
CGGCAAAATTGTCTACAGTTCCGTGTATGCGGATAGGGATATCTTCGGGAGTATCGC
AATAGGATACAGGCACTGTGCAGATTACGCGACATGATAGCTTTGTATGTTCTACAG
ACTCTGCCGTAGCAGTCTAGATATAATATCGGAGTTTTGTAGCGTCGTAAGGAAAAC
TTGGGTTACACAGGTTTCTTGAGAGCCCTTTGACGTTGATTGCTCTGGCTTCCATCCA
GGCCCTCATGTGGTTCAGGTGCCTCCGCAGTGGCTGGCAAGCGTGGGGGTCAATTAC
GTCACTTCTATTCATGTACCCCAGACTCAATTGTTGACAGCAATTTCAGCGAGAATT
AAATTCCACAATCAATTCTCGCTGAAATAATTAGGCCGTGATTTAATTCTCGCTGAA
ACAGAATCCTGTCTGGGGTACAGATAACAATCAAGTAACTATTATGGACGTGCATA
GGAGGTGGAGTCCATGACGCAAAGGGAAATATTCATTTTATCCTCGCGAAGTTGGG
ATGTGTCAAAGCGTCGCGCTCGCTATAGTGATGAGAATGTCTTTAGTAAGCTTAAGC
CATATAAAGACCTTCCGCCTCCATATTTTTTTTTATCCCTCTTGACAATATTAATTCCT
T
SEQ ID NO: 165-pMAL2
AAGGAATTAATATTGTCAAGAGGGATAAAAAAAAATATGGAGGCGGAAGGTCTTTA
TATGGCTTAAGCTTACTAAAGACATTCTCATCACTATAGCGAGCGCGACGCTTTGAC
ACATCCCAACTTCGCGAGGATAAAATGAATATTTCCCTTTGCGTCATGGACTCCACC
TCCTATGCACGTCCATAATAGTTACTTGATTGTTATCTGTACCCCAGACAGGATTCTG
TTTCAGCGAGAATTAAATCACGGCCTAATTATTTCAGCGAGAATTGATTGTGGAATT
TAATTCTCGCTGAAATTGCTGTCAACAATTGAGTCTGGGGTACATGAATAGAAGTGA
CGTAATTGACCCCCACGCTTGCCAGCCACTGCGGAGGCACCTGAACCACATGAGGG
CCTGGATGGAAGCCAGAGCAATCAACGTCAAAGGGCTCTCAAGAAACCTGTGTAAC
CCAAGTTTTCCTTACGACGCTACAAAACTCCGATATTATATCTAGACTGCTACGGCA
GAGTCTGTAGAACATACAAAGCTATCATGTCGCGTAATCTGCACAGTGCCTGTATCC
TATTGCGATACTCCCGAAGATATCCCTATCCGCATACACGGAACTGTAGACAATTTT
GCCGTGGATTGTCAATGTTTGTCCAACGCGCCAGAGCTCTTCTGCGGGGACCTCCAT
CCAAGTGGTCCCAAGCTGTTTTGGTCTGTGTTGAACTTTCTCTCCAAACCCCATCCCG
AACCCCGGAAAACTGCCCGATAATTACTGCCCCGCAAATGCGGCGTGTCAGAATCT
CTCAGCCTTGCGTCATAATTTAGATTTCGCATTTATTTCGCGGATTAATAGGCCGTGG
GCGTGACTCGAACCTCATGCACAAAAACGAGGATGTGCCTGAGATAGAAGGTCACC
AGACGACAGAGCTCACCATAGTAGTGCACAGCTTTATGGTCCTACACCAAGTCCACC
TCCTCAACAACGCGGGGCTATAGGTGTTTGCAAATTTCTTCCATGCGTGGCTTGCAA
TAGTGCTTGCATGCTGCAACAATGCATATGGGGTAATCAAAACGAGCACTCGCGAG
CATTCCATCAAAGGTGAGAAATATTATTCTAAGCTTTTTCTGGAAGCAAACATAGGC
CGCGATCACGCACCAAAATTGAGCCGGATTAAGAATGGGCTTCCCCCACTTCATATT
TGTGTGGCATTAGGACTATATATAGTTGATACATTCTCGACACACTAGTGTCCATCA
TC
SEQ ID NO: 166-pMAL11
GCGCCTCAAGAAAATGATGCTGCAAGAAGAATTGAGGAAGGAACTATTCATCTTAC
GTTGTTTGTATCATCCCACGATCCAAATCATGTTACCTACGTTAGGTACGCTAGGAA
CTAAAAAAAGAAAAGAAAAGTATGCGTTATCACTCTTCGAGCCAATTCTTAATTGTG
TGGGGTCCGCGAAAATTTCCGGATAAATCCTGTAAACTTTAACTTAAACCCCGTGTT
TAGCGAAATTTTCAACGAAGCGCGCAATAAGGAGAAATATTATCTAAAAGCGAGAG
TTTAAGCGAGTTGCAAGAATCTCTACGGTACAGATGCAACTTACTATAGCCAAGGTC
TATTCGTATTACTATGGCAGCGAAAGGAGCTTTAAGGTTTTAATTACCCCATAGCCA
TAGATTCTACTCGGTCTATCTATCATGTAACACTCCGTTGATGCGTACTAGAAAATG
ACAACGTACCGGGCTTGAGGGACATACAGAGACAATTACAGTAATCAAGAGTGTAC
CCAACTTTAACGAACTCAGTAAAAAATAAGGAATGTCGACATCTTAATTTTTTATAT
AAAGCGGTTTGGTATTGATTGTTTGAAGAATTTTCGGGTTGGTGTTTCTTTCTGATGC
TACATAGAAGAACATCAAACAACTAAAAAAATAGTATAAT
SEQ ID NO: 167-pMAL12
ATTATACTATTTTTTTAGTTGTTTGATGTTCTTCTATGTAGCATCAGAAAGAAACACC
AACCCGAAAATTCTTCAAACAATCAATACCAAACCGCTTTATATAAAAAATTAAGAT
GTCGACATTCCTTATTTTTTACTGAGTTCGTTAAAGTTGGGTACACTCTTGATTACTG
TAATTGTCTCTGTATGTCCCTCAAGCCCGGTACGTTGTCATTTTCTAGTACGCATCAA
CGGAGTGTTACATGATAGATAGACCGAGTAGAATCTATGGCTATGGGGTAATTAAA
ACCTTAAAGCTCCTTTCGCTGCCATAGTAATACGAATAGACCTTGGCTATAGTAAGT
TGCATCTGTACCGTAGAGATTCTTGCAACTCGCTTAAACTCTCGCTTTTAGATAATAT
TTCTCCTTATTGCGCGCTTCGTTGAAAATTTCGCTAAACACGGGGTTTAAGTTAAAGT
TTACAGGATTTATCCGGAAATTTTCGCGGACCCCACACAATTAAGAATTGGCTCGAA
GAGTGATAACGCATACTTTTCTTTTCTTTTTTTAGTTCCTAGCGTACCTAACGTAGGT
AACATGATTTGGATCGTGGGATGATACAAACAACGTAAGATGAATAGTTCCTTCCTC
AATTCTTCTTGCAGCATCATTTTCTTGAGGCGCTCTGGGCAAGGTATAAAAAGTTCC
ATTAATACGTCTCTAAAAAATTAAATCATCCATCTCTTAAGCAGTTTTTTTGATAATC
TCAAATGTACATCAGTCAAGCGTAACTAAATTACATAA
SEQ ID NO: 168-pMAL31
TTATGTATTTTAGTTACGCTTGACTGATGTACATTTGAGATTATCAAAAAAACTGCTT
AAGAGATAGATGGTTTAATTTTTTAGAGACGTATTAATGGAACTTTTTATACCTTGCC
CAGAGCGCCTCAAGAAAATGATGCTGAAAGAAGAATTGAGGAAGGAACTACTCATC
TTACGTTGTTTGTATCATCCCACGATCCAAATCATGTTACCTACGTTAGGTACGCTAG
GAACTGAAAAAAGAAAAGAAAAGTATGCGTTATCACTCTTCGAGCCAATTCTTAATT
GTGTGGGGTCCGCGAAAACTTCCGGATAAATCCTGTAAACTTAAACTTAAACCCCGT
GTTTAGCGAAATTTTCAACGAAGCGCGCAATAAGGAGAAATATTATATAAAAGCGA
GAGTTTAAGCGAGGTTGCAAGAATCTCTACGGTACAGATGCAACTTACTATAGCCAA
GGTCTATTCGTATTGGTATCCAAGCAGTGAAGCTACTCAGGGGAAAACATATTTTCA
GAGATCAAAGTTATGTCAGTCTCTTTTTCATGTGTAACTTAACGTTTGTGCAGGTATC
ATACCGGCCTCCACATAATTTTTGTGGGGAAGACGTTGTTGTAGCAGTCTCCTTATA
CTCTCCAACAGGTGTTTAAAGACTTCTTCAGGCCTCATAGTCTACATCTGGAGACAA
CATTAGATAGAAGTTTCCACAGAGGCAGCTTTCAATATACTTTCGGCTGTGTACATT
TCATCCTGAGTGAGCGCATATTGCATAAGTACTCAGTATATAAAGAGACACAATATA
CTCCATACTTGTTGTGAGTGGTTTTAGCGTATTCAGTATAACAATAAGAATTACATCC
AAGACTATTAATTAACT
SEQ ID NO: 169-pMAL32
AGTTAATTAATAGTCTTGGATGTAATTCTTATTGTTATACTGAATACGCTAAAACCAC
TCACAACAAGTATGGAGTATATTGTGTCTCTTTATATACTGAGTACTTATGCAATATG
CGCTCACTCAGGATGAAATGTACACAGCCGAAAGTATATTGAAAGCTGCCTCTGTGG
AAACTTCTATCTAATGTTGTCTCCAGATGTAGACTATGAGGCCTGAAGAAGTCTTTA
AACACCTGTTGGAGAGTATAAGGAGACTGCTACAACAACGTCTTCCCCACAAAAAT
TATGTGGAGGCCGGTATGATACCTGCACAAACGTTAAGTTACACATGAAAAAGAGA
CTGACATAACTTTGATCTCTGAAAATATGTTTTCCCCTGAGTAGCTTCACTGCTTGGA
TACCAATACGAATAGACCTTGGCTATAGTAAGTTGCATCTGTACCGTAGAGATTCTT
GCAACCTCGCTTAAACTCTCGCTTTTATATAATATTTCTCCTTATTGCGCGCTTCGTT
GAAAATTTCGCTAAACACGGGGTTTAAGTTTAAGTTTACAGGATTTATCCGGAAGTT
TTCGCGGACCCCACACAATTAAGAATTGGCTCGAAGAGTGATAACGCATACTTTTCT
TTTCTTTTTTCAGTTCCTAGCGTACCTAACGTAGGTAACATGATTTGGATCGTGGGAT
GATACAAACAACGTAAGATGAGTAGTTCCTTCCTCAATTCTTCTTTCAGCATCATTTT
CTTGAGGCGCTCTGGGCAAGGTATAAAAAGTTCCATTAATACGTCTCTAAAAAATTA
AACCATCTATCTCTTAAGCAGTTTTTTTGATAATCTCAAATGTACATCAGTCAAGCGT
AACTAAAATACATAA
SEQ ID NO: 170-CBGaS from Stachybotrys chartarum
MSAKVSPMAYTNPRYETGPLSLIPKPIVPYFELMRFELPHGYYLGYFPHLVGIMYGASA
GPERLPARDLVFQALLYVGWTFAMRGAGCAWNDNIDQDFDRKTERCRTRPIARGAVST
TAGHVFAVAGVALAFLCLSPLPTECHQLGVLFTVLSVIYPFCKRFTNFAQVILGMTLAA
NFILAAYGAGLPALEQPYTRPTMSATLAITLLVVFYDVVYARQDTADDLKSGVKGMAV
LFRNHIEVLLAVLTCTIGGLLAATGVSVGNGPYYFLFSVAGLTVALLAMIGGIRYRIFHT
WNGYSGWFYVLAIINLMSGYFIEYLDNAPILARGS
SEQ ID NO: 171-Geranyl pyrophosphate synthase from Streptomyces aculeolatus
MTTEVTSFTGAGPHPAASVRRITDDLLQRVEDKLASFLTAERDRYAAMDERALAAVDA
LTDLVTSGGKRVRPTFCITGYLAAGGDAGDPGIVAAAAGLEMLHVSALIHDDILDNSAQ
RRGKPTIHTLYGDLHDSHGWRGESRRFGEGIGILIGNLALVYSQELVCQAPPAVLAEWH
RLCSEVNIGQCLDVCAAAEFSADPELSRLVALIKSGRYTIHRPLVMGANAASRPDLAAA
YVEYGEAVGEAFQLRDDLLDAFGDSTETGKPTGLDFTQHKMTLLLGWAMQRDTHIRTL
MTEPGHTPEEVRRRLEDTEVPKDVERHIADLVEQGRAAIADAPIDPQWRQELADMAVR
AAYRTN
SEQ ID NO: 172-Linker
EPEPEPEPEPEPEPEASAKALLSQPLLLI

1. A genetically modified host cell capable of producing CBDa or CBD, wherein the genetically modified host cell comprises one or more heterologous nucleic acids that each, independently, encodes an enzyme having CBDaS activity.

2. The genetically modified host cell of claim 1, wherein the enzyme having CBDaS activity is a fusion protein.

3. The genetically modified host cell of claim 2, wherein the fusion protein comprises an amino acid sequence of a CBDaS or a portion thereof.

4. The genetically modified host cell of claim 2, wherein the fusion protein comprises an amino acid sequence that is at least 80% identical to the amino acid sequence of SEQ ID NOS: 1, 4, 7, 8, 9, 10, 11, 13, 15, 17, 19, 21, 4, 7, 8, 9, 10, 11, 13, 15, 17, 19, 21, 134, 135, 136, 137, 147, 148, 149, 150, or 151.

5. The genetically modified host cell of claim 2, wherein the fusion protein comprises an amino acid sequence of a carrier protein or a portion thereof, an amino acid sequence of a signal sequence or a portion thereof, or an amino acid sequence of a linker or a portion thereof.

6. The genetically modified host cell of claim 2, wherein the fusion protein comprises an amino acid sequence that is at least 80% identical to the amino acid sequence of SEQ ID NOS: 34, 36, 73, 77, 81, 87, 89, 90, 91, 103, 104, 105, 106, 107, 108, 109, 110, 111, or 112.

7. (canceled)

8. The genetically modified host cell of claim 2, wherein the fusion protein comprises an amino acid sequence that is at least 80% identical to the amino acid sequence of SEQ ID NOS: 42, 43, 44, 45, 46, 47, 48, 49, 50, 51, 52, 53, or 54.

9. (canceled)

10. The genetically modified host cell of claim 2, wherein the fusion protein comprises an amino acid sequence that is at least 80% identical to the amino acid sequence of SEQ ID NOS: 113, 114, 115, 116, 117, 118, 119, 120, 121, 122, 123, 124, 125, 152, or 172.

11. The genetically modified host cell of claim 2, wherein the fusion protein comprises an amino acid sequence of a protease recognition site.

12. The genetically modified host cell of claim 11, wherein the protease recognition site is selected from the group of amino acid sequences consisting of RR, KR, RRK, RRQ, RRW, RRE, LDKR, LDKREAEA, and KREAEA.

13. The genetically modified host cell of claim 2, wherein the fusion protein comprises an amino acid sequence of a mating factor alpha (MFα) or a portion thereof.

14. The genetically modified host cell of claim 2, wherein the fusion protein comprises an amino acid sequence that is at least 80% identical to the amino acid sequence of SEQ ID NOS: 153, 154, 155, 156, or 157.

15. The genetically modified host cell of claim 2, wherein the fusion protein comprises two or more of:
a) an amino acid sequence of a CBDaS or a portion thereof;
b) an amino acid sequence that is at least 80% identical to the amino acid sequence of SEQ ID NOS: 1, 4, 7, 8, 9, 10, 11, 13, 15, 17, 19, 21, 4, 7, 8, 9, 10, 11, 13, 15, 17, 19, 21, 134, 135, 136, 137, 147, 148, 149, 150, or 151;
c) an amino acid sequence of a carrier protein or a portion thereof;
d) an amino acid sequence that is at least 80% identical to the amino acid sequence of SEQ ID NOS: 34, 36, 73, 77, 81, 87, 89, 90, 91, 103, 104, 105, 106, 107, 108, 109, 110, 111, or 112;
e) an amino acid sequence of a signal sequence or a portion thereof;
f) an amino acid sequence that is at least 80% identical to the amino acid sequence of SEQ ID NOS: 42, 43, 44, 45, 46, 47, 48, 49, 50, 51, 52, 53, or 54;
g) an amino acid sequence of a linker or a portion thereof;
h) an amino acid sequence at least 80% identical to the amino acid sequence of SEQ ID NOS: 113, 114, 115, 116, 117, 118, 119, 120, 121, 122, 123, 124, 125, 152, or 172;
i) an amino acid sequence of a protease recognition site;
j) a protease recognition site selected from the group of amino acid sequences consisting of RR, KR, RRK, RRQ, RRW, RRE, LDKR, LDKREAEA, and KREAEA;
k) an amino acid sequence of a mating factor alpha (MF) or a portion thereof; or
l) an amino acid sequence that is at least 80% identical to the amino acid sequence of SEQ ID NOS: 153, 154, 155, 156, or 157.

16. The genetically modified host cell of claim 1, wherein the enzyme having CBDaS activity or the amino acid sequence of a CBDaS or a portion thereof comprises one or more of the following mutations when aligned with and in reference to SEQ ID NO: 136: N45Q, N65Q, S168N, N296Q, N304Q, N328Q, N498Q, T47S, T67S, S170T, T298S, T306S, S330T, or T500S.

17. The genetically modified host cell of claim 1, wherein the enzyme having CBDaS activity or the amino acid sequence of a CBDaS or a portion thereof comprises one or more amino acid substitutions occurring at position(s) 29, 31, 43, 49, 53, 56, 57, 65, 71, 78, 79, 93, 95, 103, 117, 125, 129, 143, 147, 151, 161, 183, 213, 235, 241, 263, 264, 285, 286, 303, 314, 325, 336, 339, 396, 436, 518, or 540 when aligned with and in reference to SEQ ID NO: 137.

18. (canceled)

19. The genetically modified host cell of claim 1, wherein the enzyme having CBDaS activity or the amino acid sequence of a CBDaS or a portion thereof comprises one or more amino acid substitutions selected from the group consisting of:
a) R53T, N78D, V147D, H235D, I263V, K325N, V540C;
b) R53T, P65D, L71D, N79D, L93D, V147D, W183N, H235D, S336C;
c) L71D, N78D, G117A, V147D, W183N, I263V, K325N, S336C, V540C;
d) R53T, P65D, L71D, N78D, N79D, L93D, V147D, W183N, H235D, K325N, S336C, V540C;
e) L71D, L93D, V147D, H235D, I263V;
f) R53T, V147D, 1151L, W183N, H235D, S336C, V540C;
g) R53T, N78D, N79D, G117A, V147D, S336C;
h) R53T, P65D, L71D, N79D, L93D, V147D, W183N, H235D, S336C;
i) R53T, L71D, N78D, G117A, V147D, H235D, S336C, V540C;
j) R53T, P65D, N78D, G117A, V147D, H235D, K325N, S336C, V540C;
k) R53T, P65D, N78D, L93D, V147D, W183N, H235D, V540C;
1, R53T, N78D, V147D, W183N, H235D, I263V, S336C;
m) R53T, N79D, V147D, W183N, H235D, I263V, K325N, S336C;
n) R53T, P65D, L71D, N78D, V147D, H235D, I263V, S336C, V540C;
0, R53T, L71D, G117A, V147D, H235D, I263V, V540C;
p) R53T, L71D, N78D, G117A, V147D, H235D, 1263V, K325N, S336C, V540C;
q) R53T, P65D, N78D, N79D, V147D, S336C, V540C;
r) R53T, N78D, N79D, V147D, W183N, H235D, I263V, K325N;
s) R53T, 1151L, H235D, K325N, S336C; and
t) R53T, P65D, L71D, N79D, L93D, V147D, W183N, H235D, S336C,
when aligned with and in reference to SEQ ID NO: 137.

20. A genetically modified host cell comprising an enzyme having at least 80% sequence identity to the amino acid sequence of any of the enzymes having CBDaS activity or to the amino acid sequence of a CBDaS or a portion thereof in claim 19.

21-22. (canceled)

23. A method for producing CBDa or CBD, comprising:
a) culturing the genetically modified host cell of claim 1 in a medium with a carbon source under conditions suitable for making CBDa or CBD; and
b) recovering CBDa or CBD from the genetically modified host cell or the medium.

24. A fermentation composition comprising CBDa or CBD, comprising:
a) the genetically modified host cell of claim 1; and
b) CBDa or CBD produced by the genetically modified host cell.

25. (canceled)

26. A non-naturally occurring enzyme having CBDaS activity, comprising an amino acid sequence that is at least 80% identical to the amino acid sequence of SEQ ID NO: 4, 7, 8, 9, 10, 11, 13, 15, 17, 19, 21, 134, 135, 136, 137, 147, 148, 149, 150, or 151.

27-51. (canceled)
